SEQUENCE LISTING (41 sequences)

SEQ ID NO: 1 - protein, 306 aa, Homo sapiens
IWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCS

SEQ ID NO: 2 - DNA, 1397 bp, Homo sapiens
  CDS (41)...(1024)
gtttcagggccattggactctccgtcctgcccagagcaagatgtgtcaccagcagttggtcatctcttggttttccctggtttttctggcatctcccctcgtggccatatgggaactgaagaaagatgtttatgtcgtagaattggattggtatccggatgcccctggagaaatggtggtcctcacctgtgacacccctgaagaagatggtatcacctggaccttggaccagagcagtgaggtcttaggctctggcaaaaccctgaccatccaagtcaaagagtttggagatgctggccagtacacctgtcacaaaggaggcgaggttctaagccattcgctcctgctgcttcacaaaaaggaagatggaatttggtccactgatattttaaaggaccagaaagaacccaaaaataagacctttctaagatgcgaggccaagaattattctggacgtttcacctgctggtggctgacgacaatcagtactgatttgacattcagtgtcaaaagcagcagaggctcttctgacccccaaggggtgacgtgcggagctgctacactctctgcagagagagtcagaggggacaaacaaggatatgagtactcagtggagtgccaggaggacagtgcctgcccagctgctgaggagagtctgcccattgaggtcatggtggatgccgttcacaagctcaagtatgaaaactacaccagcagcttcttcatcagggacatcatcaaacctgacccacccaagaacttgcagctgaagccattaaagaattctcggcaggtggaggtcagctgggagtaccctgacacctggagtactccacattcctacttctccctgacattctgcgttcaggtccagggcaagagcaagagagaaaagaaagatagagtcttcacggacaagacctcagccacggtcatctgccgcaaaaatgccagcattagcgtgcgggcccaggaccgctactatagctcatcttggagcgaatgggcatctgtgccctgcagttaggttctgatccaggatgaaaatttggaggaaaagtggaagatattaagcaaaatgtttaaagacacaacggaatagacccaaaaagataatttctatctgatttgctttaaaacgtttttttaggatcacaatgatatctttgctgtatttgtatagttagatgctaaatgctcattgaaacaatcagctaatttatgtatagattttccagctctcaagttgccatgggccttcatgctatttaaatatttaagtaatttatgtatttattagtatattactgttatttaacgtttgtctgccaggatgtatggaatgtttcatactcttatgacctgatccatcaggatcagtccctattatgcaaaat

SEQ ID NO: 3 - protein, 328 aa, Homo sapiens
MCHQQLVISWFSLVFLASPLVAIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDKQGYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCS

SEQ ID NO: 4 - DNA, 856 bp, Homo sapiens
  CDS (170)...(826)
gaattcccagaaagcaagagaccagagtcccgggaaagtcctgccgcgcctcgggacaattataaaaatgtggccccctgggtcagcctcccagccaccgccctcacctgccgcggccacaggtctgcatccagcggctcgccctgtgtccctgcagtgccggctcagcatgtgtccagcgcgcagcctcctccttgtggctaccctggtcctcctggaccacctcagtttggccagaaacctccccgtggccactccagacccaggaatgttcccatgccttcaccactcccaaaacctgctgagggccgtcagcaacatgctccagaaggccagacaaactctagaattttacccttgcacttctgaagagattgatcatgaagatatcacaaaagataaaaccagcacagtggaggcctgtttaccattggaattaaccaagaatgagagttgcctaaattccagagagacctctttcataactaatgggagttgcctggcctccagaaagacctcttttatgatggccctgtgccttagtagtatttatgaagacttgaagatgtaccaggtggagttcaagaccatgaatgcaaagcttctgatggatcctaagaggcagatctttctagatcaaaacatgctggcagttattgatgagctgatgcaggccctgaatttcaacagtgagactgtgccacaaaaatcctcccttgaagaaccggatttttataaaactaaaatcaagctctgcatacttcttcatgctttcagaattcgggcagtgactattgacagagtgacgagctatctgaatgcttcctaaaaagcgaggtccctccaaaccgttgtc

SEQ ID NO: 5 - protein, 219 aa, Homo sapiens
MCPARSLLLVATLVLLDHLSLARNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVTSYLNAS

SEQ ID NO: 6 - protein, 20 aa, Homo sapiens
  SITE (1)...(2): Xaa=undetermined residue
XXLPVATPDPGMFPXLHHSQ

SEQ ID NO: 7 - protein, 23 aa, Homo sapiens
IWELKKDVYVVELDWYPDAPGEM

SEQ ID NO: 8 - protein, 6 aa, Homo sapiens
NKTFLR

SEQ ID NO: 9 - protein, 19 aa, Homo sapiens
  SITE (10)...(10): Xaa=undetermined residue
GSSDPQGVTXGAATLSAER

SEQ ID NO: 10 - protein, 13 aa, Homo sapiens
  SITE (12)...(12): Xaa=undetermined residue
VFTDKTSATVIXR

SEQ ID NO: 11 - protein, 7 aa, Homo sapiens
TLTIQVK

SEQ ID NO: 12 - protein, 11 aa, Homo sapiens
NLQLKPLKNSR

SEQ ID NO: 13 - protein, 6 aa, Homo sapiens
IWELKK

SEQ ID NO: 14 - protein, 8 aa, Homo sapiens
AQDRYYSS

SEQ ID NO: 15 - protein, 17 aa, Homo sapiens
KEDGIWSTDILKDQKEP

SEQ ID NO: 16 - protein, 13 aa, Homo sapiens
  SITE (5)...(5): Xaa=undetermined residue
LKYEXYTSSFFIR

SEQ ID NO: 17 - protein, 12 aa, Homo sapiens
  SITE (6)...(6): Xaa=undetermined residue
KEDGIXSTDILK

SEQ ID NO: 18 - protein, 18 aa, Homo sapiens
  SITE (12)...(12): Xaa=undetermined residue
AQDRYYSSSWEXASVPXX

SEQ ID NO: 19 - protein, 15 aa, Homo sapiens
  SITE (1)...(1): Xaa=Gly residue was determined to be the most likely or “best-guess” at this position
GGEVLSHSLLLLHKK

SEQ ID NO: 20 - protein, 9 aa, Homo sapiens
LKKDVYVVE

SEQ ID NO: 21 - protein, 10 aa, Homo sapiens
LDWYPDAPGE

SEQ ID NO: 22 - protein, 14 aa, Homo sapiens
  SITE (14)...(14): Xaa=Glu residue was determined to be the most likely or “best-guess” at this position
VLGSGKTLTIQVKE

SEQ ID NO: 23 - protein, 26 aa, Homo sapiens
  SITE (1)...(1): Xaa=undetermined residue
XNLPVATPDPGMFPXLHHSQNLLRAV

SEQ ID NO: 24 - protein, 9 aa, Homo sapiens
DIIKPDPPK

SEQ ID NO: 25 - protein, 24 aa, Homo sapiens
  SITE (11)...(11): Xaa=undetermined residue
VDAVHKLKYEXYTSSFFIRDIIKP

SEQ ID NO: 26 - DNA, 23 bp, Artificial Sequence
  Description of Artificial Sequence forward primer
ctcgaattcgarytnaaraarga

SEQ ID NO: 27 - DNA, 24 bp, Artificial Sequence
  Description of Artificial Sequence reverse primer
ctcgaattcnggngcrtcnggrta

SEQ ID NO: 28 - DNA, 36 bp, Artificial Sequence
  Description of Artificial Sequence oligonucleotide
gagctaaagaaagatgtttatgtcgtagaattggat

SEQ ID NO: 29 - DNA, 36 bp, Artificial Sequence
  Description of Artificial Sequence oligonucleotide
aggggcatccggataccaatccaattctacgacata

SEQ ID NO: 30 - DNA, 23 bp, Artificial Sequence
  Description of Artificial Sequence forward primer
ctcgaattcgayccnggnatgtt

SEQ ID NO: 31 - DNA, 24 bp, Artificial Sequence
  Description of Artificial Sequence reverse primer
ctcgaattcngcncknarnarrtt

SEQ ID NO: 32 - DNA, 34 bp, Artificial Sequence
  Description of Artificial Sequence oligonucleotide
gatccgggaatgttcccatgccttcaccactccc

SEQ ID NO: 33 - DNA, 47 bp, Artificial
  Description of Artificial Sequence Primer
ttaggcttaggcttaggcttaggcttaggcttaggcttaggcttagg

SEQ ID NO: 34 - DNA, 21 bp, Artificial
  Description of Artificial Sequence Primer
gaataatgagaattttcaggc

SEQ ID NO: 35 - DNA, 22 bp, Artificial
  Description of Artificial Sequence Primer
attccttctgtgtactgttgcc

SEQ ID NO: 36 - DNA, 22 bp, Artificial
  Description of Artificial Sequence Primer
gatattgacgacctagatgacg

SEQ ID NO: 37 - DNA, 22 bp, Artificial
  Description of Artificial Sequence Primer
tctgattttgacgatatttcgc

SEQ ID NO: 38 - DNA, 20 bp, Artificial
  Description of Artificial Sequence Primer
aactgacggacttgtgtccc

SEQ ID NO: 39 - DNA, 30 bp, Artificial Sequence
  Description of Artificial Sequence oligonucleotide
ctgaagccattaaagaattctcggcaggtg

SEQ ID NO: 40 - protein, 20 aa, Homo sapiens
  Description of Artificial Sequence Positions 1 - 20 of purified 40 kDa protein SEQ ID NO 1
IWELKKDVYVVELDWYPDAP

SEQ ID NO: 41 - DNA, 18 bp, Artificial
  Description of Artificial Sequence Primer
ttagggttagggttaggg

(19) United States
(12) Patent Application Publication
(10) Pub. No.: US 2003/0199002 A1
(43) Pub. Date: Oct. 23, 2003

(54) Clk-2 nucleic acids, polypeptides and uses thereof

(76) Inventors: Siegfried Hekimi, Montreal (CA); Claire Benard, Montreal (CA); Ning Jiang, Montreal (CA); Hania Kebir, Montreal (CA); Brenton McCright, Gaithersburg, MD (US); Bernard Lakowski, Paris (FR)

Correspondence Address: PENNIE AND EDMONDS, 1155 AVENUE OF THE AMERICAS, NEW YORK, NY, 100362711

(21) Appl. No.: 10/349,507

(22) Filed: Jan. 22, 2003

Related U.S. Application Data
(63) Continuation-in-part of application No. 10/312,187, filed on Apr. 9, 2003.
Application No. 10/312,187 is a 371 of international application No. PCT/CA01/00913, filed on Jun. 20, 2001.
(60) Provisional application No. 60/213,174, filed on Jun. 22, 2000.
(60) Provisional application No. 60/254,932, filed on Dec. 13, 2000.

(51) Int. Cl. G01N 33/53; G01N 33/567; C07H 21/04; C12P 21/04; C12N 9/10; C12N 5/06

(52) U.S. Cl. 435/7.2; 435/69.7; 435/193; 435/320.1; 435/325; 536/23.2

(57) ABSTRACT

The present invention relates to nucleotide sequences of clk-2 genes, particularly human clk-2, and amino acid sequences of their encoded proteins, as well as derivatives and analogs thereof. The present invention also relates to methods and compositions designed for the treatment, management, or prevention of disorders associated with abnormal expression and/or activity of clk-2 nucleic acids and/or proteins. In one embodiment, the invention encompasses a method of treating or preventing a disorder associated with decreased apoptosis (e.g., cancer, autoimmune disorders) or decreased telomere length (e.g., rapid aging or advanced age) by administering to a subject in need thereof an effective amount of an agent that promotes clk-2 activity. In another embodiment, invention encompasses a method of treating or preventing a disorder associated with increased apoptosis (e.g., neurodegenerative disorders) and increased telomere length (e.g., cancer) such as by administering to a subject in need thereof an effective amount of an agent that decreases clk-2 function. Diagnostic methods and methods for screening for therapeutically useful agents are also provided.

This application is a continuation-in-part application of U.S. patent application Ser. No. 10/312,187 filed Dec. 20, 2002 which claims the benefit of priority to International Patent Application No. PCT/CA01/00913 filed Jun. 20, 2001, which claims the benefit of priority to U.S. Provisional Patent Application Serial No. 60/213,174 filed Jun. 22, 2000 and No. 60/254,932 filed Dec. 13, 2000, each of which is incorporated herein by reference in its entirety.

### 1. FIELD OF THE INVENTION

The present invention relates to nucleotide sequences of clk-2 genes, particularly human clk-2, and amino acid sequences of their encoded proteins, as well as derivatives (e.g., fragments) and analogs thereof. The present invention also relates to methods and compositions designed for the treatment, management, or prevention of disorders associated with abnormal expression and/or activity of clk-2 nucleic acids and/or proteins. Diagnostic methods and methods for screening for therapeutically useful agents are also provided.

### 2. BACKGROUND OF THE INVENTION

A class of genes was identified in the nematode Caenorhabditis elegans, the clk (‘clock’) genes, whose activity controls how fast the worms live and die. Mutations in these genes result in an alteration of developmental and behavioral timing, including an average slow down of the animals' embryonic and post-embryonic development and of their rhythmic behaviors, as well as an increase in the animal's life span. In addition, mutations in these genes display a maternal effect, namely, homozygous mutants (clk/clk) derived from a heterozygous mother (clk/+), appear phenotypically wild-type.

Mutations that define the genes clk-1, clk-2, clk-3 were isolated in a screen for viable maternal-effect mutations in the nematode Caenorhabditis elegans (Hekimi et al., 1995, Genetics 141:1351). gro-1 was originally identified by a spontaneous mutation isolated from a strain that had been recently established from a wild isolate (Hodgkin & Doniach, 1997, Genetics 146:149). Subsequent reappraisal of this mutation revealed that it shares the characteristics of the elk genes (Wong et al., 1995, Genetics 139:1247).

Two of these genes, clk-1 and gro-1, have been molecularly identified. clk-1 encodes a protein that is highly conserved from proteobacteria to humans which is structurally similar to the yeast protein Coq7p (Ewbank et al, 1997, Science 275:980; International Patent Publication No. WO98/17823). gro-1 encodes a highly conserved cellular enzyme, dimethylallyltransferase:tRNA dimethylallyltransferase (International Patent Publication No. WO99/10482).

To date, clk-1 is the gene that has been characterized in greatest detail. In addition to the phenotypic and molecular characterization, it was found that clk-1 is ubiquitously expressed in the worm's body where it localizes to the mitochondria (Felkai et al, 1999, EMBO J. 18:1783). clk-1 thus controls timing by regulating physiological rates (Branicky et al., 2000, Bioessays 22:48).

The gene clk-2 is defined by one allele that was isolated in a screen for viable maternal-effect mutations in Caenorhabditis elegans (Hekimi et al., 1995, Genetics 141:1351). The mutations in the gene clk-2 were shown to result in an alteration of the timing of several developmental and behavioral events (Hekimi et al., 1995, Genetics 141: 1351) and that the activity of the gene clk-2 controls how fast the worms live and how soon they die (Lakowski & Hekimi, 1996, Science 272:1010). Additional phenotypes of the clk-2 mutants have been observed, such as the temperature sensitivity of the clk-2(qm37) allele. Overall, these phenotypes are similar to those of mutations in the three elk genes (Hekimi et al., 1995, Genelics 141:1351).

It would be highly desirable to be provided with a detailed phenotypic and molecular characterization of the clk-2 gene in a mammalian system.

### 3. SUMMARY OF THE INVENTION

The present invention relates to nucleotide sequences of clk-2 genes, particularly human clk-2, and amino acid sequences of their encoded proteins, as well fragments, derivatives and analogs which are functionally active, i.e., they are capable of displaying one or more known functional activities associated with a full-length wild-type clk-2 protein. Such functional activities include but are not limited to antigenicity, immunogenicity, and biological (e.g., modulation of growth, telomere length, and apoptosis). In one embodiment, the invention encompasses an isolated clk-2 nucleic acid molecule that comprises a nucleotide sequence which is at least 90% identical to the nucleotide sequence of any of SEQ ID NOs: 1, 4, 5, 6, 7, 15, 16, 20, 21, 22, 23, 24; that hybridizes with a nucleic acid probe consisting of the nucleotide sequence of any of SEQ ID NOs:1, 4, 5, 6, 7, 15, 16, 20, 21, 22, 23, 24, or a complement thereof under stringent conditions; or that comprises a nucleic acid molecule that encodes a polypeptide comprising the amino acid sequence of any of SEQ ID NOs:2, 3, 8, 9, 10, 11, 12, 13, 14, 17, 18, 19, 25, 26, 27, 28, 29, 30, 31, 32. Complements, fragments and variants of the isolated clk-2 nucleic acid molecule are also encompassed.

In another embodiment, the invention encompasses an isolated clk-2 polypeptide comprising a portion of the amino acid sequence of any of SEQ ID NOs:2, 3, 8, 9, 10, 11, 12, 13, 14, 17, 18, 19, 25, 26, 27, 28, 29, 30, 31, 32; a naturally occurring allelic variant of and a variant that is at least 90% identical to a clk-2 polypeptide comprising the amino acid sequence of any of SEQ ID NOs:2, 3, 8, 9, 10, 11, 12, 13, 14, 17, 18, 19, 25, 26, 27, 28, 29, 30, 31, 32.

Methods of production of the clk-2 proteins, derivatives and analogs, e.g., by recombinant means, are also provided.

The invention also provides methods for the identification of agents which bind to and/or modulate the expression of clk-2 gene or the activity (e.g., modulation of growth, telomere length, and apoptosis) or phosphorylation level of clk-2 protein in cells that are involved in clk-2 related disorders and processes relevant to cancer, neurodegenerative disorders, autoimmune disorders, and aging.

In one embodiment, the invention encompasses a method for identifying a compound that specifically binds with a clk-2 polypeptide comprising contacting the polypeptide with a compound under conditions and for a sufficient period of time that allows binding between the polypeptide and the compound; and detecting binding of the compound to the polypeptide.

In another embodiment, the invention encompasses a method for identifying a compound which modulates the activity of a clk-2 polypeptide comprising contacting a cell expressing a clk-2 polypeptide with a compound under conditions and for a sufficient period of time for the compound to enter the cell; and determining the activity of the clk-2 polypeptide in the cell; wherein a difference in the activity of the clk-2 polypeptide as compared to the activity of the clk-2 polypeptide in the absence of the compound indicates that the compound modulates the activity of the clk-2 polypeptide.

In yet another embodiment, the invention encompasses a method of identifying a compound that modulates clk-2 expression comprising contacting a recombinant cell with a compound, said recombinant cell comprising a reporter gene operably associated with a regulatory sequence of a clk-2 gene, such that expression of the reporter gene is regulated by the regulatory sequence; and determining the level of expression of said reporter gene in said contacted recombinant cell, wherein a difference in the expression level of said reporter gene in said contacted recombinant cell as compared to the expression level of said reporter gene in said recombinant cell not contacted with the compound indicates that the compound modulates clk-2 expression

In yet another embodiment, the invention encompasses a method of identifying a compound that modulates the expression of a clk-2 nucleic acid or polypeptide comprising contacting a cell with a compound, and determining the level of expression of the clk-2 nucleic acid or polypeptide in said contacted recombinant cell, wherein a difference in the expression level of the clk-2 nucleic acid or polypeptide in said contacted recombinant cell as compared to the expression level of the clk-2 nucleic acid or polypeptide in said recombinant cell not contacted with the compound indicates that the compound modulates clk-2 expression.

In another embodiment, the invention encompasses a method for identifying a compound which modulates the activity of a clk-2 polypeptide comprising contacting a cell or organism with a compound, wherein the cell or organism exhibits at least one phenotype that is altered as a result of its expression of a mutant clk-2 polypeptide, when compared to a wild type cell or organism; and determining the phenotype of said contacted cell or organism.

The invention also relates to therapeutic and diagnostic methods and compositions based on clk-2 proteins and nucleic acids. Therapeutic agents of the invention include but are not limited to clk-2 proteins and analogs and derivatives (including fragments) thereof; antibodies thereto; nucleic acids encoding the clk-2 proteins, analogs, or derivatives; and clk-2 antisense nucleic acids. Methods for targeting these therapeutic agents to specific organs, tissues, cell types, subcellular locations, as well as tumors, metastatic lesions, degenerating neurons, autoimmune lymphocytes, and aged cells, are provided. Animal models, diagnostic methods and screening methods for predisposition to disorders, are also provided by the invention. For example, the methods and compositions described herein can also be used to regulate apoptosis in various types of tissue, such as tumor tissue including, but not limited to tumors that exhibit abnormal telomere length or cell cycle characteristics.

The invention thus provides for treatment of clk-2-related disorders that are associated with decreased apoptosis (e.g., cancer, autoimmune disorders) or decreased telomere length (e.g., rapid aging or advanced age) by administering to a subject in need thereof an effective amount of an agent that promotes clk-2 activity (e.g., clk-2, an agonist of clk-2; nucleic acids that encode clk-2). In one embodiment, the invention encompasses a method of treating or preventing a disorder associated with excess clk-2 polypeptide activity in a subject comprising administering to a subject in which such treatment or prevention is desired an effective amount of a compound that decreases clk-2 polypeptide activity or clk-2 gene expression.

In another embodiment, the invention encompasses a method of treating or preventing a disorder associated with deficient clk-2 polypeptide activity in a subject comprising administering to a subject in which such treatment or prevention is desired an effective amount of a compound that increases clk-2 polypeptide activity or clk-2 gene expression.

The invention also provides methods of treatment of clk-2-related disorders that are associated with increased apoptosis (e.g., neurodegenerative disorders) and increased telomere length (e.g., cancer) such as by administering to a subject in need thereof an effective amount of an agent that antagonizes, inhibits clk-2 function (e.g., antibodies, antisense nucleic acids). The methods and compositions described herein can also be used to affect the rate and effect of aging and other disorders associated with advanced age.

### 4. DESCRIPTION OF THE FIGURES

FIG. 1 illustrates the comparison of clk-2 eukaryotic homologues (hclk-2: Homo sapiens clk-2: Caenorhabditis elegans tel2p. Saccharomyces cerevisiae Atclk-2: Arabidopsis thaliana).

FIG. 2 illustrates the comparison of clk-2 animal homologues (D.m.: Drosophila melanogaster, H.s.: Homo sapiens C.e.: Caenorhabditis elegans).

FIG. 3 illustrates the comparison of clk-2 vertebrate homologues (H.s.: Homo sapiens, M.m.: Mus musculus, S.s.: Sus scrofa).

FIG. 4 illustrates the comparison of clk-2 plant homologues (A.t.: Arabidopsis thaliana, G.m.: Glycine max, O.s.: Oryza sativa).

FIG. 5 illustrates the expression pattern of clk-2. The spatial and temporal expression pattern of the gene clk-2 was determined by analyzing transcript and protein levels by northern blot and western blot, respectively. (A) The level of clk-2 mRNA (upper panel) appears uniform throughout pre-adult development and increases in adulthood (E, embryos; L1-L4, larval stages; A, adult; glp-4, adult glp-4(bn2ts) mutants at 25° C.). The level of clk-2 protein (lower panel) is similar at all stages including adults. (B) clk-2 mRNA (upper panel) and protein levels (lower panel) in mutant backgrounds glp-4(bn2ts), fem-3(q2ots) and fem-2(b245ts). (C) clk-2 protein levels in wild type and clk-2 (qm37) mutants at three temperatures (15° C., 20° C., and 25° C.). The level of clk-2 is greatly reduced in the mutant, but does not change as a function of temperature in either the wild type or the mutant. Worms were raised at 20° C. except when specified otherwise.

FIG. 6 illustrates the telomere-lengthening phenotype of clk-2(qm37) mutants at different temperatures. The length of a heterogenous population of telomeres in wild-type and clk-2 mutants was examined by Southern blotting. C. elegans were grown at three different temperatures, (A) 18° C., (B) 20° C., and (C) 25° C. Two lanes are shown for each genotype and each temperature. The length of individual telomeres (D) XL and (E) IVL was examined by Southern blotting with a probe specific to the particular telomere. Strain MQ691 carries an extrachromsomal array expressing wild-type clk-2 in a clk-2(qm37) chromosomal background. Strain MQ931 was derived from MQ691 and has lost the extrachromosomal array.

FIGS. 7A-C illustrate the level of clk-2 protein expression in C. elegans expressing clk-2(qm37) suppressors. Level of clk-2 protein was determined by immunoblot analysis of mixed-stage C. elegans population grown at (A) 15° C., (B) 20° C., and (C) 25° C. Each population tested carried either wild type clk-2, clk-2(qm37), clk-2(qm37-A828T), clk-2(qm37-A828V), or clk-2(qm37-S859N). MQ742, MQ743, MQ744, MQ745, MQ1039, MQ1037 correspond to clk-2(qm37-A828T); MQ746 corresponds to clk-2(qm37-A828V); MQ1038 corresponds to clk-2(qm37-S859N); N2 corresponds to wild type clk-2. Tubulin was used as a loading control.

FIG. 8 illustrates the level of clk-2 protein expression in C. elegans expressing A828T suppressors. Level of clk-2(qm37-A828T) protein was determined by immunoblot analysis of mixed-stage C. elegans population grown at 15° C., 20° C., or 25° C. An inverse relationship was found between temperature and protein level. Tubulin was used as a loading control.

FIGS. 9A-B illustrates immunoblot analysis of hclk-2 expression in SK-HEP-1 cells. (A) Overexpression of hclk-2. Cell extracts were prepared from SK-HEP-1 cells expressing full length hclk-2 from a retroviral vector, or cells infected with the empty vector control, and reacted with the anti-hclk-2 antibody. (B) Cell extracts were prepared from SK-HEP-1 cells treated with hclk-2-siRNA, luciferase-siRNA or buffer for the indicated time (numbers above the lanes indicate the day of culture; cells were transfected on day 1). The expression of hclk-2 was specifically reduced by hclk-2 sequence-specific siRNA, but not by non-specific siRNA directed against firefly luciferase or by buffer alone. The immunoblots were probed with an anti-α-actin antibody to control for equal loading of total protein (50 μg in each lane).

FIGS. 10A-B illustrate the affect of hclk-2 expression on growth rate of SK-HEP-1 cells. (A) SK-HEP-1 cells overexpressing hclk-2 and control cells were plated at a density of 1×10⁵/well in a 6-well dish. At the indicated time (in days), cells were harvested and the number of cells were counted using a hemocytometer. The means of triplicate experiments are shown. (B) SK-HEP-1 cells were plated at a density of 1.0×10⁵/well in a 6-well dish and treated by siRNA (to inhibit hclk-2 expression) or buffer the next day (day 1) at a density of about 1.5×10⁵/well. Cell counts were done as above.

FIG. 11 illustrates the affect of hclk-2 overexpression on sensitivity to menadione, t-butyl hydroperoxide and hydroxyurea of SK-HEP-1 cells. SK-HEP-1 cells overexpressing hclk-2 and control cells were seeded at 1×10⁵/well in a 6-well dish and were treated with γ-rays or apoptosis-inducing compounds for various lengths of time (see Table 7). Cells were then trypsinized, diluted and stained with 0.2% trypan blue. Cell viability was expressed as the percentage of cells excluding trypan blue. The data shown are the means of triplicate experiments. When cells were treated with menadione, t-butyl hydroperoxide or hydroxyurea, the viability of SK-HEP-1 cells overexpressing hclk-2 (44%, 30%, 40% respectively) was dramatically lower than that of the control cells (70%, 63%, 76%), respectively.

FIG. 12 illustrates the affect of hclk-2 overexpression on telomere length in SK-HEP-1 cells. DNA was isolated from the cells at the indicated PD and subjected to Southern blot analysis. The mean length of the telomeric restriction fragments in the overexpressing cells gradually became longer (from ˜4 to 7 kb) during the prolonged culture (138 population doublings), while it was remained unchanged in the control cells (˜4 kb).

FIGS. 13A-D illustrate hclk-2 subcellular distribution by immunofluorescence. (A) SK-HEP-1 cells and (B) HT-1080 cells stably infected with pLXSH-hclk-2 were stained for hclk-2 using an anti-hclk-2 antibody. In both cases, the hclk-2 signal can be seen throughout the cell. In the SK-HEP-1 cell shown, there is relatively weaker staining of the nucleus but this was not the case in all cells. In the HT-1080 cell shown, there appears to be relatively intense peri-nuclear staining (arrows) but again, this was not the case in all cells. HT-1080 cells transiently transfected with a construct expressing hclk-2 fused to the ornithine transcarbamylase (OCT) mitochondrial targeting sequence were stained for (C) hclk-2 using the anti-hclk-2 antibody and for (D) mitochondria using Mitotracker Red CMXRos, a dye that is specifically taken up by mitochondria.

FIGS. 14A-B illustrate hclk-2 subcellular distribution by immunoblot. (A) Subcellular fractions from SK-HEP-1 cells and SK-HEP-1 cells overexpressing hclk-2 were analyzed by immunoblotting. hclk-2 is found at similar levels in all fractions. The fractions from the SK-HEP-1 cells were also characterized with mouse monoclonal antibodies against human cytochrome c (mitochondrial marker: heavy-membrane fraction), p300 (nuclear marker) and tubulin (cytosolic marker). As expected, the tubulin signal is mostly in the cytosolic (soluble) fraction, the P300 signal appears to be exclusively present in the nuclear fraction and the cytochrome c signal is mostly present in the heavy-membrane fraction. (B) Nuclear, light-membrane, and heavy-membrane fractions from SK-HEP-1 cells overexpressing hclk-2 were extracted with alkaline sodium carbonate and subjected to immunoblotting using the anti-hclk-2 antibody. In all three compartments, hclk-2 is largely associated with the pellet, indicating that hclk-2 in these compartments is tightly associated with the membrane. An antibody against human cytochrome c, a soluble heavy membrane protein marker, was used as control.

FIGS. 15A-C illustrate mclk-2 tissue distribution by immunoblot. Protein was extracted from adult mouse tissue and immunoblot analysis was performed using anti-mclk-2 polyclonal antibody 3115. Results are shown for three different adult mice (A) (B), and (C).

FIGS. 16A-D illustrate that the anti-mclk-2 polyclonal antibody specifically recognizes mclk-2. Competition experiments were carried out in order to confirm that the 130 kDa band corresponds to mclk-2. The anti-mclk-2 polyclonal antibody 3115 was pre-absorbed with the GST-clk-2 fusion protein (pCB74) used as antigen to generate the polyclonal antibody or with an unrelated GST-fusion (GST-clk-1) prior to immunoblotting protein extracts from (A) brain and heart tissue, (B) kidney and liver tissue, (C) lung and muscle tissue, and (D) spleen and stomach tissue. The 130 kDa band disappears only upon preabsorption with the GST-clk-2 fusion protein indicating that this band corresponds to mclk-2. The ˜60 kDa band disappears after pre-absorption of the antibody with either GST fusion protein, therefore it is not specific to mclk-2.

FIG. 17 illustrates that mclk-2 is a phosphoprotein. Immunoblot analysis using the anti-mclk-2 polyclonal antibody 3115 was conducted on tissue extracts. (A) Brain, heart, kidney or liver tissue extracts were incubated with calf intestinal phosphatase (CIP) to see if mclk-2 was phosphorylated. After treatment with CIP, a shift in mclk-2 mobility was seen. Control reactions included non-CIP treated extracts and extracts incubated with the CIP together with phosphatase inhibitors. (B) Brain, heart, kidney or liver tissue extracts were incubated with ATP to see if kinase activity was present in the tissue extracts. No shift in mobility was seen. Negative control reactions included UTP instead of ATP.

FIG. 18 illustrates that mclk-2 can be further dephosphorylated with increased CIP. Immunoblot analysis using the anti-mclk-2 polyclonal antibody 3115 was conducted on tissue extracts from two different adult mice. (A) heart or (B) liver tissue extract was incubated with increased CIP for longer incubation times. After treatment with CIP, a shift in mclk-2 mobility was seen. (C) Liver tissue extract was denatured prior to treatment with CIP. Both upper and the lower bands migrate significantly lower with this treatment. Control reactions included non-CIP treated extracts and extracts incubated with the CIP together with phosphatase inhibitors.

### 5. DETAILED DESCRIPTION OF THE INVENTION

The present invention relates to nucleotide sequences of clk-2 genes, particularly human clk-2, and amino acid sequences of their encoded proteins, as well as derivatives (e.g., fragments) and analogs thereof.

While clk-2 genes had previously been reported, and a mutant of clk-2 in C. elegans had been characterized, the results in human cells described herein reveal, for the first time, that human clk-2 overexpression increases telomere length, increases cell growth rate, and increases apoptosis in response to oxidative stress or DNA synthesis inhibition. Decreased expression of the human clk-2 decreases telomere length, decreases cell growth rate, and decreases apoptosis in response to oxidative stress or DNA synthesis inhibition. Based on these new observations, the invention provides novel uses of the nucleic acid molecules and polypeptides of the invention as drugs, as drug targets in methods for drug screening, and as a lead to identify other drug targets.

In one embodiment, the invention provides nucleic acids hybridizable to or complementary to clk-2 nucleotide sequences. The invention also provide clk-2 fragments, derivatives and analogs which are functionally active, i.e., they are capable of displaying one or more known functional activities associated with a full-length wild-type clk-2 protein. Such functional activities include but are not limited to antigenicity, i.e., an ability to bind or compete with clk-2 for binding to an anti-clk-2 antibody; immunogenicity, i.e, an ability to generate antibody which binds to clk-2, and biological activities, such as modulation of growth, telomere length, and apoptosis. Methods of production of the clk-2 proteins, derivatives and analogs, e.g., by recombinant means, are also provided. In various embodiments, the preferred clk-2 gene or protein is of human origin.

In another embodiment, the invention provides methods for the identification of agents which modulate the expression of clk-2 gene or the activity of clk-2 protein in cells that are involved in clk-2 related disorders and processes relevant to cancer, neurodegenerative disorders, autoimmune disorders, and aging. Accordingly, the methods of the invention encompass methods for identifying clk-2 agonists, antagonists, or their corresponding inhibitors. clk-2 agonists include, but are not limited to, small molecules (e.g., less than 500 daltons) that bind a clk-2 polypeptide, antibodies directed to a clk-2 polypeptide, and other compounds that interact with a clk-2 polypeptide or a clk-2 gene to enhance its activity or expression. clk-2 antagonists include, but are not limited to, antibodies to clk-2 polypeptides, clk-2 antisense oligonucleotides, clk-2 ribozymes, clk-2 triple-helix molecules, molecules that inhibit binding of regulatory proteins to regulatory regions of a clk-2 gene or otherwise inhibit clk-2 expression, and other small molecules that bind a clk-2 polypeptide, or otherwise inhibit clk-2 gene product activity. Also encompassed are inhibitors of clk-2 agonists and inhibitors of clk-2 antagonists.

Antibodies to clk-2, and clk-2 derivatives and analogs, are provided. Intrabodies as well as antibody conjugates are additionally provided.

In yet another embodiment, the invention also relates to methods for identifying genes or proteins as well as other molecules, such as lipids, which interact with clk-2. These molecules, termed “clk-2 binding partners” herein, are defined via their abilities to interact with the clk-2 gene or gene product, especially to achieve one or more activities associated with clk-2 function.

In yet another embodiment, the present invention also relates to therapeutic and diagnostic methods and compositions based on clk-2 proteins and nucleic acids. Therapeutic agents of the invention include but are not limited to clk-2 proteins and analogs and derivatives (including fragments) thereof; antibodies thereto; nucleic acids encoding the clk-2 proteins, analogs, or derivatives; and clk-2 antisense nucleic acids. Methods for targeting these therapeutic agents to specific organs, tissues, cell types, subcellular locations, as well as tumors, metastatic lesions, degenerating neurons, autoimmune lymphocytes, and aged cells, are provided. Animal models, diagnostic methods and screening methods for predisposition to disorders, are also provided by the invention. For example, the methods and compositions described herein can also be used to regulate apoptosis in various types of tissue, such as tumor tissue including, but not limited to tumors that exhibit abnormal telomere length or cell cycle characteristics.

The invention thus provides for treatment of clk-2-related disorders that are associated with decreased apoptosis (e.g., cancer, autoimmune disorders) or decreased telomere length (e.g., rapid aging or advanced age) by administering to a subject in need thereof an effective amount of an agent that promotes clk-2 activity (e.g., clk-2, an agonist of clk-2; nucleic acids that encode clk-2).

The invention also provides methods of treatment of clk-2-related disorders that are associated with increased apoptosis (e.g., neurodegenerative disorders) and increased telomere length (e.g., cancer) such as by administering to a subject in need thereof an effective amount of an agent that antagonizes, inhibits clk-2 function (e.g., antibodies, antisense nucleic acids). The methods and compositions described herein can also be used to affect the rate and effect of aging and other disorders associated with advanced age.

In yet another embodiment, the present invention also relates to a method for increasing a patient's sensitivity to a therapeutic modality, comprising administering to a subject who is receiving, had received or will receive the therapeutic modality, a clk-2 agent of the invention (e.g., nucleic acid, clk-2 polypeptide, clk-2 agonist, clk-2 antagonist, inhibitor of a clk-2 agonist, inhibitor of a clk-2 antagonist). clk-2 genes and related nucleic acids which can be used in the methods of the invention are described in Section 5.1. Methods of use of the clk-2 genes in producing clk-2 proteins, fragments, and derivatives are also described in Section 5.2.

Further, the gene products of clk-2 genes, fragments, and mutants thereof are described in Sections 5.1 and 5.2, antibodies to such gene products are described in Section 5.5. Cell- and animal-based models of clk-2-related disorders are described in Sections 5.4 and 5.6.

Methods for diagnostic evaluation of various clk-2-related disorders, including cancer as well as clk-2-related processes, such as aging, for the identification of subjects exhibiting a predisposition to such disorders, and for monitoring the efficacy of compounds used in clinical trials are described in Section 5.8.

Methods for the identification of compounds which modulate the expression of clk-2 genes or the activity of clk-2 gene products are described in Section 5.6. Methods for the treatment of cancer and aging and other disorders are described in Section 5.8.

Methods and compositions for the treatment and diagnosis of clk-2 related disorders, including, but not limited to, cancer, neurodegenerative disorders, and autoimmune disorders, are also encompassed by the invention.

5.1 Nucleic Acids of the Invention

The present invention relates to nucleotide sequences of clk-2 genes, particularly human clk-2. In addition to the nucleotide sequences of SEQ ID NOs:1, 4, 5, 6, 7, 15, 16, 20, 21, 22, 23, 24, (see Table 1) it will be appreciated that nucleic acids of the invention also encompass variants thereof, including, but not limited to, any fragment, homologue, naturally occurring allele, or mutant thereof. Nucleic acids of the invention also encompass those nucleic acids capable of hybridization to the disclosed nucleic acids under stringent conditions. Nucleic acids of the invention also encompass those nucleic acids capable of encoding the same polypeptides as the disclosed nucleic acids as well as those nucleic acid that can hybridize under stringent conditions to those nucleic acids capable of encoding the same polypeptides as the disclosed nucleic acids. One or more activities of polypeptides encoded by nucleic acids of the invention can vary relative to the activities of the polypeptides encoded by SEQ ID NOs:1, 4, 5, 6, 7, 15, 16, 20, 21, 22, 23, 24.

In one embodiment, nucleic acids that are at least 75%, 80%, 85%, 90%, 95%, 98%, or 99% identical to any one of the nucleotide sequences of SEQ ID NOs: 1, 4, 5, 6, 7, 15, 16, 20, 21, 22, 23, 24, or variants thereof are encompassed by the invention. To determine the percent identity of two nucleic acid sequences, the sequences are aligned for optimal comparison purposes (e.g., gaps can be introduced in the sequence of a first nucleic acid sequence for optimal alignment with a second or nucleic acid sequence). The nucleotides at corresponding nucleotide positions are then compared. When a position in the first sequence is occupied by the same nucleotide as the corresponding position in the second sequence, then the molecules are identical at that position. The percent identity between the two sequences is a function of the number of identical positions shared by the sequences (i.e., % identity=number of identical overlapping positions/total number of positions×100%). In one embodiment, the two sequences are the same length.

The determination of percent identity between two sequences can also be accomplished using a mathematical algorithm. A preferred, non-limiting example of a mathematical algorithm utilized for the comparison of two sequences is the algorithm of Karlin and Altschul, 1990, PNAS 87:2264-2268, modified as in Karlin and Altschul, 1993, PNAS 90:5873-5877. Such an algorithm is incorporated into the NBLAST and XBLAST programs of Altschul et al., 1990, J. Mol. Biol. 215:403. BLAST nucleotide searches can be performed with the NBLAST nucleotide program parameters set, e.g., for score=100, wordlength=12 to obtain nucleotide sequences homologous to a nucleic acid molecules of the present invention. To obtain gapped alignments for comparison purposes, Gapped BLAST can be utilized as described in Altschul et al., 1997, Nucleic Acids Res. 25:3389-3402. Alternatively, PSI-BLAST can be used to perform an iterated search which detects distant relationships between molecules (Id.). When utilizing BLAST, Gapped BLAST, and PSI-Blast programs, the default parameters of the respective programs (e.g., of NBLAST) can be used. Another preferred, non-limiting example of a mathematical algorithm utilized for the comparison of sequences is the algorithm of Myers and Miller, 1988, CABIOS 4:11-17. Such an algorithm is incorporated in the ALIGN program (version 2.0) which is part of the GCG sequence alignment software package. When utilizing the ALIGN program for comparing amino acid sequences, a PAM120 weight residue table, a gap length penalty of 12, and a gap penalty of 4 can be used.

The percent identity between two sequences can be determined using techniques similar to those described above, with or without allowing gaps. In calculating percent identity, typically only exact matches are counted.

In another embodiment, fragments of any of SEQ ID NOs: 1, 4, 5, 6, 7, 15, 16, 20, 21, 22, 23, 24, or variants thereof are encompassed by the invention. The invention features nucleic acid molecules which include a fragment of at least 100, 200, 300, 350, 400, 450, 500, 550, 600, 650, 700, 750, 800, 850, 900, 950, 1000, 1100, 1200, 1300, 1400, 1500, 1600, 1700, 1800, 1900 or 2000 contiguous nucleotides of the nucleotide sequence of any of SEQ ID NOs:1, 4, 5, 6, 7, 15, 16, 20, 21, 22, 23, 24, or variants or complement thereof. In a preferred embodiment, the fragment encompasses at least a portion of the open reading frame.

Those skilled in the art will recognize that nucleic acid sequence polymorphisms that may or may not lead to changes in the encoded amino acid sequence may exist within a population (e.g., the human population). Such genetic polymorphisms may exist among individuals within a population due to natural allelic variation. An allele is one of a group of genes which occur alternatively at a given genetic locus. As used herein, the phrase “allelic variant” refers to a nucleotide sequence which occurs at a given locus or to a polypeptide encoded by the nucleotide sequence. As used herein, a “naturally-occurring” nucleic acid molecule refers to an RNA or DNA molecule having a nucleotide sequence that occurs in nature (e.g., encodes a natural protein). Naturally-occurring allelic variations can typically result in 1-5% variance in the nucleotide sequence of a given gene. Usually naturally occurring variations do not alter or do not substantially alter the functional activity of the encoded polypeptide. Alternative alleles can be identified by sequencing the gene of interest in a number of different individuals. This can be readily carried out by using hybridization probes to identify the same genetic locus in a variety of individuals. Any and all such nucleotide variations and resulting amino acid polymorphisms or variations that are the result of natural allelic variation are intended to be within the scope of the invention. In one embodiment, polymorphisms that are associated with a particular disorder are used as markers to diagnose said disorder.

Moreover, nucleic acid molecules encoding proteins of the invention from other species (homologs), which have a nucleotide sequence which differs from that of the C. elegans or human protein described herein are intended to be within the scope of the invention. Nucleic acid molecules corresponding to natural allelic variants and homologs of a nucleic acid of the invention can be isolated based on their identity to the C. elegans or human nucleic acid molecule disclosed herein using the C. elegans or human nucleic acid, or a portion thereof, as a hybridization probe according to standard hybridization techniques under stringent hybridization conditions.

Accordingly, in another embodiment, an isolated nucleic acid molecule of the invention is at least 25, 50, 100, 200, 300, 400, 500, 600, 700, 800, 900, 1000, 1100, 1200, 1300, 1400, 1500, 1600, 1700, 1800, 1900 or 2000 contiguous nucleotides in length and hybridizes under stringent conditions to the nucleic acid molecule comprising the nucleotide sequence, preferably the coding sequence, of SEQ ID NOs:1, 4, 5, 6, 7, 15, 16, 20, 21, 22, 23, 24, or a complement thereof.

In addition to naturally-occurring allelic variants of a nucleic acid molecule of the invention, the skilled artisan will further appreciate that changes can be introduced by mutation into the nucleotide sequence of a nucleic acid of the invention that may or may not result in changes in the amino acid sequence of the encoded protein, either with or without altering the biological activity of the protein. Such mutant nucleic acids are also encompassed in the invention.

Accordingly, in another embodiment, the invention pertains to nucleic acid molecules encoding a polypeptide of the invention that contain changes in amino acid residues that may or may not be essential for at least one activity. Such polypeptides differ in amino acid sequence from SEQ ID NOs:2, 3, 8, 9, 10, 11, 12, 13, 14, 17, 18, 19, 25, 26, 27, 28, 29, 30, 31, 32, yet retain at least one biological activity.

Another aspect of the invention pertains to nucleic acid molecules that encode polypeptides that include an amino acid sequence that is at least about 30%, 35%, 40%, 45%, 50%, 55%, 60%, 65%, 75%, 85%, 95%, or 98% identical to the amino acid sequence of SEQ ID NOs:2, 3, 8, 9, 10, 11, 12, 13, 14, 17, 18, 19, 25, 26, 27, 28, 29, 30, 31, 32.

As used herein, the term “hybridizes under stringent conditions” is intended to describe conditions for hybridization and washing under which nucleotide sequences at least 60%, 65%, 70%, 75%, 80%, 85%, 90% identical to each other typically remain hybridized to each other. Such stringent conditions are known to those skilled in the art and can be found in, for example, Ausubel, F. M. et al., eds. 1989 Current Protocols in Molecular Biology, vol. 1, Green Publishing Associates, Inc. and John Wiley and Sons, Inc., NY at pages 6.3.1 to 6.3.6 and 2.10.3. A preferred, non-limiting example of stringent hybridization conditions are hybridization in 6× sodium chloride/sodium citrate (SSC) at about 45° C. followed by one or more washes in 0.2×SSC, 0.1% SDS at 50-65° C. Highly stringent conditions such as hybridization to filter-bound DNA in 6×SSC at about 45° C. followed by one or more washes in 0.1×SSC/0.2% SDS at about 60° C. can also be used in the invention.

Therefore, also encompassed in the nucleic acids of the invention are nucleic acid molecules that hybridize under stringent conditions to any one of the sequences of SEQ ID NOs:1, 4, 5, 6, 7, 15, 16, 20, 21, 22, 23, 24 or a complement thereof. Also encompassed by the invention are nucleic acid molecules that hybridize under stringent conditions to a nucleic acid that encodes any one of the sequences of SEQ ID NOs:2, 3, 8, 9, 10, 11, 12, 13, 14, 17, 18, 19, 25, 26, 27, 28, 29, 30, 31, 32, or a derivative thereof.

5.1.1 Methods for Generating Mutants

Mutant polypeptides of the invention can be generated for use in the methods of the invention. In particular, such mutant polypeptides can be expressed in C. elegans or in recombinant cells for use in screening for agents of the invention (see Section 5.6). Preferably, a mutant polypeptide exhibits altered activity in at least one function displayed by the wild type polypeptide. The altered activity of the mutant polypeptide can be a decrease (e.g., loss-of-function mutation) or increase (e.g., gain-of-function mutation) in activity. As used herein, the phrase “loss-of-function mutation” refers to a mutation such that the mutant polypeptide has decreased activity. The decreased activity may be present in each of the functions/activities of the polypeptide or may present in fewer than all of the functions/activities of the polypeptide. A loss-of-function mutation can be a complete or partial loss-of-function. As used herein, the phrase “gain-of-function mutation” refers to a mutation such that the mutant polypeptide has increased activity. The increased activity may be present in each of the functions/activities of the polypeptide or may present in fewer than all of the functions/activities of the polypeptide.

In one embodiment, a mutant polypeptide that is a variant of a polypeptide of the invention can be assayed for: (1) the ability to form protein-protein interactions with proteins in a signaling pathway of the polypeptide of the invention; (2) the ability to bind a ligand of the polypeptide of the invention; or (3) the ability to bind to an intracellular target protein of the polypeptide of the invention.

In another embodiment, a mutant polypeptide can be assayed for the ability to function in similar ways as the polypeptide of the invention. In a specific embodiment wherein the polypeptide of the invention is clk-2, mutant clk-2 polypeptides can be assayed for the functional properties they share with wild type clk-2. For embodiments where mutant clk-2 polypeptides are expressed in C. elegans, the mutant clk-2 can be assayed for the ability to modulate telomere length, length of life, length of embryonic and post-embryonic development, frequency of defecation cycles, pharyngeal pumping rate, self-brood size, peak egg-laying rate, proportion of dead eggs, and larvae among the progeny of gamma-irradiated animal as compared to wild type clk-2. In embodiments where mutant clk-2 polypeptides are expressed in recombinant cells, the mutant clk-2 can be assayed for the ability to possess the ability to modulate telomere length, cell growth rate, and apoptosis in response to oxidative stress or DNA synthesis inhibition relative as compared to wild type clk-2.

Any method known in the art can be used for generating mutant polypeptides. For example, mutant nucleic acid molecules can be generated in live animals or cells (e.g. EMS chemical deletion mutagenesis, Tc1 transposon insertion mutagenesis, ect.) or biochemically (e.g., molecular evolution techniques such as site directed mutagenesis).

5.1.1.1 EMS Chemical Deletion Mutagenesis

Ethyl methanesulfonate (EMS) is a commonly-used chemical mutagen for creating loss-of-function mutations in genes-of-interest in C. elegans. Approximately 13% of mutations induced by EMS are small deletions. With the methods described herein, there is approximately a 95% probability of identifying a deletion-of-interest by screening 4×10⁶ EMS-mutagenized genomes. After mutagenesis, mutant C. elegans are further screened to identify those mutations that are in a gene encoding a polypeptide of the invention. Briefly, this procedure involves creating a library of several million mutagenized C. elegans which are distributed in small pools in 96-well plates, each pool composed of approximately 400 haploid genomes. A portion of each pool is used to generate a corresponding library of genomic DNA derived from the mutagenized nematodes. The DNA library is screened with a PCR assay to identify pools that carry genomes with deletions-of-interest, and mutant worms carrying the desired deletions are recovered from the corresponding pools of the mutagenized animals. Although EMS is a preferred mutagen to generate deletions, other mutagens can be used that also provide a significant yield of deletions, such as X-rays, gamma-rays, diepoxybutane, formaldehyde and trimethylpsoralen with ultraviolet light.

Nematodes may be mutagenized with EMS using any procedure known to one skilled in the art, such as the procedure described by Sulston and Hodgkin (1988, pp. 587-606, in The Nematode Caenorhabditis elegans, Wood, Ed., Cold Spring Harbor Laboratory Press, Cold Spring Harbor, N.Y.). Following exposure to the mutagen, nematodes are dispensed into petri dishes, incubated one to two days, and embryos isolated by hypochlorite treatment (Id.) Embryos are allowed to hatch and L1 larvae are collected following overnight incubation. The larvae are distributed in petri plates at an average density of 200 animals per plate and incubated for 5 to 7 days until just starved. A sample of nematodes is collected from each plate by washing with a solution of distilled water, and the nematodes washed from each plate are placed in one well of a 96-well plate. Worms are lysed and DNA stored at −80° C. until further analysis. Live nematodes from each plate are aliquoted into tubes within racks for storage at −80° C., such that the physical arrangement of tubes of live animals is the same as the arrangement of corresponding DNA lysates in the 96-well plates.

A pooling strategy is used to allow efficient PCR screening of the DNA lysates. The pools are made from each 96-well plate by mixing 10 μl of lysate from 8 wells comprising each column of wells in a plate. The pooled lysates for each column are used for screening with PCR. PCR primers are designed for each locus-of-interest to be about 1.5 to 12 kb apart, depending on the size of the locus, such that deletions encompassing the entire coding regions of nucleic acid molecules of the invention can be detected following a previously-described procedure (see Plasterk, 1995, Methods in Cell Biology 48:59-80). For each region, two sets of primer pairs are chosen for carrying out a nested PCR strategy such that an outside set is used for the first round of PCR and an inside set is used for the second round of PCR. The second round of PCR is performed to achieve greater specificity in the reaction.

Products of the second round of PCR may be analyzed by electrophoresis in agarose or acrylamide gels. If a potential deletion product is observed in at least one of the two reactions, two rounds of PCR are performed as described above on lysates from each individual well derived from the column corresponding to the positive pool. This results in the identification of a positive “address,” i.e., a specific well within an individual plate, containing a deletion mutant. The positive address is re-tested in quadruplicate using two rounds of PCR as described above, and the product is gel purified and sequenced directly to confirm the presence of the desired deletion.

Once a positive address has been identified and confirmed by sequence analysis, approximately 300 individual worms from the relevant plate are cloned onto separate, fresh plates. When F1 animals are present on the plate, the parent nematodes are placed into buffer and lysed as described above. The same primer pairs and cycling conditions used to identify the deletion are used to perform PCR on these animals. Once a single animal carrying the deletion has been identified, its progeny are cloned and examined using the same conditions described above, until a homozygous population of deletion animals is obtained.

5.1.1.2 Tc1 Transposon Insertion Mutagenesis

The transposable element Tc1 may also be used as a mutagen in C. elegans since insertion of the transposable element into a gene-of-interest can result in the inactivation of gene function. After mutagenesis, mutant C. elegans are further screened to identify those mutations that are in a gene encoding a polypeptide of the invention. Starting with a strain that contains a high copy number of the Tc1 transposable element in a mutator background (i.e., a strain in which the transposable element is highly mobile), a Tc library containing approximately 3,000 individual cultures is created as previously described (see e.g., Zwaal et al., 1993, PNAS 90:7431-7435; Plasterk, 1995, “Reverse Genetics: From Gene Sequence to Mutant Worm”, in Caenorhabditis elegans: Modem Biological Analysis of an Organism (Epstein and Shakes, Eds.) pp. 59-80.). The library is screened for Tc1 insertions in the region of interest using the polymerase chain reaction with one set of primers specific for Tc1 sequence and one set of gene-specific primers (e.g., primers for clk-2). Because Tc1 exhibits a preference for insertion within introns, it is sometimes necessary to carry out a secondary screen of populations of insertion animals for imprecise excision of the transposable element, which can result in deletion of part or all of the gene of interest (generally, 1-2 kb of genomic sequence is deleted). The screen for Tc1 deletions is performed and deletion animals are recovered in the same manner as for the EMS screen described above.

5.1.1.3 Molecular Evolution Techniques

Mutant polypeptides can be created by introducing one or more nucleotide substitutions, additions or deletions into the nucleotide sequence of the nucleic acids of the invention (e.g., clk-2), such that one or more amino acid substitutions, additions or deletions are introduced into the encoded protein. Mutations can be introduced by standard techniques such as directed molecular evolution techniques (see generally Arnold, 1993, Curr. Opinion Biotechnol. 4:450-455); e.g., site-directed mutagenesis (see e.g., Kunkel, 1985, PNAS, 82:488-492; Oliphant et al., 1986, Gene 44:177-183); oligonucleotide-directed mutagenesis (see e.g., Reidhaar-Olson et al., 1988, Science 241:53-57); chemical mutagenesis (see e.g., Eckert et al., 1987, Mutat. Res. 178: 1-10); error prone PCR (see e.g., Caldwell & Joyce, 1992, PCR Methods Applic. 2:28-33); cassette mutagenesis (see e.g., Arkin et al., PNAS, 1992, 89:7871-7815); DNA shuffling methods (see e.g., Stemmer et al., 1994, PNAS, 91:10747-10751; U.S. Pat. Nos. 5,605,793; 6,117,679; 6,132,970; 5,939,250; 5,965,408; 6,171,820).

In one embodiment, particular nucleotide sequences or positions of the nucleic acid of the invention are targeted for mutation. Such targeted mutations can be introduced at any position in the nucleic acid. For example, one can make nucleotide substitutions leading to amino acid substitutions at “non-essential” or “essential” amino acid residues. A “non-essential” amino acid residue is a residue that can be altered from the wild-type sequence without altering the biological activity, whereas an “essential” amino acid residue is required for at least one biological activity of the polypeptide. For example, amino acid residues that are not conserved or only semi-conserved among homologs of various species may be non-essential for activity. Alternatively, amino acid residues that are conserved among the homologs of various species (e.g., mouse and human) may be essential for activity.

Such targeted mutations can also be made at one or more non-conservative amino acid residues. A “non-conservative amino acid substitution” is one in which the amino acid residue is replaced with an amino acid residue having a dissimilar side chain. Families of amino acid residues having similar side chains have been defined in the art. These families include amino acids with basic side chains (e.g., lysine, arginine, histidine), acidic side chains (e.g., aspartic acid, glutamic acid, asparagine, glutamine), uncharged polar side chains (e.g., glycine, serine, threonine, tyrosine, cysteine), nonpolar side chains (e.g., alanine, valine, leucine, isoleucine, proline, phenylalanine, methionine, tryptophan), β-branched side chains (e.g., threonine, valine, isoleucine) and aromatic side chains (e.g., tyrosine, phenylalanine, tryptophan, histidine).

Alternatively or in addition to non-conservative amino acid residue substitutions, such targeted mutations are made at one or more conservative amino acid residues. A “conservative amino acid substitution” is one in which the amino acid residue is replaced with an amino acid residue having a similar side chain. Following mutagenesis, the encoded protein can be expressed recombinantly and the activity of the protein can be determined.

In another embodiment, mutations can be introduced randomly along all or part of the coding sequence (e.g., by saturation mutagenesis). In certain embodiments, nucleotide sequences encoding other related polypeptides that have similar domains, structural motifs, active sites, or that aligns with a portion of the enzyme gene of the invention with mismatches or imperfect matches, can be used in the mutagenesis process to generate diversity of sequences. It should be understood that for each mutagenesis step in some of the techniques mentioned above, a number of iterative cycles of any or all of the steps may be performed to optimize the diversity of sequences. The above-described methods can be used in combination in any desired order. In many instances, the methods result in a pool of mutant nucleotide sequences or a pool of recombinant host cells comprising mutant nucleotide sequences. The nucleotide sequences or host cells expressing a modified enzyme with the desired characteristics can be identified by screening with one or more assays that are well known in the art. The assays may be carried out under conditions that select for polypeptides possessing the desired physical or chemical characteristics. The mutations in the nucleotide sequence can be determined by sequencing the nucleic acid encoding the mutant polypeptide in the clones.

5.2 Polypeptides of the Invention

The present invention relates to amino acid sequences of clk-2 protein, particularly human clk-2. In addition to the polypeptide sequences of SEQ ID NOs:2, 3, 8, 9, 10, 11, 12, 13, 14, 17, 18, 19, 25, 26, 27, 28, 29, 30, 31, 32, (see Table 1) it will be appreciated that polypeptides of the invention also encompass variants thereof, including, but not limited to, any fragment, derivative, homologue, naturally occurring allele, mutant thereof.

Polypeptides of the invention also encompass those polypeptides that are encoded by any of the nucleic acids described in Section 5.1.

TABLE 1

| | SEQ | | | |
|---|---|---|---|---|
| | ID NO. | Sequence | Species | Abbrev. |
| clk-2 | 1 | cDNA | Caenorhabditis elegans | |
| clk-2 | 2 | protein | Caenorhabditis elegans | |
| clk-2 | 3 | protein | Homo sapiens | hclk-2 |
| clk-2 | 4 | cDNA | Homo sapiens | |
| clk-2 | 5 | cDNA | Mus musculus | mclk-2 |
| clk-2 | 6 | cDNA | Mus musculus | |
| clk-2 | 7 | cDNA | Mus musculus | |
| clk-2 | 8 | protein | Mus musculus | |
| clk-2 | 9 | protein | Mus musculus | |
| clk-2 | 10 | protein | Mus musculus | |
| clk-2 | 11 | protein | Mus musculus | |
| clk-2 | 12 | protein | Sus scrofa | ssclk-2 |
| clk-2 | 13 | protein | Drosophila melanogaster | dclk-2 |
| clk-2 | 14 | protein | Arabidopsis thaliana | aclk-2 |
| clk-2 | 15 | cDNA | Oryza sativa | oclk-2 |
| clk-2 | 16 | cDNA | Oryza sativa | |
| clk-2 | 17 | protein | Oryza sativa | |
| clk-2 | 18 | protein | Oryza sativa | |
| clk-2 | 19 | protein | Oryza sativa | |
| clk-2 | 20 | cDNA | Glycine max | |
| clk-2 | 21 | cDNA | Glycine max | gclk-2 |
| clk-2 | 22 | cDNA | Glycine max | |
| clk-2 | 23 | cDNA | Glycine max | |
| clk-2 | 24 | cDNA | Glycine max | |
| clk-2 | 25 | protein | Glycine max | |
| clk-2 | 26 | protein | Glycine max | |
| clk-2 | 27 | protein | Glycine max | |
| clk-2 | 28 | protein | Glycine max | |
| clk-2 | 29 | protein | Glycine max | |
| clk-2 | 30 | protein | Glycine max | |
| clk-2 | 31 | mutant protein | Caenorhabditis elegans | |
| clk-2 | 32 | protein | Saccharomyces cerevisiae | sclk-2 |

As used herein, the term “derivative” refers to a polypeptide that comprises an amino acid sequence of a polypeptide of the invention (e.g., clk-2) which has been altered by the introduction of amino acid residue substitutions, deletions or additions. Derivative polypeptides may or may not possess residues that have been modified, i.e, by the covalent attachment of any type of molecule to the polypeptide. For example, but not by way of limitation, a derivative polypeptide of the invention may be modified, e.g., by glycosylation, acetylation, pegylation, phosphorylation, amidation, derivatization by known protecting/blocking groups, proteolytic cleavage, linkage to a cellular ligand or other protein, etc. A derivative of a polypeptide of the invention may be modified by chemical modifications using techniques known to those of skill in the art, including, but not limited to specific chemical cleavage, acetylation, formylation, metabolic synthesis of tunicamycin, etc. Furthermore, a derivative of a polypeptide of the invention may contain one or more non-classical amino acids.

In one embodiment, a polypeptide derivative is a functionally active derivative and possesses at least one, preferably more, similar or identical function as a polypeptide of the invention described herein such as, but not limited to, any one of the following: binding to antibodies that are raised against the wild type polypeptide, altering telomere length, altering cellular growth rate, altering sensitivity to apoptosis (especially apoptosis caused by oxidative stress or DNA synthesis inhibition), altering length of life span of an organism, and altering developmental rate of an organism. In another embodiment, a derivative of a polypeptide of the invention has an increased or decreased activity in one or more of the foregoing functions when compared to an unaltered polypeptide. Other altered activities include, but are not limited to, resistance to proteolysis or increased ability to cross a cell membrane.

One group of derivatives are phosphorylated polypeptides of the invention, wherein one or more serine, threonine, and/or tyrosine residues are phosphorylated. Preferred derivatives of mouse and human hclk2 polypeptide can be phosphorylated at one or more of the following residues which are conserved among the two clk-2 polypeptides: serine residue(s) in hclk-2 at 20, 21, 114, 326, 414, 485, 487, 491, 605; serine residue(s) in mclk-2 at 20, 21, 114, 326, 414, 486, 488, 492, 606; threonine residue(s) in hclk-2 at 300, 523, 524, 584, 673; threonine residue(s) in mclk-2 at 300, 524, 525, 585, 674; tyrosine residue(s) in hclk-2 at 536; and tyrosine residue in mclk-2 at 537. Other putative phosphorylation sites that are conserved between human and mouse clk2 include but are not limited to: serine residues at 5, 71, 397, 545, 553, 651, 671, 677, 772; threonine at 145, 312, 741; and tyrosine at 498, 514, 765 (using hclk-2 residue numbers).

In one embodiment, polypetides that are at least 75%, 80%, 85%, 90%, 95%, 98%, or 99% identical to any one of the polypeptide sequences of SEQ ID NOs:2, 3, 8, 9, 10, 11, 12, 13, 14, 17, 18, 19, 25, 26, 27, 28, 29, 30, 31, 32, or variants thereofare encompassed by the invention. The degree of similarity (or percent identity) can be calculated by methods disclosed in Section 5.1 (with the caveat that NBLAST is not used in BLAST amino acid searches, rather XBLAST is used with program parameters set, e.g., to score-50, wordlength=3).

In another embodiment, fragments of any of SEQ ID NOs:2, 3, 8, 9, 10, 11, 12, 13, 14, 17, 18, 19, 25, 26, 27, 28, 29, 30, 31, 32 or variants thereof are encompassed by the invention. The invention features polypeptides which include a fragment of at least 5, 10, 15, 20, 25, 40, 50, 60, 70, 80, 90, 100, 125, 150, 175, 200, 250, 300, 350, 400, 450, 500, 550, 600, 650, 700, 750, 800 consecutive amino acid residues of the amino acid sequence of any one of the polypeptides of SEQ ID NOs:2, 3, 8, 9, 10, 11, 12, 13, 14, 17, 18, 19, 25, 26, 27, 28, 29, 30, 31, 32 or variant thereof. A fragment of a polypeptide of the invention may or may not be immunogenic and/or antigenic. Preferably, a fragment of a polypeptide of the invention retains some level of function in at least one activity of the full length polypeptide.

Accordingly, in another embodiment, an isolated polypeptide of the invention is at least 25, 50, 100, 200, 300, 400, 500, 600, 700, 800, 900, 1000 contiguous amino acids in length and the nucleic acid that encodes such a polypeptide of the invention hybridizes under stringent conditions to the nucleic acid that encodes any one of SEQ ID NOs:2, 3, 8, 9, 10, 11, 12, 13, 14, 17, 18, 19, 25, 26, 27, 28, 29, 30, 31, 32 or a variant thereof.

In addition to naturally-occurring allelic variants of a polypeptide of the invention, the skilled artisan will further appreciate that changes can be introduced by mutation into the nucleotide sequence of a nucleic acid encoding a polypeptide of the invention that may or may not result in changes in the biological activity of the protein. Such mutant polypeptides are also encompassed in the invention.

Accordingly, in another embodiment, the invention pertains to polypeptides that contain changes in amino acid residues that may or may not be essential for at least one activity. Such polypeptides differ in amino acid sequence from SEQ ID NOs:2, 3, 8, 9, 10, 11, 12, 13, 14, 17, 18, 19, 25, 26, 27, 28, 29, 30, 31, 32, yet retain at least one biological activity.

Another aspect of the invention pertains to polypeptides that are immunospecifically bound to by an antibody that immunospecifically binds to any one of the polypeptides of SEQ ID NOs:2, 3, 8, 9, 10, 11, 12, 13, 14, 17, 18, 19, 25, 26, 27, 28, 29, 30, 31, 32.

5.3 Recombinant Expression

Another aspect of the invention pertains to vectors, preferably expression vectors, comprising a nucleic acid of the invention, or a variant thereof. As used herein, the term “vector” refers to a polynucleotide capable of transporting another nucleic acid to which it has been linked. One type of vector is a “plasmid”, which refers to a circular double stranded DNA loop into which additional DNA segments can be introduced. Another type of vector is a viral vector, wherein additional DNA segments can be introduced into the viral genome.

Certain vectors are capable of autonomous replication in a host cell into which they are introduced (e.g., bacterial vectors having a bacterial origin of replication and episomal mammalian vectors). Other vectors (e.g., non-episomal mammalian vectors) are integrated into the genome of a host cell upon introduction into the host cell, and thereby are replicated along with the host genome. In general, expression vectors of utility in recombinant DNA techniques are often in the form of plasmids (vectors). However, the invention is intended to include such other forms of expression vectors, such as viral vectors (e.g., replication defective retroviruses, adenoviruses and adeno-associated viruses).

The recombinant expression vectors of the invention comprise a nucleic acid of the invention in a form suitable for expression of the nucleic acid in a host cell. This means that the recombinant expression vectors include one or more regulatory sequences, selected on the basis of the host cells to be used for expression, which is operably associated with the polynucleotide to be expressed. Within a recombinant expression vector, “operably associated” is intended to mean that the nucleotide sequence of interest is linked to the regulatory sequence(s) in a manner which allows for expression of the nucleotide sequence (e.g., in an in vitro transcription/translation system or in a host cell when the vector is introduced into the host cell). The term “regulatory sequence” is intended to include promoters, enhancers and other expression control elements (e.g., polyadenylation signals). Such regulatory sequences are described, for example, in Goeddel, Gene Expression Technology: Methods in Enzymology, (1990) Academic Press, San Diego, Calif., p. 185. Regulatory sequences include those which direct constitutive expression of a nucleotide sequence in many types of host cell and those which direct expression of the nucleotide sequence only in certain host cells (e.g., tissue-specific regulatory sequences). It will be appreciated by those skilled in the art that the design of the expression vector can depend on such factors as the choice of the host cell to be transformed, the level of expression of protein desired, etc. The expression vectors of the invention can be introduced into host cells to thereby produce proteins or peptides, including fusion proteins or peptides, encoded by nucleic acids as described herein.

The recombinant expression vectors of the invention can be designed for expression of a polypeptide of the invention in prokaryotic (e.g., E. coli) or eukaryotic cells (e.g., insect cells using baculovirus expression vectors, yeast cells, C. elegans cells, or mammalian cells). Suitable host cells are discussed further in Goeddel, supra. Alternatively, the recombinant expression vector can be transcribed and translated in vitro, for example using T7 promoter regulatory sequences and T7 polymerase.

Expression of proteins in prokaryotes is most often carried out in E. coli with vectors comprising constitutive or inducible promoters directing the expression of either fusion or non-fusion proteins. Fusion vectors add a number of amino acids to a protein encoded therein, usually to the amino terminus of the recombinant protein. Such fusion vectors typically serve at least three purposes: 1) to increase expression of recombinant protein; 2) to increase the solubility of the recombinant protein; and/or 3) to aid in the purification of the recombinant protein by acting as a ligand in affinity purification. Often, in fusion expression vectors, a proteolytic cleavage site is introduced at the junction of the fusion moiety and the recombinant protein to enable separation of the recombinant protein from the fusion moiety subsequent to purification of the fusion protein. Such enzymes, and their cognate recognition sequences, include Factor Xa, thrombin and enterokinase. Typical fusion expression vectors include pGEX (Pharmacia Biotech Inc; Smith and Johnson, 1988, Gene 67:31-40), pMAL (New England Biolabs, Beverly, Mass.) and pRIT5 (Pharmacia, Piscataway, N.J.) which fuse glutathione S-transferase (GST), maltose E binding protein, or protein A, respectively, to the target recombinant protein.

Examples of suitable inducible non-fusion E. coli expression vectors include pTrc (Amann et al., 1988, Gene 69:301-315) and pET 11d (Studier et al., Gene Expression Technology: Methods in Enzymology 185, Academic Press, San Diego, Calif. (1990) p. 60-89). Target gene expression from the pTrc vector relies on host RNA polymerase transcription from a hybrid trp-lac fusion promoter. Target gene expression from the pET 11d vector relies on transcription from a T7 gn10-lac fusion promoter mediated by a coexpressed viral RNA polymerase (T7 gn1). This viral polymerase is supplied by host strains BL21(DE3) or HMS174(DE3) from a resident λ prophage harboring a T7 gnl gene under the transcriptional control of the lacUV 5 promoter.

One strategy to maximize recombinant protein expression in E. coli is to express the protein in a host bacteria with an impaired capacity to proteolytically cleave the recombinant protein (Gottesman, Gene Expression Technology: Methods in Enzymology 185, Academic Press, San Diego, Calif. (1990) p. 119-128). Another strategy is to alter the sequence of the nucleic acid to be inserted into an expression vector so that the individual codons for each amino acid are those preferentially utilized in E. coli (Wada et al., 1992, Nucleic Acids Res. 20:2111-2118). Such alteration of polynucleotides of the invention can be carried out by standard DNA synthesis techniques.

In another embodiment, the expression vector is a yeast expression vector. Examples of vectors for expression in yeast S. cerevisiae include pYepSec1 (Baldari et al., 1987, EMBO.J 6:229-234), pMFa (Kurjan and Herskowitz, 1982, Cell 30:933-943), pJRY88 (Schultz et al., 1987, Gene 54:113-123), pYES2 (Invitrogen Corp., San Diego, Calif.), and pPicZ (Invitrogen Corp., San Diego, Calif.).

Alternatively, the expression vector is a baculovirus expression vector. Baculovirus vectors available for expression of proteins in cultured insect cells (e.g., Sf 9 cells) include the pAc series (Smith et al., 1983, Mol. Cell Biol. 3:2156-2165) and the pVL series (Lucklow and Summers, 1989, Virology 170:31-39).

In yet another embodiment, a nucleic acid of the invention is expressed in mammalian cells using a mammalian expression vector. Examples of mammalian expression vectors include pCDM8 (Seed, 1987, “An LFA-3 cDNA encodes a phospholipid-linked membrane protein homologous to its receptor CD2”, Nature: 840-842) and pMT2PC (Kaufman et al., 1987, EMBO J. 6:187-193). When used in mammalian cells, the expression vector's control functions are often provided by viral regulatory elements. For example, commonly used promoters are derived from polyoma, Adenovirus 2, cytomegalovirus and Simian Virus 40. For other suitable expression systems for both prokaryotic and eukaryotic cells see chapters 16 and 17 of Sambrook et al. 1990, Molecular Cloning, A Laboratory Manual, 2d Ed., Cold Spring Harbor Laboratory, Cold Spring Harbor, N.Y.

In another embodiment, the recombinant mammalian expression vector is capable of directing expression of the nucleic acid preferentially in a particular cell type (e.g., tissue-specific regulatory elements are used to express the nucleic acid). Tissue-specific regulatory elements are known in the art. Non-limiting examples of suitable tissue-specific promoters include the albumin promoter (liver-specific; Pinkert et al., 1987, Genes Dev. 1:268-277), lymphoid-specific promoters (Calame and Eaton, 1988, Adv. Immunol. 43:235-275), in particular promoters of T cell receptors (Winoto and Baltimore, 1989, EMBO J. 8:729-733) and immunoglobulins (Banerji et al., 1983, Cell 33:729-740; Queen and Baltimore, 1983, Cell 33:741-748), neuron-specific promoters (e.g., the neurofilament promoter; Byrne and Ruddle, 1989, Proc Natl Acad. Sci. 86:5473-5477), pancreas-specific promoters (Edlund et al., 1985, Science 230:912-916), and mammary gland-specific promoters (e.g., milk whey promoter; U.S. Pat. No. 4,873,316 and European Application Publication No. 264,166). Developmentally-regulated promoters are also encompassed, for example the murine hox promoters (Kessel and Gruss, 1990, Science 249:374-379) and the α-fetoprotein promoter (Campes and Tilghman, 1989, Genes Dev. 3:537-546).

The invention further provides a recombinant expression vector comprising a polynucleotide of the invention cloned into the expression vector in an antisense orientation. That is, the DNA molecule is operably associated with a regulatory sequence in a manner which allows for expression (by transcription of the DNA molecule) of an RNA molecule which is antisense to the mRNA encoding a polypeptide of the invention. Regulatory sequences operably associated with a nucleic acid cloned in the antisense orientation can be chosen which direct the continuous expression of the antisense RNA molecule in a variety of cell types, for instance viral promoters and/or enhancers, or regulatory sequences can be chosen which direct constitutive, tissue specific or cell type specific expression of antisense RNA. The antisense expression vector can be in the form of a recombinant plasmid, phagemid or attenuated virus in which antisense nucleic acids are produced under the control of a high efficiency regulatory region, the activity of which can be determined by the cell type into which the vector is introduced.

In another embodiment, the expression characteristics of an endogenous gene corresponding to a nucleic acid of the invention within a cell, cell line or microorganism may be modified by inserting a DNA regulatory element heterologous to the endogenous gene of interest into the genome of a cell, stable cell line or cloned microorganism such that the inserted regulatory element is operatively linked with an endogenous gene and controls, modulates or activates the endogenous gene. For example, endogenous genes of the invention which are normally “transcriptionally silent”, i.e., genes which are normally not expressed, or are expressed only at very low levels in a cell line or microorganism, may be activated by inserting a regulatory element which is capable of promoting the expression of a normally expressed gene product in that cell line or microorganism. Alternatively, transcriptionally silent, endogenous genes of the invention may be activated by insertion of a promiscuous regulatory element that works across cell types.

A heterologous regulatory element may be inserted into a stable cell line or cloned microorganism, such that it is operatively linked with and activates expression of an endogenous gene corresponding to a nucleic acid of the invention, using techniques, such as targeted homologous recombination, which are well known to those of skill in the art (See, e.g., U.S. Pat. Nos. 5,272,071 and 5,968,502; International Publication Nos. WO 91/06667 and WO 94/12650). Alternatively, non-targeted techniques (e.g., non-homologous recombination) well known in the art can be used (see, e.g., International Publication No. WO 99/15650).

Another aspect of the invention pertains to host cells into which a recombinant expression vector of the invention has been introduced. The terms “host cell” and “recombinant host cell” are used interchangeably herein. It is understood that such terms refer not only to the particular subject cell but to the progeny or potential progeny of such a cell. Because certain modifications may occur in succeeding generations due to either mutation or environmental influences, such progeny may not, in fact, be identical to the parent cell, but are still included within the scope of the term as used herein.

Accordingly, the present invention provides a host cell having an expression vector comprising a nucleic acid of the invention, or a variant thereof. A host cell can be any prokaryotic (e.g., E. coli) or eukaryotic cell (e.g., insect cells, yeast or mammalian cells). The invention also provides a method for expressing a nucleic acid of the invention thus making the encoded polypeptide (e.g., clk-2) comprising the steps of (a) culturing a cell comprising a recombinant nucleic acid of the invention under conditions that allow said polypeptide to be expressed by said cell; and isolating the expressed polypeptide.

Vector DNA can be introduced into prokaryotic or eukaryotic cells via conventional transformation or transfection techniques. As used herein, the terms “transformation” and “transfection” are intended to refer to a variety of art-recognized techniques for introducing foreign nucleic acid into a host cell, including calcium phosphate or calcium chloride co-precipitation, DEAE-dextran-mediated transfection, lipofection, or electroporation. Suitable methods for transforming or transfecting host cells can be found in Sambrook, et al. (supra), and other laboratory manuals.

For stable transfection of mammalian cells, it is known that, depending upon the expression vector and transfection technique used, only a small fraction of cells may integrate the foreign DNA into their genome. In order to identify and select these integrants, a gene that encodes a selectable marker (e.g., for resistance to antibiotics) is generally introduced into the host cells along with the gene of interest. Preferred selectable markers include those which confer resistance to drugs, such as G4 18, hygromycin and methotrexate. Cells stably transfected with the introduced nucleic acid can be identified by drug selection (e.g., cells that have incorporated the selectable marker gene will survive, while the other cells die).

A host cell of the invention, such as a prokaryotic or eukaryotic host cell in culture, can be used to produce a polypeptide of the invention. Accordingly, the invention further provides methods for producing a polypeptide of the invention using the host cells of the invention. In one embodiment, the method comprises culturing the host cell of invention (into which a recombinant expression vector encoding a polypeptide of the invention has been introduced) in a suitable medium such that the polypeptide is produced. In another embodiment, the method further comprises isolating the polypeptide of the invention from the medium or the host cell.

5.3.1 Reporter Genes

The invention provides for reporter genes to ascertain the effects of test compounds on expression levels of nucleic acid and polypeptide molecules of the invention (e.g., clk-2) in order to aid in the identification of agents of the invention. In general, changes in the amount of a reporter gene product is indicative of the effect of the test compound on the expression level of the nucleic acid and polypeptide molecules of the invention. In another embodiment, the reporter gene is used to ascertain the effects of test compounds on expression patterns of polypeptide molecules of the invention (e.g., clk-2). In general, changes in the localization (e.g., intracellular localization) of a reporter gene product is indicative of the effect of the test compound on the expression pattern of the polypeptide molecules of the invention. In another embodiment, reporter genes under the control of upstream regulatory sequences of nucleic acids of the invention can be used in the production of transgenic animals (see Section 5.4).

Reporter genes include, but are not limited to, luciferase, green fluorescent protein, beta-galactosidase, chloramphenicol acetyltransferase, and alkaline phosphatase. Such methods are well known to one of skill in the art. In a preferred embodiment, the reporter gene is easily assayed and has an activity which is not normally found in the host cell.

In one embodiment, luciferase is the reporter gene. Luciferases are enzymes that emit light in the presence of oxygen and a substrate (luciferin) and which have been used for real-time, low-light imaging of gene expression in cell cultures, individual cells, whole organisms, and transgenic organisms (reviewed by Greer & Szalay, 2002, Luminescence 17:43-74).

As used herein, the term “luciferase” is intended to embrace all luciferases, or recombinant enzymes derived from luciferases which have luciferase activity. The luciferase genes from fireflies have been well characterized, for example, from the Photinus and Luciola species (see, e.g., International Patent Publication No. WO 95/25798 for Photinus pyralis, European Patent Application No. EP 0 524 448 for Luciola cruciata and Luciola lateralis, and Devine et al., 1993, Biochim. Biophys. Acta 1173:121-132 for Luciola mingrelica). Other eucaryotic luciferase genes include, but are not limited to, the sea panzy (Renilla reniformis, see, e.g., Lorenz et al., 1991, PNAS 88:4438-4442), and the glow worm (Lampyris noctiluca, see e.g., Sula-Newby et al., 1996, Biochem J. 313:761-767). Bacterial luciferin-luciferase systems include, but are not limited to, the bacterial lux genes of terrestrial Photorhabdus luminescens (see, e.g., Manukhov et al., 2000, Genetika 36:322-30) and marine bacteria Vibrio fischeri and Vibrio harveyi (see, e.g., Miyamoto et al., 1988, J. Biol. Chem. 263:13393-9, and Cohn et al., 1983, PNAS 80:120-3, respectively). The luciferases encompassed by the present invention also includes the mutant luciferases described in U.S. Pat. No. 6,265,177.

In another embodiment, green fluorescent protein (“GFP”) is the reporter gene. GFP is a 238 amino acid protein with amino acids65 to 67 involved in the formation of the chromophore which does not require additional substrates or cofactors to fluoresce (see, e.g., Prasher et al., 1992, Gene 111:229-233; Yang et al., 1996, Nature Biotechnol. 14:1252-1256; and Cody et al., 1993, Biochemistry 32:1212-1218).

As used herein, the term “green fluorescent protein” or “GFP” is intended to embrace all GFPs (including the various forms of GFPs which exhibit colors other than green), or recombinant enzymes derived from GFPs which have GFP activity. The native gene for GFP was cloned from the bioluminescent jellyfish Aequorea victoria (see, e.g., Morin et al., 1972, J. Cell Physiol. 77:313-318). Wild type GFP has a major excitation peak at 395 nm and a minor excitation peak at 470 nm. The absorption peak at 470 nm allows the monitoring of GFP levels using standard fluorescein isothiocyanate (FITC) filter sets. Mutants of the GFP gene have been found useful to enhance expression and to modify excitation and fluorescence. For example, mutant GFPs with alanine, glycine, isoleucine, or threonine substituted for serine at position 65 result in mutant GFPs with shifts in excitation maxima and greater fluorescence than wild type protein when excited at 488 m (see, e.g., Heim et al., 1995, Nature 373:663-664; U.S. Pat. No. 5,625,048; Delagrave et al., 1995, Biotechnology 13:151-154; Cormack et al., 1996, Gene 173:33-38; and Cramer et al., 1996, Nature Biotechnol. 14:315-319). The ability to excite GFP at 488 nm permits the use of GFP with standard fluorescence activated cell sorting (“FACS”) equipment. In another embodiment, GFPs are isolated from organisms other than the jellyfish, such as, but not limited to, the sea pansy, Renilla reriformis.

Techniques for labeling cells with GFP in general are described in U.S. Pat. Nos. 5,491,084 and 5,804,387; Chalfie et al., 1994, Science 263:802-805; Heim et al., 1994, PNAS 91:12501-12504; Morise et al., 1974, Biochemistry 13:2656-2662; Ward et al., 1980, Pholochem. Photobiol. 31:611-615; Rizzuto et al., 1995, Curr. Biology 5:635-642; and Kaether & Gerdes, 1995, FEBS Lett. 369:267-271. The expression of GFPs in E. coli and C. elegans is described in U.S. Pat. No. 6,251,384. The expression of GFP in plant cells is discussed in Hu & Cheng, 1995, FEBS Lett. 369:331-33, and GFP expression in Drosophila is described in Davis et al., 1995, Dev. Biology 170:726-729.

In another embodiment, beta galactosidase (“β-gal”) is used as a reporter gene. β-gal is an enzyme that catalyzes the hydrolysis of β-galactosides (e.g., lactose) as well as galactoside analogs (e.g., o-nitrophenyl-β-D-galactopyranoside (“ONPG”) and chlorophenol red-β-D-galactopyranoside (“CPRG”)) (see, e.g., Nielsen et al., 1983 PNAS 80:5198-5202; Eustice et al., 1991, Biotechniques 11:739-742; and Henderson et al., 1986, Clin. Chem. 32:1637-1641).

As used herein, the term “beta galactosidase” or “β-gal” is intended to embrace all β-gals, including lacZ gene products, or recombinant enzymes derived from β-gals which have β-gal activity. The β-gal gene functions well as a reporter gene because the protein product is extremely stable, resistant to proteolytic degradation in cellular lysates, and easily assayed. In an embodiment where ONPG is the substrate, β-gal activity can be quantitated with a spectrophotometer or microplate reader to determine the amount of ONPG converted at 420 nm. In an embodiment when CPRG is the substrate, β-gal activity can be quantitated with a spectrophotometer or microplate reader to determine the amount of CPRG converted at 570 to 595 nm. In yet another embodiment, the β-gal activity can be visually ascertained by plating bacterial cells transformed with a β-gal construct onto plates containing Xgal and IPTG. Bacterial colonies that are dark blue indicate the presence of high β-gal activity and colonies that are varying shades of blue indicate varying levels of β-gal activity.

In one embodiment, chloramphenicol acetyltransferase (“CAT”) is used as a reporter gene. CAT is commonly used as a reporter gene in mammalian cell systems because mammalian cells do not have detectable levels of CAT activity. The assay for CAT involves incubating cellular extracts with radiolabeled chloramphenicol and appropriate co-factors, separating the starting materials from the product by, for example, thin layer chromatography (“TLC”), followed by scintillation counting (see, e.g., U.S. Pat. No. 5,726,041).

As used herein, the term “chloramphenicol acetyltransferase” or “CAT” is intended to embrace all CATs, or recombinant enzymes derived from CAT which have CAT activity. While it is preferable that a reporter system which does not require cell processing, radioisotopes, and chromatographic separations would be more amenable to high through-put screening, CAT as a reporter gene may be preferable in situations when stability of the reporter gene is important. For example, the CAT reporter protein has an in vivo half life of about 50 hours, which is advantageous when an accumulative versus a dynamic change type of result is desired.

In another embodiment, secreted alkaline phosphatase (“SEAP”) is used as a reporter gene. SEAP enzyme is a truncated form of alkaline phosphatase, in which the cleavage of the transmembrane domain of the protein allows it to be secreted from the cells into the surrounding media. In a preferred embodiment, the alkaline phosphatase is isolated from human placenta.

As used herein, the term “secreted alkaline phosphatase” or “SEAP” is intended to embrace all SEAP or recombinant enzymes derived from SEAP which have alkaline phosphatase activity. SEAP activity can be detected by a variety of methods including, but not limited to, measurement of catalysis of a fluorescent substrate, immunoprecipitation, HPLC, and radiometric detection. The luminescent method is preferred due to its increased sensitivity over calorimetric detection methods. The advantages of using SEAP is that a cell lysis step is not required since the SEAP protein is secreted out of the cell, which facilitates the automation of sampling and assay procedures. A cell-based assay using SEAP for use in cell-based assessment of inhibitors of the Hepatitis C virus protease is described in U.S. Pat. No. 6,280,940.

5.4 Transgenic Animals

The invention provides for transgenic animals to be used in the methods of the invention. Such transgenic animals are used in 1) the identification and characterization of signaling pathways in which polypeptides of the invention participate; 2) identification and characterization of phenotypes associated with the mutation or abnormal expression of the polypeptides of the invention; and 3) for use in screening to identify agents of the invention which modulate expression/function of polypeptides of the invention.

As used herein, a “transgenic animal” is a non-human animal, preferably a C. elegans nematode or a mammal, more preferably a rodent such as a rat or mouse, in which one or more of the cells of the animal either includes a transgene or contains a deletion in an endogenous gene. The transgene may or may not be integrated into the transgenic animals's genome. In embodiments where the transgene is integrated, progeny of the transgenic animal inherited the transgene. In one embodiment, a transgene comprises a regulatory sequence and is integrated such that the transgenic regulatory sequence controls expression of the animal's endogenous gene. In another embodiment, a transgene comprises a regulatory sequence operably associated with a nucleic acid sequence to be expressed. Such a transgenic nucleic acid may encode a protein or may be an antisense molecule. A transgenic animal contains either an exogenous DNA (i.e., transgene which may or may not be under the control of an inducible or tissue specific promoter and may or may not be derived from the same species as the host animal) which directs the expression of a heterologous polypeptide or a disruption in the DNA of an endogenous gene which remains in the genome of the mature animal. Such additions or deletions of genomic material may or may not be accomplished through the use of homologous recombination. Methods of producing such animal models using novel genes and proteins are well known in the art (see e.g., PCT International Publication No. WO 96/34099). Such models include but are not limited to the following three embodiments.

In a first embodiment, animals are provided in which a normal gene of the invention has been recombinantly introduced into the genome of the animal as an additional gene, under the regulation of either an exogenous or an endogenous promoter element, and as either a minigene or a large genomic fragment. Animals are also provided in which a normal gene has been recombinantly substituted for one or both copies of the animal's homologous gene by homologous recombination or gene targeting.

In a second embodiment, animals are provided in which a mutant gene of the invention has been recombinantly introduced into the genome of the animal as an additional gene, under the regulation of either an exogenous or an endogenous promoter element, and as either a minigene or a large genomic fragment. Animals are also provided in which a mutant gene has been recombinantly substituted for one or both copies of the animal's homologous gene by homologous recombination or gene targeting.

In a third embodiment, animals are provided in which a mutant version of one of that animal's own genes (bearing, for example, a specific mutation corresponding to, or similar to, a pathogenic mutation of a polypeptide molecule of the invention from another species) has been recombinantly introduced into the genome of the animal as an additional gene, under the regulation of either an exogenous or an endogenous promoter element, and as either a minigene or a large genomic fragment.

Mammals

DNA constructs will comprise at least a portion of the nucleic acid molecule of the invention (e.g., clk-2 gene sequence) and may or may not have a genetic modification/mutation. In one embodiment, a deletion of an endogenous gene is desired. In such an embodiment, the DNA construct will include regions of homology to the target locus such that the DNA construct will homologously recombine at the desired site and be inserted into the genome thus disrupting a particular endogenous gene. In this embodiment, at least 5, 10, 15, 25, 50, 100, 150, 200 consecutive nucleotides of the endogenous gene are disrupted. Methods for constructing homologous recombination vectors and homologous recombinant animals are described further in Bradley (1991, Curr. Opin. BioTechnology 2:823-829) and in International Publication Nos. WO 90/11354, WO 91/01140, WO 92/0968 and WO 93/04169. In another embodiment, expression of a transgene is desired. In such an embodiment, the DNA construct will include at least a portion of the reading frame of a polypeptide of the invention. Conveniently, markers for positive and negative selection are included. Methods for generating transgenic animals via embryo manipulation and microinjection, particularly animals such as mice, have become conventional in the art (see, e.g., U.S. Pat. Nos. 4,736,866; 4,870,009; 4,873,191; Hogan, 1986, Manipulating the Mouse Embryo, Cold Spring Harbor Laboratory Press, Cold Spring Harbor, N.Y.; Wakayama et al., 1999, PNAS 96:14984-14989).

For embryonic stem (ES) cells, an ES cell line may be employed, or embryonic cells may be obtained freshly from a host, e.g. mouse, rat, guinea pig, etc. Such cells are grown on an appropriate fibroblast-feeder layer or grown in the presence of leukemia inhibiting factor (LIF). When ES or embryonic cells have been transformed, they may be used to produce transgenic animals. After transformation, the cells are plated onto a feeder layer in an appropriate medium. Cells containing the DNA construct may be detected by employing a selective medium. After sufficient time for colonies to grow, they are picked and analyzed for the occurrence of homologous recombination or integration of the construct. Those colonies that are positive may then be used for embryo manipulation and blastocyst injection. Blastocysts are obtained from 4 to 6 week old superovulated females. The ES cells are trypsinized, and the modified cells are injected into the blastocoel of the blastocyst. After injection, the blastocysts are returned to each uterine horn of pseudopregnant females. Females are then allowed to go to term and the resulting offspring screened for the construct. By providing for a different phenotype of the blastocyst and the genetically modified cells, chimeric progeny can be readily detected.

The chimeric animals are screened for the presence of the modified gene and males and females having the modification are mated to produce homozygous progeny. If the gene alterations cause lethality at some point in development, tissues or organs can be maintained as allogeneic or congenic grafts or transplants, or in in vitro culture. The transgenic animals may be any non-human mammal, such as laboratory animals, domestic animals, etc. The transgenic animals or cell therefrom may be used in functional studies, drug screening, etc.

C. elegans

Transgenic C. elegans can be made by any method known in the art. See, for example, Mello and Fire, 1995, DNA Transformation in Methods in Cell Biology: Caenorhabditis elegans: Modern biological analysis of an organism in volume 48 (ed. H.F. Epstein and D.C. Shakes) New York: Academic Press pp.452-82 and C. elegans: A Practical Approach by Hope, Oxford University Press, England 1999.

5.5 Antibodies

The present invention encompasses antibodies, or fragments thereof that immunospecifically bind to a polypeptide molecule of the invention (e.g., clk-2). The antibodies of the invention can be polyclonal antibodies or monoclonal antibodies. The term “immunospecifically” as used herein refers to the ability of an antibody of the invention to bind a clk-2 polypeptide without cross-reactivity with other non-clk-2 polypeptides.

In one embodiment, the invention provides uses of substantially purified antibodies or fragments thereof, including human, non-human, or humanized antibodies or fragments thereof, which antibodies or fragments immunospecifically bind to a polypeptide of the invention comprising an amino acid sequence selected from the group consisting of: the amino acid sequence of SEQ ID NOs:2, 3, 8, 9, 10, 11, 12, 13, 14, 17, 18, 19, 25, 26, 27, 28, 29, 30, 31, 32; and an amino acid sequence which is encoded by the polynucleotide consisting of SEQ ID NOs:1, 4, 5, 6, 7, 15, 16, 20, 21, 22, 23, 24; or a fragment of at least 8 contiguous amino acid residues of the amino acid sequence of SEQ ID NOs:2, 3, 8, 9, 10, 11, 12, 13, 14, 17, 18, 19, 25, 26, 27, 28, 29, 30, 31, 32. Non-human antibodies can be goat, mouse, sheep, horse, chicken, rabbit, or rat antibodies.

In other embodiments, the invention provides substantially purified antibodies or fragments thereof, including human, non-human, or humanized antibodies or fragments thereof, which antibodies or fragments immunospecifically bind to a polypeptide of the invention comprising: i) a naturally occurring allelic variant of a polypeptide comprising the amino acid sequence of any of SEQ ID NOs:2, 3, 8, 9, 10, 11, 12, 13, 14, 17, 18, 19, 25, 26, 27, 28, 29, 30, 31, 32, wherein the polypeptide is encoded by a nucleic acid molecule which hybridizes with a nucleic acid molecule consisting of the nucleotide sequence of any of SEQ ID NOs: 1, 4, 5, 6, 7, 15, 16, 20, 21, 22, 23, 24, or a complement thereof under stringent conditions; ii) a polypeptide that is encoded by a nucleic acid molecule comprising a nucleotide sequence which is at least 90% identical to a nucleic acid consisting of the nucleotide sequence of any of SEQ ID NOs: 1, 4, 5, 6, 7, 15, 16, 20, 21, 22, 23, 24 or a complement thereof, and iii) a polypeptide that is at least 90% identical to the amino acid sequence of any of SEQ ID NOs:2, 3, 8, 9, 10, 11, 12, 13, 14, 17, 18, 19, 25, 26, 27, 28, 29, 30, 31, 32. In one embodiment, the antibody of the invention immunospecifically binds a clk-2 polypeptide but not a polypeptide consisting of any of SEQ IDNOs:2, 3, 8, 9, 10, 11, 12, 13, 14, 17, 18, 19, 25, 26, 27, 28, 29, 30, 31, 32. Non-human antibodies can be goat, mouse, sheep, horse, chicken, rabbit, or rat antibodies. The antibodies of the invention can be used in the methods of the invention.

In specific embodiments, the antibody binds to a domain of a polypeptide molecule of the invention, and prevents binding of the polypeptide to an endogenous binding partner, or causes the polypeptide to be degraded. In specific embodiments, the antibody binds to derivatives of a polypeptide of the invention, such as but not limited to polypeptides in which one or more of the serine, threonine and tyrosine residues of the polypeptides are phosphorylated. In certain embodiments, the antibody can only bind the polypeptide when one or more of the serine, threonine and tyrosine residue(s) are phosphorylated. Yet in other embodiments, the antibody can only bind completely unphosphorylated polypeptide or polypeptide that are partially unphosphorylated at specific sites.

In various embodiments, the antibodies of the present invention bind to the same epitope as any the antibodies that immunospecifically bind to polypeptides of the invention or competes with any of the antibodies that immunospecifically bind to polypeptides of the invention, e.g. as assayed by ELISA or any other appropriate immunoassay. As used herein, the term “epitope” refers to a portion of a polypeptide of the invention having antigenic or immunogenic activity in an animal, preferably in a mammal, and most preferably in a human. An epitope having immunogenic activity is a portion of a polypeptide of the invention that elicits an antibody response in an animal. An epitope having antigenic activity is a portion of a polypeptide of the invention to which an antibody immunospecifically binds as determined by any method well known in the art, for example, by immunoassays. Antigenic epitopes need not necessarily be immunogenic. An epitope can comprise post-translationally modified residues on the polypeptide, e.g., glycosylations and phosphorylations.

As used herein, the term “antibodies or fragments thereof that immunospecifically bind to a polypeptide of the invention” refers to antibodies or fragments thereof that specifically bind to a polypeptide of the invention (e.g., clk-2) and do not specifically bind to other polypeptides. Preferably, antibodies or fragments that immunospecifically bind to a polypeptide of the invention or a fragment thereof do not cross-react with other antigens. Antibodies or fragments that immunospecifically bind to a polypeptide of the invention can be identified, for example, by immunoassays or other techniques known to those of skill in the art. Antibodies of the invention include, but are not limited to, synthetic antibodies, monoclonal antibodies, recombinantly produced antibodies, multispecific antibodies, human antibodies, humanized antibodies, chimeric antibodies, single-chain Fvs (sFv), single chain antibodies, intrabodies, Fab fragments, F(ab′) fragments, disulfide-linked Fvs (sdFv), and anti-idiotypic (anti-Id) antibodies, and epitope-binding fragments of any of the above. In particular, antibodies of the present invention include immunoglobulin molecules and immunologically active portions of immunoglobulin molecules, i.e., molecules that contain an antigen binding site that immunospecifically binds to an antigen of a polypeptide of the invention (e.g., one or more complementarity determining regions (CDRs) of an antibody). The immunoglobulin molecules of the invention can be of any type (e.g., IgG, IgE, IgM, IgD, IgA and IgY), class (e.g., IgG₁, IgG₂, IgG₃, IgG₄, IgA, and IgA₂) or subclass of immunoglobulin molecule.

In various embodiments, the antibodies of the invention, or fragments thereof, can be chimeric and/or humanized antibodies. The antibodies used in the methods of the invention may be from any animal origin including birds and mammals (e.g., human, murine, donkey, sheep, rabbit, goat, guinea pig, camel, horse, or chicken). Preferably, the antibodies are human or humanized monoclonal antibodies. As used herein, “human” antibodies include antibodies having the amino acid sequence of a human immunoglobulin and include antibodies isolated from human immunoglobulin libraries or from mice that express antibodies from human genes.

The antibodies used in the methods of the present invention may be monospecific, bispecific, trispecific or of greater multi specificity. Multi specific antibodies may immunospecifically bind to different epitopes of a polypeptide molecule of the invention or may immunospecifically bind to a polypeptide molecule of the invention as well a heterologous epitope, such as a heterologous polypeptide as described by Segal in U.S. Pat. No. 4,676,980. See, e.g., International Publication Nos. WO 93/17715, WO 92/08802, WO 91/00360, and WO 92/05793; Tutt, et al., 1991, J. Immunol. 147:60-69; U.S. Pat. Nos. 4,474,893, 4,714,681, 4,925,648, 5,573,920, and 5,601,819; and Kostelny et al., 1992, J. Immunol. 148:1547-1553.

The antibodies used in the methods of the invention include derivatives that are modified, i.e, by the covalent attachment of any type of molecule to the antibody such that covalent attachment. For example, but not by way of limitation, the antibody derivatives include antibodies that have been modified, e.g., by glycosylation, acetylation, pegylation, phosphorylation, amidation, derivatization by known protecting/blocking groups, proteolytic cleavage, linkage to a cellular ligand or other protein, etc. Any of numerous chemical modifications may be carried out by known techniques, including, but not limited to, specific chemical cleavage, acetylation, formylation, metabolic synthesis of tunicamycin, etc. Additionally, the derivative may contain one or more non-classical amino acids.

The present invention also provides antibodies of the invention or fragments thereof that comprise a framework region known to those of skill in the art. Preferably, the antibody of the invention or fragment thereof is human or humanized. In a specific embodiment, the antibody of the invention or fragment thereof comprises one or more CDRs from any antibody that immunospecifically binds a polypeptide molecule of the invention. In a more specific embodiment, the antibody of the invention or fragment thereof comprises one or more CDRs from any antibody that immunospecifically recognizes a polypeptide molecule of the invention.

The present invention encompasses single domain antibodies, including camelized single domain antibodies (see e.g., Muyldermans et al., 2001, Trends Biochem. Sci. 26:230; Nuttall et al., 2000, Cur. Pharm. Biotech. 1:253; Reichmann and Muyldermans, 1999, J. Immunol. Meth. 231:25; International Publication Nos. WO 94/04678 and WO 94/25591; U.S. Pat. No. 6,005,079). In one embodiment, the present invention provides single domain antibodies comprising two V_{H} domains having modifications such that single domain antibodies are formed and having the amino acid sequence of any of the V_{H} domains from any antibody that immunospecifically binds a polypeptide molecule of the invention. In another embodiment, the present invention also provides single domain antibodies comprising two V_{H} domains comprising one or more of the V_{H} CDRs from any antibody that immunospecifically binds a polypeptide molecule of the invention.

The methods of the present invention also encompass the use of antibodies or fragments thereof that have half-lives (e.g., serum half-lives) in a mammal, preferably a human, of greater than 15 days, preferably greater than 20 days, greater than 25 days, greater than 30 days, greater than 35 days, greater than 40 days, greater than 45 days, greater than 2 months, greater than 3 months, greater than 4 months, or greater than 5 months. The increased half-lives of the antibodies of the present invention or fragments thereof in a mammal, preferably a human, results in a higher serum titer of said antibodies or antibody fragments in the mammal, and thus, reduces the frequency of the administration of said antibodies or antibody fragments and/or reduces the concentration of said antibodies or antibody fragments to be administered. Antibodies or fragments thereof having increased in vivo half-lives can be generated by techniques known to those of skill in the art. For example, antibodies or fragments thereof with increased in vivo half-lives can be generated by modifying (e.g., substituting, deleting or adding) amino acid residues identified as involved in the interaction between the Fc domain and the FcRn receptor (see, e.g., International Publication No. WO 97/34631). Antibodies or fragments thereof with increased in vivo half-lives can be generated by attaching to said antibodies or antibody fragments polymer molecules such as high molecular weight polyethyleneglycol (PEG). PEG can be attached to said antibodies or antibody fragments with or without a multifunctional linker either through site-specific conjugation of the PEG to the—or C-terminus of said antibodies or antibody fragments or via epsilon-amino groups present on lysine residues. Linear or branched polymer derivatization that results in minimal loss of biological activity will be used. The degree of conjugation will be closely monitored by SDS-PAGE and mass spectrometry to ensure proper conjugation of PEG molecules to the antibodies. Unreacted PEG can be separated from antibody-PEG conjugates by, e.g., size exclusion or ion-exchange chromatography.

The present invention also encompasses the use of antibodies or antibody fragments comprising the amino acid sequence of an antibody that immunospecifically binds a polypeptide molecule of the invention with mutations (e.g., one or more amino acid substitutions) in the framework or variable regions. Preferably, mutations in these antibodies maintain or enhance the avidity and/or affinity of the antibodies for the particular antigen(s) to which they immunospecifically bind. Standard techniques known to those skilled in the art (e.g., immunoassays) can be used to assay the affinity of an antibody for a particular antigen.

Standard techniques known to those skilled in the art can be used to introduce mutations in the nucleotide sequence encoding an antibody, or fragment thereof, including, e.g., site-directed mutagenesis and PCR-mediated mutagenesis, which results in amino acid substitutions. Preferably, the derivatives include less than 15 amino acid substitutions, less than 10 amino acid substitutions, less than 5 amino acid substitutions, less than 4 amino acid substitutions, less than 3 amino acid substitutions, or less than 2 amino acid substitutions relative to the original antibody or fragment thereof. In a preferred embodiment, the derivatives have conservative amino acid substitutions made at one or more predicted non-essential amino acid residues.

5.5.1 Methods of Producing Antibodies

The antibodies or fragments thereof can be produced by any method known in the art for the synthesis of antibodies, in particular, by chemical synthesis or preferably, by recombinant expression techniques.

Monoclonal antibodies can be prepared using a wide variety of techniques known in the art including the use of hybridoma, recombinant, and phage display technologies, or a combination thereof. For example, monoclonal antibodies can be produced using hybridoma techniques including those known in the art and taught, for example, in Harlow et al., Antibodies. A Laboratory Manual, (Cold Spring Harbor Laboratory Press, 2nd ed. 1988); Hammerling, et al., in: Monoclonal Antibodies and T-Cell Hybridomas 563-681 (Elsevier, N.Y., 1981). The term “monoclonal antibody” as used herein is not limited to antibodies produced through hybridoma technology. The term “monoclonal antibody” refers to an antibody that is derived from a single clone, including any eukaryotic, prokaryotic, or phage clone, and not the method by which it is produced.

Methods for producing and screening for specific antibodies using hybridoma technology are routine and well known in the art. Briefly, mice can be immunized with a polypeptide molecule of the invention (either the full length protein or a domain thereof) and once an immune response is detected, e.g., antibodies specific for the polypeptide molecule of the invention are detected in the mouse serum, the mouse spleen is harvested and splenocytes isolated. The splenocytes are then fused by well known techniques to any suitable myeloma cells, for example cells from cell line SP20 available from the ATCC. Hybridomas are selected and cloned by limited dilution. Additionally, a RIMMS (repetitive immunization, multiple sites) technique can be used to immunize an animal (Kilpatrick et al., 1997, Hybridoma 16:381-9). Briefly, partially purified antigen is emulsified in adjuvant and injected into 12 subcutaneous sites proximal to draining lymph nodes at days 0, 3, 6, 8, and 10. Seventy two hours after the final series of antigen injections, lymphocytes are harvested from the lymph nodes and somatically fused (using PEG) with P3XBcl-2-13 cells.

Hybridoma clones are assayed by methods known in the art for cells that secrete antibodies capable of binding a polypeptide of the invention. Ascites fluid, which generally contains high levels of antibodies, can be generated by immunizing mice with positive hybridoma clones.

Human antibodies can also be produced using transgenic mice which are incapable of expressing functional endogenous immunoglobulins, but which can express human immunoglobulin genes. For example, the human heavy and light chain immunoglobulin gene complexes may be introduced randomly or by homologous recombination into mouse embryonic stem cells. For a detailed discussion of this technology for producing human antibodies and human monoclonal antibodies and protocols for producing such antibodies, see, e.g., International Publication Nos. WO 98/24893, WO 96/34096, and WO 96/33735; and U.S. Pat. Nos. 5,413,923, 5,625,126, 5,633,425, 5,569,825, 5,661,016, 5,545,806, 5,814,318, and 5,939,598. In addition, companies such as Abgenix, Inc. (Freemont, Calif.), Genpharm (San Jose, Calif.) and Medarex (Princeton, N.J.) can be engaged to provide human antibodies directed against a selected antigen using technology similar to that described above.

5.5.2 Polynucleotides Encoding an Antibody

The methods of the invention also encompass polynucleotides that encode or hybridize under high stringency, intermediate or lower stringency hybridization conditions to polynucleotides that encode an antibody of the invention.

The polynucleotides may be obtained, and the nucleotide sequence of the polynucleotides determined, by any method known in the art. Since the amino acid sequences of the antibodies are known, nucleotide sequences encoding these antibodies can be determined using methods well known in the art, i.e., nucleotide codons known to encode particular amino acids are assembled in such a way to generate a nucleic acid that encodes the antibody or fragment thereof of the invention. Such a polynucleotide encoding the antibody may be assembled from chemically synthesized oligonucleotides (e.g., as described in Kutmeier et al., 1994, BioTechniques 17:242), which, briefly, involves the synthesis of overlapping oligonucleotides containing portions of the sequence encoding the antibody, annealing and ligating of those oligonucleotides, and then amplification of the ligated oligonucleotides by PCR.

Alternatively, a polynucleotide encoding an antibody may be generated from nucleic acid from a suitable source. If a clone containing a nucleic acid encoding a particular antibody is not available, but the sequence of the antibody molecule is known, a nucleic acid encoding the immunoglobulin may be chemically synthesized or obtained from a suitable source (e.g., an antibody cDNA library, or a cDNA library generated from, or nucleic acid, preferably poly A+ RNA, isolated from, any tissue or cells expressing the antibody, such as hybridoma cells selected to express an antibody of the invention) by PCR amplification using synthetic primers hybridizable to the 3′ and 5 ′ ends of the sequence or by cloning using an oligonucleotide probe specific for the particular gene sequence to identify, e.g., a cDNA clone from a cDNA library that encodes the antibody. Amplified nucleic acids generated by PCR may then be cloned into replicable cloning vectors using any method well known in the art.

Once the nucleotide sequence of the antibody is determined, the nucleotide sequence of the antibody may be manipulated using methods well known in the art for the manipulation of nucleotide sequences, e.g., recombinant DNA techniques, site directed mutagenesis, PCR, etc. (see, for example, the techniques described in Sambrook et al., supra and Ausubel et al., eds., 1998, Current Protocols in Molecular Biology, John Wiley & Sons, NY), to generate antibodies having a different amino acid sequence, for example to create amino acid substitutions, deletions, and/or insertions. Other alterations to the polynucleotide are encompassed by the present invention and within the skill of the art.

5.5.3 Intrabodies

In certain embodiments, because of the intracellular location of the polypeptides of the invention, it may be advantageous for an antibody to bind the antigen intracellularly i.e., an intrabody. An intrabody comprises at least a portion of an antibody that is capable of immunospecifically binding an antigen and preferably does not contain sequences coding for its secretion. Such an intrabody can be used to modulate the activity of the polypeptide of the invention to which it binds. In one embodiment, an antagonistic intrabody is administered to decrease the activity of a polypeptide of the invention. In another embodiment, an agonisitc intrabody is administered to increase the activity of a polypeptide of the invention. In another embodiment, an intrabody of the invention is administered such that it localizes to a specific subcellular compartment and thus modulates a polypeptide of the invention exclusively in that location.

In one embodiment, the intrabody comprises a single-chain Fv (“sFv”). sFvs are antibody fragments comprising the V_{H} and V_{L} domains of antibody, wherein these domains are present in a single polypeptide chain. Generally, the sFv polypeptide further comprises a polypeptide linker between the V_{H} and V_{L} domains which enables the sFv to form the desired structure for antigen binding. For a review of sFvs see Pluckthun in The Pharmacology of Monoclonal Antibodies, vol. 113, Rosenburg and Moore eds. Springer-Verlag, New York, pp. 269-315 (1994). In a further embodiment, the intrabody preferably does not encode an operable secretory sequence and thus remains within the cell (see generally Marasco, W A, 1998, “Intrabodies: Basic Research and Clinical Gene Therapy Applications” Springer:New York).

Generation of intrabodies is well-known to the skilled artisan and is described, for example, in U.S. Pat. Nos. 6,004,940; 6,072,036; 5,965,371. Further, the construction of intrabodies is discussed in Ohage and Steipe, 1999, J. Mol. Biol. 291:1119-1128; Ohage et al., 1999, J. Mol. Biol. 291:1129-1134; and Wirtz and Steipe, 1999, Protein Science 8:2245-2250. Recombinant molecular biological techniques such as those described for recombinant production of antibodies (e.g., Sections 5.5, 5.5.1, 5.5.2) may also be used in the generation of intrabodies.

In another embodiment, intrabodies of the invention retain at least about 75% of the binding effectiveness of the complete antibody (i.e., having the entire constant domain as well as the variable regions) to the antigen. More preferably, the intrabody retains at least 85% of the binding effectiveness of the complete antibody. Still more preferably, the intrabody retains at least 90% of the binding effectiveness of the complete antibody. Even more preferably, the intrabody retains at least 95% of the binding effectiveness of the complete antibody.

In producing intrabodies, polynucleotides encoding variable region for both the V_{H} and V_{L} chains of interest can be cloned by using, for example, hybridoma mRNA or splenic mRNA as a template for PCR amplification of such domains (Huse et al., 1989, Science 246:1276). In one preferred embodiment, the polynucleotides encoding the V_{H} and V_{L} domains are joined by a polynucleotide sequence encoding a linker to make a single chain antibody (sFv). The sFv typically comprises a single peptide with the sequence V_{H}-linker-V_{L} or V_{L}-linker-V_{H}. The linker is chosen to permit the heavy chain and light chain to bind together in their proper conformational orientation (see for example, Huston, et al., 1991, Methods in Enzym. 203:46-121). In a further embodiment, the linker can span the distance between its points of fusion to each of the variable domains (e.g., 3.5 nm) to minimize distortion of the native Fv conformation. In such an embodiment, the linker is a polypeptide of at least 5 amino acid residues, at least 10 amino acid residues, at least 15 amino acid residues, or greater. In a further embodiment, the linker should not cause a steric interference with the V_{H} and V_{L} domains of the combining site. In such an embodiment, the linker is 35 amino acids or less, 30 amino acids or less, or 25 amino acids or less. Thus, in a most preferred embodiment, the linker is between 15-25 amino acid residues in length. In a further embodiment, the linker is hydrophilic and sufficiently flexible such that the V_{H} and V_{L} domains can adopt the conformation necessary to detect antigen. Intrabodies can be generated with different linker sequences inserted between identical V_{H} and V_{L} domains. A linker with the appropriate properties for a particular pair of V_{H} and V_{L} domains can be determined empirically by assessing the degree of antigen binding for each.

In another embodiment, intrabodies are expressed in the cytoplasm. In other embodiments, the intrabodies are localized to various intracellular locations. In such embodiments, specific localization sequences can be attached to the intrabody polypeptide to direct the intrabody to a specific location. Intrabodies can be localized, for example, to the following intracellular locations: endoplasmic reticulum (Munro et al., 1987, Cell 48:899-907; Hangejorden et al., 1991, J. Biol. Chem. 266:6015); nucleus (Lanford et al., 1986, Cell 46:575; Stanton et al., 1986, PNAS 83:1772; Harlow et al., 1985, Mol. Cell Biol. 5:1605; Pap et al., 2002, Exp. Cell Res. 265:288-93); nucleolar region (Seomi et al., 1990, J. Virology 64:1803; Kubota et al., 1989, Biochem. Biophys. Res. Comm. 162:963; Siomi et al., 1998, Cell 55:197); endosomal compartment (Bakke et al., 1990, Cell 63:707-716); mitochondrial matrix (Pugsley, A. P., 1989, “Protein Targeting”, Academic Press, Inc.); Golgi apparatus (Tang et al., 1992, J. Bio. Chem. 267:10122-6); liposomes (Letourneur et al., 1992, Cell 69:1183); peroxisome (Pap et al., 2002, Exp. Cell Res. 265:288-93); trans Golgi network (Pap et al., 2002, Exp. Cell Res. 265:288-93); and plasma membrane (Marchildon et al., 1984, PNAS 81:7679-82; Henderson et al., 1987, PNAS 89:339-43; Rhee et al., 1987, J. Virol. 61:1045-53; Schultz et al., 1984, J. Virol. 133:431-7; Ootsuyama et al., 1985, Jpn. J Can. Res. 76:1132-5; Ratner et al., 1985, Nature 313:277-84).

V_{H} and V_{L} domains are made up of the immunoglobulin domains that generally have a conserved structural disulfide bond. In embodiments where the intrabodies are expressed in a reducing environment (e.g., the cytoplasm), such a structural feature cannot exist. Mutations can be made to the intrabody polypeptide sequence to compensate for the decreased stability of the immunoglobulin structure resulting from the absence of disulfide bond formation. In one embodiment, the V_{H} and/or V_{L} domains of the intrabodies contain one or more point mutations such that their expression is stabilized in reducing environments (see Steipe et al., 1994, J. Mol. Biol. 240:188-92; Wirtz and Steipe, 1999, Protein Science 8:2245-50; Ohage and Steipe, 1999, J. Mol. Biol. 291:1119-28; Ohage et al., 1999, J. Mol. Biol. 291:1129-34).

In another embodiment, the recombinantly expressed intrabody protein is administered to a patient. Such an intrabody polypeptide must be intracellular to mediate a prophylactic or therapeutic effect. In this embodiment of the invention, the intrabody polypeptide is associated with a “membrane permeable sequence”. Membrane permeable sequences are polypeptides capable of penetrating through the cell membrane from outside of the cell to the interior of the cell. When linked to another polypeptide, membrane permeable sequences can also direct the translocation of that polypeptide across the cell membrane as well.

In yet another embodiment, the membrane permeable sequence is the hydrophobic region of a signal peptide (see, e.g., Hawiger, 1999, Curr. Opin. Chem. Biol. 3:89-94; Hawiger, 1997, Curr. Opin. Immunol. 9:189-94; U.S. Pat. Nos. 5,807,746 and 6,043,339). The sequence of a membrane permeable sequence can be based on the hydrophobic region of any signal peptide. The signal peptides can be selected, e.g., from the SIGPEP database (see e.g., von Heijne, 1987, Prot. Seq. Data Anal. 1:41-2; von Heijne and Abrahmsen, 1989, FEBS Lett. 224:439-46). When a specific cell type is to be targeted for insertion of an intrabody polypeptide, the membrane permeable sequence is preferably based on a signal peptide endogenous to that cell type. In another embodiment, the membrane permeable sequence is a viral protein (e.g., Herpes Virus Protein VP22) or fragment thereof (see e.g., Phelan et al., 1998, Nat. Biotechnol. 16:440-3). A membrane permeable sequence with the appropriate properties for a particular intrabody and/or a particular target cell type can be determined empirically by assessing the ability of each membrane permeable sequence to direct the translocation of the intrabody across the cell membrane.

In other embodiments, the membrane permeable sequence can be a derivative. In this embodiment, the amino acid sequence of a membrane permeable sequence has been altered by the introduction of amino acid residue substitutions, deletions, additions, and/or modifications. For example, a derivative membrane permeable sequence polypeptide can translocate through the cell membrane more efficiently or be more resistant to proteolysis.

The membrane permeable sequence can be attached to the intrabody in a number of ways. In one embodiment, the membrane permeable sequence and the intrabody are expressed as a fusion protein. In another embodiment, the membrane permeable sequence polypeptide is attached to the intrabody polypeptide after each is separately expressed recombinantly (see e.g., Zhang et al., 1998, PNAS 95:9184-9).

In yet another embodiment, a polynucleotide encoding an intrabody is administered to a patient (e.g., as in gene therapy). In this embodiment, methods as described in Section 5.7.4 can be used to administer the polynucleotide of the invention.

5.6 Identification of Agents of the Invention

The invention provides methods of assaying and screening for agents that can alter expression and/or activity of a clk-2 nucleic acid or clk-2 polypeptide of the invention. Although not intending to be bound by a particular mechanism of action, an agent of the invention can alter activity of a clk-2 polypeptide by, e.g., enhancing or interfering with clk-2 interaction with an endogenous binding partner (e.g., a polypeptide, lipid, or nucleic acid that interacts with clk-2 in a wild type organism), enhancing/interfering with clk-2 enzymatic activity, enhancing/interfering with clk-2 phosphorylation, etc. The methods generally involve incubating agents with animals or cells that express a nucleic acid or polypeptide molecule of the invention (e.g., clk-2) and then assaying for an alteration in phenotype thereby identifying an agent of the invention. The invention also encompasses the use of biochemical assays to identify test compounds that bind to polypeptide molecules of the invention. Compounds found to bind to a polypeptide molecule of the invention can then be assayed in C. elegans- or cell-based assays to determine any phenotype-altering properties.

As used herein, the term “agent” refers to a molecule that has a desired biological effect. Agents include, but are not limited to, proteinaceous molecules, including, but not limited to, peptide, polypeptide, protein, post-translationally modified protein, antibodies etc.; or a large molecule, including, but not limited to, inorganic or organic compounds; or a small molecule (less than 500 daltons), including, but not limited to, inorganic or organic compounds; or a nucleic acid molecule, including, but not limited to, double-stranded DNA, single-stranded DNA, double-stranded RNA, single-stranded RNA, or triple helix nucleic acid molecules. Agents can be natural products derived from any known organism (including, but not limited to, animals, plants, bacteria, fungi, protista, or viruses) or from a library of synthetic molecules. As used herein, the terms “agent” and “compound” are used interchangeably.

5.6.1 Biochemical Assays

5.6.1.1 In Vitro Binding Assays

Compounds that bind to a polypeptide of the invention (e.g., clk-2) can be identified by any method known in the art. For example, any method that detects an altered physical property (e.g., size, mobility, etc.) of a polypeptide of the invention complexed to a test compound from an unbound polypeptide of the invention can be used in the methods of the invention, including, but not limited to, electrophoresis, size exclusion chromatography, and mass spectrometry. Other methods to detect binding between polypeptide molecules of the invention and test compounds directly can also be used, including, but not limited to, affinity chromatography, scintillation proximity assay, nuclear magnetic resonance spectroscopy, and fluorescence resonance energy transfer.

In a first embodiment, electrophoresis is used to identify test compounds capable of binding a polypeptide of the invention. In general, a polypeptide molecule of the invention bound to a test compound is larger than an unbound polypeptide molecule of the invention. Electrophoretic separation based on size allows for determination of such a change in size. Any method of electrophoretic separation, including but not limited to, denaturing and non-denaturing polyacrylamide gel electrophoresis, urea gel electrophoresis, gel filtration, pulsed field gel electrophoresis, two dimensional gel electrophoresis, continuous flow electrophoresis, zone electrophoresis, agarose gel electrophoresis, and capillary electrophoresis can be used.

In a preferred embodiment, an automated electrophoretic system comprising a capillary cartridge having a plurality of capillary tubes is used for high-throughput screening of test compounds capable of binding a polypeptide of the invention. Such an apparatus for performing automated capillary gel electrophoresis is disclosed in U.S. Pat. Nos. 5,885,430; 5,916,428; 6,027,627; and 6,063,251.

In another preferred embodiment, the automated electrophoretic system can comprise a chip system consisting of complex designs of interconnected channels that perform and analyze enzyme reactions using part of a channel design as a tiny, continuously operating electrophoresis material, where reactions with one sample are going on in one area of the chip while electrophoretic separation of the products of another sample is taking place in a different part of the chip. Such a system is disclosed in U.S. Pat. Nos. 5,699,157; 5,842,787; 5,869,004; 5,876,675; 5,942,443; 5,948,227; 6,042,709; 6,042,710; 6,046,056; 6,048,498; 6,086,740; 6,132,685; 6,150,119; 6,150,180; 6,153,073; 6,167,910; 6,171,850; and 6,186,660. Briefly, the system disclosed in U.S. Pat. No. 5,699,157 provides for a microfluidic system for high-speed electrophoretic analysis of subject materials for applications in the fields of chemistry, biochemistry, biotechnology, molecular biology and numerous other areas. The system has a channel in a substrate, a light source and a photoreceptor. The channel holds subject materials in solution in an electric field so that the materials move through the channel and separate into bands according to species. The light source excites fluorescent light in the species bands and the photoreceptor is arranged to receive the fluorescent light from the bands. The system further has a means for masking the channel so that the photoreceptor can receive the fluorescent light only at periodically spaced regions along the channel. The system also has an unit connected to analyze the modulation frequencies of light intensity received by the photoreceptor so that velocities of the bands along the channel are determined, which allows the materials to be analyzed.

In another preferred embodiment, the electrophoretic method of separation comprises polyacrylamide gel electrophoresis, preferably non-denaturing the polyacrylamide gel electrophoresis, so as to differentiate the mobilities of the polypeptide molecules of the invention that are either unbound or bound to a test compound. In another embodiment, the polypeptides of the invention separated by the electrophoresis are transferred to a membrane for immunoblotting. Such techniques are well known to one of skill in the art.

In a second embodiment, size exclusion chromatography is used to identify test compounds capable of binding polypeptide molecules of the invention. Size-exclusion chromatography separates molecules based on their size and uses gel-based media comprised of beads with specific size distributions. When applied to a column, this media settles into a tightly packed matrix and forms a complex array of pores. Separation is accomplished by the inclusion or exclusion of molecules by these pores based on molecular size. Small molecules are included into the pores and, consequently, their migration through the matrix is retarded due to the added distance they must travel before elution. Large molecules are excluded from the pores and migrate with the void volume when applied to the matrix. In the present invention, a polypeptide molecule of the invention bound to a test compound will be larger, and thus elute faster from the size exclusion column, than an unbound polypeptide molecule.

In a third embodiment, mass spectrometry is used to identify test compounds capable of binding polypeptides of the invention. An automated method for analyzing mass spectrometer data which can analyze complex mixtures containing many thousands of components and can correct for background noise, multiply charged peaks and atomic isotope peaks is described in U.S. Pat. No. 6,147,344. The system disclosed in U.S. Pat. No. 6,147,344 is a method for analyzing mass spectrometer data in which a control sample measurement is performed providing a background noise check. The peak height and width values at each m/z ratio as a function of time are stored in a memory. A mass spectrometer operation on a material to be analyzed is performed and the peak height and width values at each m/z ratio versus time are stored in a second memory location. The mass spectrometer operation on the material to be analyzed is repeated a fixed number of times and the stored control sample values at each m/z ratio level at each time increment are subtracted from each corresponding one from the operational runs, thus producing a difference value at each mass ratio for each of the multiple runs at each time increment. If the MS value minus the background noise does not exceed a preset value, the m/z ratio data point is not recorded, thus eliminating background noise, chemical noise and false positive peaks from the mass spectrometer data. The stored data for each of the multiple runs is then compared to a predetermined value at each m/z ratio and the resultant series of peaks, which are now determined to be above the background, is stored in the m/z points in which the peaks are of significance.

In a fourth embodiment, affinity chromatography is used to identify test compounds capable of binding polypeptide molecules of the invention. To accomplish this, a polypeptide molecule of the invention is labeled with an affinity tag (e.g., GST, HA, myc, streptavidin, biotin) such that the polypeptide molecule of the invention can attach to a solid support through interaction with the affinity tag and solid support medium. The tagged polypeptide of the invention is contacted with a test compound either while free in solution or while bound to a solid support. The solid support is typically comprised of, but not limited to, cross-linked agarose beads that are coupled with a ligand for the affinity tag. Alternatively, the solid support may be a glass, silicon, metal, or carbon, plastic (polystyrene, polypropylene) surface with or without a self-assembled monolayer either with a covalently attached ligand for the affinity tag, or with inherent affinity for the tag on the polypeptide molecule of the invention.

Once the complex between the polypeptide molecule of the invention and test compound has reached equilibrium and has been captured, one skilled in the art will appreciate that the retention of bound compounds and removal of unbound compounds is facilitated by washing the solid support with large excesses of binding reaction buffer. Furthermore, retention of high affinity compounds and removal of low affinity compounds can be accomplished by a number of means that increase the stringency of washing; these means include, but are not limited to, increasing the number and duration of washes, raising the salt concentration of the wash buffer, addition of detergent or surfactant to the wash buffer, and addition of non-specific competitor to the wash buffer.

Following the removal of unbound compounds, bound compounds with high affinity for the immobilized polypeptide molecule of the invention can be eluted and analyzed. The elution of test compounds can be accomplished by any means that break the non-covalent interactions between the polypeptide of the invention and test compound. Means for elution include, but are not limited to, changing the pH, changing the salt concentration, the application of organic solvents, and the application of molecules that compete with the bound ligand. Preferably, the means employed for elution will release the compound from the polypeptide molecule of invention, but will not effect the interaction between the affinity tag and the solid support, thereby achieving selective elution of test compound.

In a preferred embodiment, affinity chromatography is used for high through put screening. In this embodiment, the test compound is detectably labeled (e.g., with fluorescent dyes, radioactive isotopes, etc.) and applied to polypeptide molecules of the invention in a spatially addressed fashion (e.g., attached to separate wells of a microplate). Binding between the test compound and the polypeptide molecule of the invention can be determined by the presence of the detectable label on the test compound to quickly identify which wells contain test compounds capable of binding.

In a fifth embodiment, a scintillation proximity assay (“SPA”) is used to identify test compounds capable of binding to a polypeptide of the invention. In this embodiment either the polypeptide of the invention or the test compound must labeled (e.g., with a radioisotope, etc.). The unlabeled entity is attached to a surface impregnated with a scintillant. The labeled entity is then incubated with the attached unlabeled entity under conditions that allow binding. The amount of binding between a polypeptide of the invention and test compound is quantitated with a scintillation counter (Cook, 1996, Drug Discov. Today 1:287-294; Mei et al., 1997, Bioorg. Med. Chem. 5:1173-1184; Mei et al., 1998, Biochemistry 37:14204-14212). High throughput SPA screening uses microplates with scintillant either directly incorporated into the plastic (Nakayama et al., 1998, J. Biomol. Screening 3:43-48) or coating the plastic. In a preferred embodiment, such microtiter plates are used in methods of the invention comprising (a) labeling of the polypeptide molecule of the invention with a radioactive label; (b) contacting the labeled polypeptide molecule with a test compound, wherein the test compound is attached to a microtiter well coated with scintillant; and (c) identifying and quantifying the amount of polypeptide of the invention bound to the test compound with SPA.

In a sixth embodiment, nuclear magnetic resonance spectroscopy (“NMR”) is used to identify test compounds capable of binding polypeptide molecules of the invention. NMR is used to identify polypeptide molecules of the invention that are bound by a test compound by qualitatively determining changes in chemical shift, specifically from distances measured using relaxation effects. NMR-based approaches have been used in the identification of small molecule binders of protein drug targets (Xavier et al., 2000, Trends Biotechnol. 18:349-356). A strategy for lead generation by NMR using a library of small molecules has been described (Fejzo et al., 1999, Chem. Biol. 6:755-769).

In a seventh embodiment, fluorescence resonance energy transfer (“FRET”) can be used to identify test compounds capable of binding to polypeptide molecules of the invention. In this embodiment, both the polypeptide molecule of the invention and the test compound are labeled with a different fluorescent molecule (i.e., flourophore). A characteristic change in fluorescence occurs when two fluorophores with overlapping emission and excitation wavelength bands are held together in close proximity, such as by a binding event. One of the fluorophores used as a label will have overlapping excitation and emission spectra with the other fluorophore used as a label such that one fluorophore (the donor) transfers its emission energy to excite the other fluorophore (the acceptor). The acceptor preferably emits light of a different wavelength upon relaxing to the ground state, or relaxes non-radioactively to quench fluorescence. FRET is very sensitive to the distance between the two fluorophores, and allows measurement of molecular distances less than 10 nm (e.g., U.S. Pat. No. 6,337,183 and Matsumoto et al., 2000, Bioorg. Med. Chem. Lett. 10:1857-1861).

5.6.1.2 Endogenous Binding Partners

Polypeptides which endogenously interact with a clk-2 polypeptide molecule of the invention in vivo can be identified by any method known in the art. Preferably, such endogenous binding partners participate in the same signaling cascade as clk-2 (e.g., are upstream or downstream from clk-2) or serve to modulate clk-2 expression/activity in vivo. Once identified, the expression or activity of clk-2 binding partners can be altered thus altering the clk-2 activity indirectly. Therefore, also encompassed by the methods of the invention are assays to identify agents which modulate the expression and/or activity of polypeptides which endogenously interact with clk-2.

One method which detects protein interactions in vivo, the two-hybrid system, is described in detail for illustration purposes only and not by way of limitation. One version of this system has been described (Chien et al., 1991, PNAS, 88:9578-9582) and is commercially available from Clontech (Palo Alto, Calif.). Briefly, utilizing such a system, plasmids are constructed that encode two hybrid proteins: one consists of the DNA-binding domain of a transcription activator protein fused to all or part of a polypeptide molecule of the invention, and the other consists of the activator protein's activation domain fused to an unknown protein that is encoded by a cDNA which has been recombined into this plasmid as part of a cDNA library. The plasmids are transformed into a strain of the yeast Saccharomyces cerevisiae that contains a reporter gene (e.g., lacZ) whose regulatory region contains the transcription activator's binding sites. Either hybrid protein alone cannot activate transcription of the reporter gene, the DNA-binding domain hybrid cannot because it does not provide activation function, and the activation domain hybrid cannot because it cannot localize to the activator's binding sites. Interaction of the two hybrid proteins reconstitutes the functional activator protein and results in expression of the reporter gene, which is detected by an assay for the reporter gene product.

The two-hybrid system or related methodology can be used to screen activation domain libraries for proteins that interact with a polypeptide molecule of the invention, which in this context is a “bait” gene product. Total genomic or cDNA sequences are fused to the DNA encoding an activation domain. This library and a plasmid encoding a hybrid of a polypeptide molecule of the invention coding region fused to the DNA-binding domain are co-transformed into a yeast reporter strain, and the resulting transformants are screened for those that express the reporter gene. These colonies are purified and the library plasmids responsible for reporter gene expression are isolated. DNA sequencing is then used to identify the proteins encoded by the library plasmids.

Another method to identify interacting proteins that may be in the same biological pathway as one or more polypeptides of the invention is by various expression analysis techniques to identify genes which are differentially expressed between two conditions, such as an animal or cell expressing a normal nucleic acid molecule of the invention compared to another animal or cell expressing a mutant nucleic acid molecule of the invention. Such techniques comprise any expression analysis technique known to one skilled in the art, including but not limited to differential display, serial analysis of gene expression (SAGE), nucleic acid array technology, subtractive hybridization, proteome analysis and mass-spectrometry of two-dimensional protein gels. In a specific embodiment, nucleic acid array technology (i.e., gene chips) may be used to determine a global (i.e., genome-wide) gene expression pattern in a normal animal or cell for comparison with an animal or cell having a mutation in one or more nucleic acid molecules of the invention.

To elaborate further, the various methods of gene expression profiling mentioned above can be used to identify other genes (or proteins) that may have a functional relation to (e.g., may participate in a signaling pathway with) a polypeptide molecule of the invention. Gene identification of such other genes is made by detecting changes in their expression levels following mutation, i.e., insertion, deletion or substitution in, or overexpression, underexpression, mis-expression or knock-out, of a polypeptide molecule of the invention. Expression profiling methods thus provide a powerful approach for analyzing the effects of mutation in a nucleic acid molecule of the invention.

Methods of gene expression profiling are well-known in the art, as exemplified by the following references describing subtractive hybridization (Wang & Brown, 1991, PNAS 88:11505-11509), differential display (Liang & Pardee, 1992, Science 257:967-971), SAGE (Velculescu et al., 1995, Science 270:484-487), proteome analysis (Humphery-Smith et al., 1997, Electrophoresis 18:1217-1242; Dainese et al., 1997, Electrophoresis 18:432-442), and hybridization-based methods employing nucleic acid arrays (Heller et al., 1997, PNAS 94:2150-2155; Lashkari et al., 1997, PNAS 94:13057-13062; Wodicka et al., 1997, Nature Biotechnol. 15:1259-1267).

5.6.2 In Vivo Assays

The invention also encompasses the use of an organism and/or cell in screening assays to identify agents of the invention. Agents that modulate the expression/activity of a clk-2 nucleic acid or polypeptide of the invention can be identified by contacting the organism or cell with a test compound and then determining if a change occurred in one or more clk-2 associated behaviors and/or phenotypes. In one embodiment, the cell or organism expresses a mutant clk-2 polypeptide that causes an altered behavior or phenotype as compared to an organism or cell expressing a wild type clk-2 polypeptide. Test compounds that are clk-2 agents of the invention will cause a change in at least one of the clk-2 associated behaviors and/or phenotypes. In a preferred embodiment, the change in clk-2 associated behavior and/or phenotype is such that it approximates (or is substantially similar) to that of an organism or cell expressing a wild type clk-2 polypeptide. In one embodiment, the organism used in screening assays, is a C. elegans. In another embodiment, the organism used in screening assays is a vertebrate, preferably a mammal, more preferably a mouse. In another embodiment, the organism used in screening assays is a mutant animal or a transgenic animal.

In a specific embodiment, a clk-2 agent of the invention which modulates the activity of a clk-2 polypeptide is identified comprising:

a) contacting a cell or organism with a compound, wherein the cell or organism exhibits at least one phenotype that is altered as a result of its expression of a mutant clk-2 polypeptide, when compared to a wild type cell or organism; and

b) determining the phenotype of said contacted cell or organism, wherein a difference in the phenotype of said contacted cell or organism as compared to the phenotype of a cell or organism expressing the mutant clk-2 polypeptide not contacted with the compound indicates that the compound modulates the activity of a clk-2 polypeptide.

Such screening methods can be employed using, for example, the C. elegans-based and cell-based assays as described below.

5.6.3 C. elegans-Based Assays

In certain embodiments, an agent of the invention that modulates a polypeptide molecule of the invention is identified by its ability to modulate a phenotype or behavior in an organism caused by the expression/activity of a polypeptide of the invention. In such embodiments, animal-based assays are used to quantitate such alterations in organism phenotype or behavior. In one embodiment, the organism-based assay is a C. elegans-based assay. In a specific embodiment, the C. elegans expresses a C. elegans polypeptide of the invention. In another specific embodiment, the C. elegans is transgenic (or recombinant) and expresses a polypeptide of the invention from a different species, preferably human. Such animals are used in the assays of the invention.

In specific embodiments, a test compound that modulates a clk-2 polypeptide molecule of the invention can be identified by its ability to modulate in C elegans the following phenotypes: telomere length, length of life, length of embryonic and post-embryonic development, frequency of defecation cycles, pharyngeal pumping rate, self-brood size, peak egg-laying rate, and proportion of dead eggs. Any method known in the art can be used to determine and quantitate the phenotypes used in screening (see e.g., Section 6).

5.6.4 Cell-Based Assays

In certain embodiments, an agent of the invention that modulates a polypeptide molecule of the invention is identified by its ability to modulate a cell phenotype caused by the expression/activity of a polypeptide of the invention. In such embodiments, in vitro cell-based assays are used to quantitate such alterations in phenotype. In one embodiment, the cell is a vertebrate or mammalian cell. In a preferred embodiment, human cells expressing human polypeptides of the invention are used in the assays.

5.6.4.1 Cell Growth

In one embodiment, an agent that modulates a clk-2 polypeptide molecule of the invention is identified by its ability to modulate cell growth. Many assays well-known in the art can be used to assess survival and/or growth; for example, cell proliferation can be assayed by measuring (3H)-thymidine incorporation, by direct cell count, by detecting changes in transcription, translation or activity of known genes such as cell cycle markers (Rb, cdc2, cyclin A, D1, D2, D3, E, etc). The levels of such protein and mRNA and activity can be determined by any method well known in the art. For example, protein can be quantitated by known immunodiagnostic methods such as western blotting or immunoprecipitation using commercially available antibodies (for example, many cell cycle marker antibodies are from Santa Cruz Inc.). mRNA can be quantitated by methods that are well known and routine in the art, for example by northern analysis, RNase protection, the polymerase chain reaction in connection with the reverse transcription, etc. Cell viability can be assessed by using trypan-blue staining or other cell death or viability markers known in the art.

The present invention provides for cell cycle and cell proliferation analysis by a variety of techniques known in the art, including but not limited to the following:

As one example, bromodeoxyuridine (BRDU) incorporation may be used as an assay to identify proliferating cells. The BRDU assay identifies a cell population undergoing DNA synthesis by incorporation of BRDU into newly synthesized DNA. Newly synthesized DNA may then be detected using an anti-BRDU antibody (see Hoshino et al., 1986, Int. J. Cancer 38:369; Campana et al., 1988, J. Immunol. Meth. 107:79).

Cell proliferation may also be examined using (3H)-thymidine incorporation (see e.g., Chen, 1996, Oncogene 13:1395-403; Jeoung, 1995, J. Biol. Chem. 270:18367-73). This assay allows for quantitative characterization of S-phase DNA synthesis. In this assay, cells synthesizing DNA will incorporate (3H)-thymidine into newly synthesized DNA. Incorporation may then be measured by standard techniques in the art such as by counting of radioisotope in a Scintillation counter (e.g. Beckman LS 3800 Liquid Scintillation Counter).

Detection of proliferating cell nuclear antigen (PCNA) may also be used to measure cell proliferation. PCNA is a 36 kilodalton protein whose expression is elevated in proliferating cells, particularly in early G1 and S phases of the cell cycle and therefore may serve as a marker for proliferating cells. Positive cells are identified by immunostaining using an anti-PCNA antibody (see Li et al., 1996, Curr. Biol. 6:189-99; Vassilev et al., 1995, J. Cell Sci. 108:1205-15).

Cell proliferation may be measured by counting samples of a cell population over time (e.g. daily cell counts). Cells may be counted using a hemacytometer and light microscopy (e.g. HyLite hemacytometer, Hausser Scientific). Cell number may be plotted against time in order to obtain a growth curve for the population of interest. In a preferred embodiment, cells counted by this method are first mixed with the dye Trypan-blue (Sigma), such that living cells exclude the dye, and are counted as viable members of the population.

DNA content and/or mitotic index of the cells may be measured, for example, based on the DNA ploidy value of the cell. For example, cells in the G1 phase of the cell cycle generally contain a 2N DNA ploidy value. Cells in which DNA has been replicated but have not progressed through mitosis (e.g. cells in S-phase) will exhibit a ploidy value higher than 2N and up to 4N DNA content. Ploidy value and cell-cycle kinetics may be further measured using propidum iodide assay (see e.g. Turner, et al., 1998, Prostate 34:175-81). Alternatively, the DNA ploidy may be determined by quantitation of DNA Feulgen staining (which binds to DNA in a stoichiometric manner) on a computerized microdensitometrystaining system (see e.g., Bacus, 1989, Am. J. Pathol. 135:783-92). In an another embodiment, DNA content may be analyzed by preparation of a chromosomal spread (Zabalou, 1994, Hereditas. 120:127-40; Pardue, 1994, Meth. Cell Biol. 44:333-351).

The expression of cell-cycle proteins (e.g., CycA. CycB, CycE, CycD, cdc2, Cdk4/6, Rb, p21, p27, etc.) provide crucial information relating to the proliferative state of a cell or population of cells. For example, identification in an anti-proliferation signaling pathway may be indicated by the induction of p21^{cip1}. Increased levels of p21 expression in cells results in delayed entry into G1 of the cell cycle (Harper et al., 1993, Cell 75:805-816; Li et al., 1996, Curr. Biol. 6:189-199). p21 induction may be identified by immunostaining using a specific anti-p21 antibody available commercially (e.g. Santa Cruz). Similarly, cell-cycle proteins may be examined by western blot analysis using commercially available antibodies. In another embodiment, cell populations are synchronized prior to detection of a cell cycle protein. Cell cycle proteins may also be detected by FACS (fluorescence-activated cell sorter) analysis using antibodies against the protein of interest.

clk-2 agents of the invention can also be identified by their ability to change the length of the cell cycle or speed of cell cycle so that cell proliferation is decreased or inhibited. In one embodiment the length of the cell cycle is determined by the doubling time of a population of cells (e.g., using cells contacted or not contacted with one or more test compounds). In another embodiment, FACS analysis is used to analyze the phase of cell cycle progression, or purify GI, S, and G2/M fractions (see e.g., Delia et al., 1997, Oncogene 14:2137-47).

5.6.4.2 Apoptosis

In another embodiment, an agent that modulates a clk-2 polypeptide of the invention is identified by its ability to modulate apoptosis, especially apoptosis stimulated by oxidative stress or inhibition of DNA synthesis.

Oxidative stress and DNA synthesis inhibition can be accomplished by any method known in the art. In one embodiment, DNA synthesis inhibition is caused by hydroxyurea treatment. In another embodiment, oxidative stress is caused by menadione or t-butyl hydroperoxide treatment (Jamieson et al., 1994, Microbiology 140:3277-3283).

Apoptosis is associated with a number of morphological and biochemical alterations. Morphological alterations characteristic of apoptosis are well known in the art and include, e.g., condensed and rounded cellular morphology, membrane blebbing, the formation of apoptotic bodies (i.e., membrane-bound bodies containing cytoplasmic and nuclear components), and condensation of the nucleus with cytoplasmic organelles being relatively well maintained (Studzinski (Ed.), Cell Growth and Apoptosis, Oxford: Oxford University Press (1995)). Biochemical alterations characteristic of apoptosis also are well known in the art. The classical biochemical alteration characteristic of apoptosis is the appearance of oligonucleosome-sized fragments of DNA, which produce a “ladder” upon agarose gel electrophoresis. This extensive fragmentation can be preceded by an earlier endonucleolytic cleavage of chromatin, producing DNA fragments of about 50 kb to 300 kb in size.

A variety of assays for determining whether a test compound can promote or inhibit clk-2-mediated apoptosis are well known in the art. Such methods include light microscopy for determining the presence of one or more morphological characteristics of apoptosis, such as condensed or rounded morphology, shrinking and blebbing of the cytoplasm, preservation of structure of cellular organelles including mitochondria, and condensation and margination of chromatin. Biochemical indicators of apoptosis can be assayed by a number of methods, such as using terminal deoxytransferase-mediated (TdT) dUTP biotin nick end-labeling (TUNEL) (Gavriel et al., 1992, J. Cell Biol. 119:493; Gorczyca et al., 1992, Int. J. Oncol 1:639; Desjardins & MacManus, 1995, Exp Cell Res 216:380-387), digoxygenin labeling using APOPTAG (commercially available from ONCOR, Inc.; Gaithersburg Md.), and detection of nucleosomal DNA fragments using agarose gel electrophoresis (Studzinski (Ed.), Cell Growth and Apoptosis, Oxford: Oxford University Press (1995); Gong et al., 1994, Anal. Biochem. 218:314). DNA filter elution methodology also can be used to detect apoptosis-associated DNA fragmentation and to determine apoptotic or anti-apoptotic activity (Studzinski (Ed.), Cell Growth and Apoptosis, Oxford: Oxford University Press (1995); Bertrand et al., 1995, Drug Devel. 34:138). Apoptotic or anti-apoptotic activity also can be detected and quantitated by determining an altered mitochondrial to nuclear DNA ratio as described in, e.g., Tepper et al., 1992, Anal. Biochem. 203:127 and Tepper & Studzinski, 1993, J. Cell Biochem. 52:352. See generally, Apoptosis Detection and Assay Methods, 1998, Zhu & Chun eds, Eaton Publishing, Natick, MA for protocols to detect and measure apoptosis (including, but not limited to, annexin V assay, single-strand DNA antibody assays, caspase substrate assay, etc.). One skilled in the art understands that these, or other assays for apoptotic or anti-apoptotic activity, can be performed using routine methodology.

5.6.4.3 Telomere Length

In one embodiment, an agent that modulates a clk-2 polypeptide molecule of the invention is identified by its ability to modulate telomere length. Any method known in the art to detect telomere length can be used.

Probe-Based Methods for Measuring Telomere Length

Telomere length can be determined using cell lysates (see e.g., U.S. Pat. Nos. 5,834,198 and 5,645,986). Chromosomal DNA is isolated from cells in which telomeres are to be measured by standard DNA extraction procedures. The chromosomal DNA is then denatured and hybridized to a labeled an oligonucleotide probe having a sequence complementary to a telomere repeat sequence. The amount of bound probe is measured and correlated to telomere length using standards of known length or conversion factors to convert the amount of bound probe to a measure of telomere length. In these probe-based methods, the probe is added in excess, so that all or substantially all of the telomeric repeats in the telomere are hybridized to the probe.

In another embodiment, the invention provides a method of measuring telomere length in which the chromosomal DNA is cross-linked to a solid phase. In this embodiment, genomic DNA is spotted on and cross-linked to a solid support (e.g., a nitrocellulose filter) using UV irradiation. Typically, the chromosomal DNA is sheared or cleaved into smaller fragments prior to binding to the solid phase. The genomic DNA is cleaved or sheared enzymatically or mechanically, e.g., with restriction endonucleases, sonication, or other methods known in the art. The amount of labeled oligonucleotide probe having a sequence complementary to a telomere repeat sequence hybridized to the nucleic acid in each position on the solid support is quantitated, and the quantitated amount is correlated with telomere length, e.g., by comparing to a standard.

In another embodiment, one measures the loss of or decrease in bound probe observed after treatment of the genomic DNA with a known amount of exonuclease that degrades DNA specifically from the ends of the chromosome to measure telomere length. A preferred exonuclease is Bal31, an exonuclease that digests single- or double-stranded DNA specifically from the end of a DNA, such as the end of a telomere. The rate of Bal31 digestion is about 50 bp/min. Thus, when chromosomal DNA is digested with Bal31, DNA internal to the telomeres is digested last while telomeric DNA is digested first. The method can be conveniently carried out by spotting Bal31 enzyme (e.g., serial dilutions) on a DNA binding membrane, e.g., nitrocellulose, binding chromosomal DNA to the membrane, incubating under conditions where the nuclease enzyme is active for a specific period of time, denaturing the enzyme and DNA, and hybridizing the remaining DNA to an oligonucleotide probe having a sequence complementary to a telomere repeat sequence. The amount of probe hybridization, which should decrease with increasing Bal31 concentration or reaction time, can be used to determine the telomere length. If desired, telomeres of known length or cells comprising telomeres of known length can be used as standards.

In another embodiment, telomere length can be determined in whole cells. Fluorescence in situ hybridization (FISH) can be used to measure telomere length in whole cells. Whole cells are attached to a solid support or surface before cell permeabilization. The cellular DNA is denatured and a fluorescein-labeled telomere probe (or a mixture of labeled probes) is added and hybridized to the telomeric repeats in the denatured DNA. Cells are analyzed using confocal microscopy. FISH analysis of cells can be used for a variety of purposes: to determine average relative telomere lengths in a population of cells, to determine the longest telomere length in a population of cells, to determine changes in telomere length in a cell population over time or after treatment with an agent or exposure to certain conditions.

Flow Cytometry for Measuring Telomere Length

Telomere length of the chromosomes of one or more cells can be measured using flow cytometry (see e.g, U.S. Pat. No. 5,834,198). This methodology also allows cells to be sorted based on telomere length. Growing cells are harvested by trypsinization and washed in PBS as per standard procedures. The washed cells are then fixed. The fixed cells are centrifuged and washed three times with PBS. Cells are then treated with RNase A for 20 minutes at 37° C. followed by pepsin treatment for 5 minutes at 37° C. The cells are centrifuged and then resuspended in hybridization buffer composed of 70% deionized formamide containing FITC-labeled peptide nucleic acid probe (PNA see e.g., Corey, 1997, Trends Biotechnol. 15:224-9). The PNA probe, sonicated salmon sperm DNA (or other commercially available reagent to prevent non-specific probe hybridization), and 10 mM Tris pH 7.2 are incubated at room temperature for 2-8 hours. Because all cells fluoresce to some degree, a control experiment is conducted under the same conditions, except that the PNA probe is unlabeled to determine background fluorescence. After the hybridization step, the cells are washed to remove unbound probe and resuspended in PBS for analysis with a flow cytometer.

For analysis with a flow cytometer, a standard optics and filter arrangement for a FITC-generated signal is used (488 nm excitation, 525 nm bandpass filter for emission). The signals to be collected include log and linear FITC fluorescence (525 nm) and light scatter (0° angle and 90° angle) as correlated parameters. During flow cytometry, cell pass a laser at a wavelength which generates scattered light and fluorescence signals from the cells. The photomultiplier tube detects the generated photons, and the signal is passed through a digital-to-analog converter. The resultant signals can be displayed either linearly or logarithmically. Logarithmic displays provide better separation of the peaks, whereas linear displays generally provide more sensitivity.

The cells can also be counterstained with a DNA specific dye such as propidium iodide to measure cellular DNA content simultaneously. If counterstaining is used, the same filter set-up as described above is used (the PI signal is measured using a 610 nm bandpass filter). This set-up will allow determination of cell cycle position and cellular DNA content, as well as quantitation of the hybridized probe signal. The intensity of signal from bound probe per cell is proportional to the number of telomeric repeats and to the telomere length. As the signal intensity is measured, the instrument can be programmed to deflect the cells into specific tubes based upon the signal and the corresponding telomere length.

PCR-Based Measurement of Telomere Length

Telomere length can be determined by a PCR-based method (see e.g., U.S. Pat. Nos. 5,834,198; 5,741,677; 5,645,986). The telomeric DNA is first treated with an exonuclease to generate blunt ends, and then, a double-stranded linker is attached to the 3′ end of the telomere. A forward primer complementary to the linker and a subtelomeric return primer complementary to the subtelomeric region of a chromosome are extended by PCR in the presence of nucleotide triphosphates. The long PCR primer extension products are then separated by size on a gel, and size standards on the gel are used to determine telomere length.

Modified Maxam-Gilbert Reaction to Measure Telomere Length

Telomere length can be measured with a modified Maxam-Gilbert reaction (see e.g., U.S. Pat. No. 5,645,986). This method can be best be understood by distinguishing it from a classic Southern blot method to measure telomere length. Genomic DNA is digested with a restriction enzyme with a four-base recognition sequence (e.g., Hi-fi) before separation of the fragments by electrophoresis. The DNA fragments are transferred to a membrane via a Southern blot and then hybridized to a radiolabeled probe that hybridizes telomeric sequences (TTAGGG)₃ (SEQ. ID NO. 59). A difficulty with this classic method is that the resulting terminal restriction fragments contain a 3-5 kb stretch of subtelomeric DNA that lacks restriction sites and thereby adds significantly to the size of the measured telomere length.

The modified Maxam-Gilbert reaction eliminates this sub-telomeric DNA and improves accuracy of telomere length determination by utilizing the fact that the subtelomeric DNA contains G and C residues in both strands, and thus should be cleaved under conditions that cause breaks at G residues. In contrast, DNA composed exclusively of telomeric repeats will have one strand lacking G residues, and this strand should remain intact under G-cleavage conditions.

The Maxam-Gilbert G-reaction uses piperidine to cleave guanine residues that have been methylated by dimethylsulfate (DMS) treatment. Although the original conditions of the Maxam-Gilbert G-reaction (treatment in IM piperidine for 30 min. at 90° C.) breaks unmethylated DNA into fragments of 1-2 kb (and is thus non-specific), milder conditions (0.1M piperidine for 30 min. at 37° C.) leave untreated DNA intact. The DNA is therefore treated with DMS and piperidine, precipitated with ethanol, electrophoresed, Southern blotted, and hybridized to a labeled telomeric probe.

5.6.5 Changes in Gene Expression

In one embodiment, agents of the invention are identified based on the ability to alter expression of a nucleic acid or polypeptide molecule of the invention in vivo. Assays for changes in gene expression are well known in the art (see e.g., PCT International Publication No. WO 96/34099). In particular, the assays may detect the presence of increased or decreased expression of a nucleic acid or protein of the invention on the basis of increased or decreased mRNA expression (using, e.g., nucleic acid probes), increased or decreased levels of related protein products (using, e.g., antibodies), or increased or decreased levels of expression of a reporter gene (see e.g. Section 5.3.1) operably associated with a 5′ regulatory region in a recombinant construct. Such assays can be performed with animals (i.e., C. elegans) or in cells in tissue culture.

In one specific embodiment, a clk-2 agent of the invention which modulates the expression of a clk-2 nucleic acid or polypeptide is identified comprising:

a) contacting a cell with a compound, and

b) determining the level of expression of the clk-2 nucleic acid or polypeptide in said contacted recombinant cell,

wherein a difference in the expression level of the clk-2 nucleic acid or polypeptide in said contacted recombinant cell as compared to the expression level of the clk-2 nucleic acid or polypeptide in said recombinant cell not contacted with the compound indicates that the compound modulates clk-2 expression.

In another specific embodiment, a clk-2 agent of the invention which modulates the expression of a clk-2 polypeptide is identified comprising:

a) contacting a recombinant cell with a compound, said recombinant cell comprising a reporter gene operably associated with a regulatory sequence of a clk-2 gene, such that expression of the reporter gene is regulated by the regulatory sequence; and

b) determining the level of expression of said reporter gene in said contacted recombinant cell,

wherein a difference in the expression level of said reporter gene in said contacted recombinant cell as compared to the expression level of said reporter gene in said recombinant cell not contacted with the compound indicates that the compound modulates clk-2 expression.

5.6.6 Changes in Phosphorylation

In one embodiment, agents of the invention are identified based on the ability to alter the level of phosphorylation of clk-2. As shown in Section 8, mclk-2 is post-translationally modified by phosphorylation. The inventors believe that hclk-2 is also phosphorylated and that the degree of phosphorylation and the status of phosphorylation site(s) on the protein affects the activity of clk-2. Accordingly, the invention encompasses a strategy for modulating the activity of clk-2 by modulation of the level of clk-2 phosphorylation. Thus, a clk-2 agent that is based on modulating clk-2 phosphorylation status is provided.

The level of clk-2 phosphorylation can be increased by enhancing the activity of kinases that phosphorylate clk-2 or by decreasing the activity of phosphatases that dephosphorylate clk-2. Thus, for example, both kinase enhancers and phosphatase inhibitors that affect the level of clk-2 phosphorylation are contemplated for uses as an a clk-2 agent.

The level of clk-2 phosphorylation can be decreased by decreasing the activity of kinases that phosphorylate clk-2 or by enhancing the activity of phosphatases that dephosphorylate clk-2. Thus, for example, the invention also includes both kinase inhibitors and phosphatase enhancers that affect the level of clk-2 phosphorylation. In one embodiment, a kinase inhibitor interferes with the ability of the kinase to bind a natural binding partner (e.g., substrate, ATP, etc.).

The level of clk-2 phosphorylation can also be altered by inhibiting or promoting interactions of clk-2 with kinases and/or phosphatases which normally interact with clk-2. In this embodiment, the invention provides an agent that imposes steric restrictions to or blocks access to one or more phosphorylation sites on clk-2, e.g., antibodies directed to clk-2 that block phosphorylation or dephosphorylation. Anti-clk2 antibodies can be made such that only certain specific phosphorylation sites are bound, and that antibody binding can be conditional upon the presence or absence of a phosphate group.

In one specific embodiment, candidate agents are incubated with cells expressing clk-2. A clk-2 agent of the invention which modulates the phosphorylation level of a clk-2 polypeptide is identified comprising contacting a cell or organism with a compound, wherein the cell or organism expresses clk-2; and determining the phosphorylation level of clk-2 in said contacted cell or organism, wherein a difference in the phosphorylation level of clk-2 in said contacted cell or organism as compared to the phosphorylation level of clk-2 in a cell or organism not contacted with said compound indicates that the compound modulates the phosphorylation level of a clk-2 polypeptide.

In another specific embodiment, candidate agents are incubated with clk-2 outside of a cell. A clk-2 agent of the invention which modulates the phosphorylation level of a clk-2 polypeptide is identified comprising contacting a reaction mixture with a compound, said mixture comprising clk-2 and at least one polypeptide able to phosphorylate or dephosphorylate clk-2; and determining the phosphorylation level of clk-2 in said mixture, wherein a difference in the phosphorylation level of clk-2 as compared to the phosphorylation level of clk-2 in a mixture not contacted with said compound indicates that the compound modulates the phosphorylation level of a clk-2 polypeptide. Clk-2 protein can be omitted in the initial incubation with the compound in which case it is added later to the reaction mixture.

Candidate agents for this type of assays are preferably serine/threonine kinase inhibitors, tyrosine kinase inhibitors, and inhibitors for phosphatases that act on phosphorylated serine, threonine, and/or tyrosine. Examples of candidate compounds include, but are not limited to, phenylaminopyrimidine tyrosine kinase inhibitors e.g., 2-phenylaminopyrimidine; pyrimidinyl pyridione tyrosine kinase inhibitors; 2-(Purin-9-yl)-tetrahydrofuran-3,4-diol derivatives; pyridoxine and pyridoxal analogues; N-6 heterocyclic 8-modified adenosine derivatives; N-6 heterocyclic 5′-modified adenosine derivatives; allosteric inhibitors of pyruvate kinase; 8-phenylxanthines, 8-cycloalkylxantines or 8-substituted xanthine derivatives; N-6 substituted adenosine-5′-uronamides; purine, pyrrolo [2,3,d]pyrimidine and pyrazolo [3,4,d]pyrimidine nucleoside analogs; heterocyclic-hydroxyimino-fluorene nuclei compounds; 3-anilinomethylene oxindoles; 3-(4′-bromobenzylindenyl)-2-indolinone analogues; indeno[1,2,c]-naphthol[1,2,c] and benzo[6,7]cyclohepta[1,2,c]pyrazole derivatives; 3′-epimeric k-252a derivatives; quinazolines; 3-cyano-[1,7], [1,5] and [1,8]-napthyridine analogues; -[4-(3-chloro-4-fluoro-phenylamino)-7-(3-morpholin4-yl-propoxy)-quinazolin-6-yl]-acrylamide; pyrimidine derivatives; benzimidazoles; bicyclic heteroaromatic compounds; pyrrolopyrimidines; quinoline and quinoxaline derivatives; indolinones; 2-pyrimidineamine derivatives; substituted pyrido [3,2,d]pyrimidines; fused polycyclic 2-aminopyrimidine derivatives; bicyclic 4-aralkylaminopyrimidine derivatives; N-7-heterocycyl pyrrolo [2,3,d]pyrimidines; 3-cyano quinoline derivatives; pyrazole derivatives; pyrimido [5,4,d]pyrimidines; 4-anilinoquinazoline derivatives; 6-aryl napthyridines; oxides of amino containing pyrido [2,3,d]pyrimidines; 5-aminopyrazoles; 5,10-dihydropyrimido[4,5,b]quinolin-4(1H)-one; quinolymethylen-oxindole analogues; acrylonitrile-sulfonamide derivatives; 3-(4′-dimethylaminobenzylidenyl)-2-indolines; 3-(2′-alkoxybenzylidenyl)2-indolines; 3-(4′-bromobenzylidenyl)-2-indolines; benzylidene-Z-indoline compounds; 4,6-dianilino-pyrimidine derivatives; substituted indolylmethyleneoxindole analogues; hydrosoluble 3-arylidene-2-oxindole analogues; 3-(2′-halobenzylidenyl)-2-indolinone compounds; 3-heteroaryl-2-indolinone compounds; benzoylethylene derivatives; urea and thiourea-type compounds; benzopyran derivatives; pyrido[2,3,d]pyrimidines; 6-aryl-pyrido[2,3,d]pyrimidines and naphthyridines; substituted 3-arylidene-7-azaoxindole compounds; thienyl compounds; aryl and heteroaryl quinazoline compounds; arylidene and heteroarylidene oxindole derivatives; N-substituted-beta-aryl and beta-heteroaryl-alpha-cyanoacrylamide derivatives; 2-iminochromene derivatives; 4-aminopyrrolo [2,3,d]pyrimidines; 4-aminopyrazolo(3,4,d)pyrimidine derivatives; 4-aminopyrazolo(3,4,d)pyridine derivatives; 3-(cycloalkanoheteroarylidenyl)-2-indolinones; isoxazole-4-carboxamide compounds; 3-(cycloalkanoheteroarylidenyl)-2-indolinones; substituted phenylacrylonitrile compounds; benzylidene-Z-indoline compounds; 3-(2′-halobenzyliclenyl)-2-indolinone compounds; benzopyran compounds; 4-aminopyrimidines; isoxazole compounds; STI-571; mesylate salt of STI-571 (imatinib mesylate, GLEEVEC™, Novartis Pharmaceuticals), and congeners thereof; members of the 2-phenylaminopyrimidine class of compounds; pyridione tyrosine kinase inhibitors.

Assays for determining phosphorylation levels are well known in the art. See, for example, methods described in Protein Phosphorylation: A Practical Approach, 2^{nd} edition, 1999, edited by G. Hardie, Oxford University Press, England. For example, altered phosphorylation levels result in altered size or mobility of a polypeptide (see, e.g., Section 8.2.2). Any method that detects an altered size or mobility of clk-2 can be used in the methods of the invention to detect altered phosphorylation levels, including, but not limited to, electrophoresis, size exclusion chromatography, and mass spectrometry (as described in Section 5.6.1.1).

5.7 Nucleic Acid-Based Therapeutics

As described above, nucleic acid molecules can be clk-2 agents of the invention. Nucleic acid molecules can be used to decrease clk-2 expression and, therefore, be used in methods of the invention.

5.7.1 Antisense Nucleic Acids

The present invention encompasses antisense nucleic acid molecules, i.e., molecules which are complementary to all or part of a sense nucleic acid encoding clk-2, e.g., complementary to the coding strand of a double-stranded cDNA molecule or complementary to an mRNA sequence. Accordingly, an antisense nucleic acid can hydrogen bond to a sense nucleic acid. The antisense nucleic acid can be complementary to an entire coding strand, or to only a portion thereof, e.g., all or part of the protein coding region (or open reading frame). An antisense nucleic acid molecule can be antisense to all or part of a non-coding region of the coding strand of a nucleotide sequence encoding a polypeptide of the invention. The non-coding regions (“5′ and 3′ untranslated regions”) are the 5′ and 3′ sequences which flank the coding region and are not translated into amino acids. In one embodiment, the antisense nucleic acid molecule is complementary to all or part of clk-2 (e.g., SEQ ID NOs:1, 4, 5, 6, 7, 15, 16, 20, 21, 22, 23, 24).

An antisense oligonucleotide can be, for example, about 5, 10, 15, 20, 25, 30, 35, 40, 45 or 50 nucleotides in length. An antisense nucleic acid of the invention can be constructed using chemical synthesis and enzymatic ligation reactions using procedures known in the art. For example, an antisense nucleic acid (e.g., an antisense oligonucleotide) can be chemically synthesized using naturally occurring nucleotides or variously modified nucleotides designed to increase the biological stability of the molecules or to increase the physical stability of the duplex formed between the antisense and sense nucleic acids, e.g., phosphorothioate derivatives and acridine substituted nucleotides can be used. Examples of modified nucleotides which can be used to generate the antisense nucleic acid include 5-fluorouracil, 5-bromouracil, 5-chlorouracil, 5-iodouracil, hypoxanthine, xanthine, 4-acetylcytosine, 5-(carboxyhydroxylmethyl) uracil, 5-carboxymethylaminomethyl-2-thiouridine, 5-carboxymethylaminomethyluracil, dihydrouracil, β-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil, β-D-mannosylqueosine, 5′-methoxycarboxymethyluracil, 5-methoxyuracil, 2-methylthio-N-6-isopentenyladenine, uracil-5-oxyacetic acid (v), wybutoxosine, pseudouracil, queosine, 2-thiocytosine, 5-methyl-2-thiouracil, 2-thiouracil, 4-thiouracil, 5-methyluracil, uracil-5-oxyacetic acid methylester, uracil-5-oxyacetic acid (v), 5-methyl-2-thiouracil, 3-(3-amino-3-N-2-carboxypropyl) uracil, (acp3)w, and 2,6-diaminopurine. Alternatively, the antisense nucleic acid can be produced biologically using an expression vector into which a nucleic acid has been subcloned in an antisense orientation (i.e., RNA transcribed from the inserted nucleic acid will be of an antisense orientation to a target nucleic acid of interest, i.e., clk-2).

The antisense nucleic acid molecules of the invention are typically administered to a subject or generated in situ such that they hybridize with or bind to cellular mRNA and/or genomic DNA encoding a selected polypeptide of the invention to thereby inhibit expression, e.g, by inhibiting transcription and/or translation. The hybridization can be by conventional nucleotide complementarity to form a stable duplex, or, for example, in the case of an antisense nucleic acid molecule which binds to DNA duplexes, through specific interactions in the major groove of the double helix.

An antisense nucleic acid molecule of the invention can be an a-anomeric nucleic acid molecule. An α-anomeric nucleic acid molecule forms specific double-stranded hybrids with complementary RNA in which, contrary to the usual β-units, the strands run parallel to each other (Gaultier et al., 1987, Nucleic Acids Res. 15:6625). The antisense nucleic acid molecule can also comprise a 2′-o-methylribonucleotide (Inoue et al., 1987, Nucleic Acids Res. 15:6131) or a chimeric RNA-DNA analogue (Inoue et al., 1987, FEBS Lett. 215:327).

5.7.2 Ribozymes

The invention also encompasses ribozymes. Ribozymes are catalytic RNA molecules with ribonuclease activity which are capable of cleaving a single-stranded nucleic acid, such as an mRNA, to which they have a complementary region. Thus, ribozymes (e.g., hammerhead ribozymes; described in Haselhoff and Gerlach, 1988, Nature 334:585-591) can be used to catalytically cleave mRNA transcripts to thereby inhibit translation of the protein encoded by the mRNA. A ribozyme having specificity for a nucleic acid molecule encoding a polypeptide of the invention (i.e., clk-2) can be designed based upon the nucleotide sequence of the polypeptide of the invention (i.e., clk-2). For example, a derivative of a Tetrahymena L-19 IVS RNA can be constructed in which the nucleotide sequence of the active site is complementary to the nucleotide sequence to be cleaved in U.S. Pat. Nos. 4,987,071 and 5,116,742. Alternatively, an mRNA encoding a polypeptide of the invention can be used to select a catalytic RNA having a specific ribonuclease activity from a pool of RNA molecules. See, e.g., Bartel and Szostak, 1993, Science 261:1411.

5.7.3 RNA Interference

In certain embodiments, an RNA interference (RNAi) molecule is used to decrease clk-2 expression. RNA interference (RNAi) refers to the use of double-stranded RNA (dsRNA) or small interfering RNA (siRNA) to suppress the expression of a gene comprising a related nucleotide sequence. RNAi is also called post-transcriptional gene silencing (or PTGS). Since the only RNA molecules normally found in the cytoplasm of a cell are molecules of single-stranded mRNA, the cell has enzymes that recognize and cut dsRNA into fragments containing 21-25 base pairs (approximately two turns of a double helix and which are referred to as small interfering RNA or siRNA). The antisense strand of the fragment separates enough from the sense strand so that it hybridizes with the complementary sense sequence on a molecule of endogenous cellular mRNA. This hybridization triggers cutting of the mRNA in the double-stranded region, thus destroying its ability to be translated into a polypeptide. Introducing dsRNA corresponding to a particular gene thus knocks out the cell's own expression of that gene in particular tissues and/or at a chosen time.

Double-stranded (ds) RNA can be used to interfere with gene expression in mammals. dsRNA is used as inhibitory RNA or RNAi of the function of a nucleic acid molecule of the invention to produce a phenotype that is the same as that of a null mutant of a nucleic acid molecule of the invention (Wianny & Zernicka-Goetz, 2000, Nature Cell Biology 2: 70-75).

Alternatively, siRNA can be introduced directly into a cell to mediate RNA interference (Elbashir et al., 2001, Nature 411:494-498). Many methods have been developed to make siRNA, e.g, chemical synthesis or in vitro transcription. Once made, the siRNAs are introduced into cells via transient transfection. See also U.S. Patent Applications 60/265,232, Ser. No. 09/821,832 and PCT/US01/10188, directed to RNA Sequence-Specific Mediators of RNA Interference. A number of expression vectors have also been developed to continually express siRNAs in transiently and stably transfected mammalian cells (Brummelkamp et al., 2002 Science 296:550-553; Sui et al., 2002,. PNAS 99(6):5515-5520; Paul et al., 2002, Nature Biotechnol. 20:505-508). Some of these vectors have been engineered to express small hairpin RNAs (shRNAs), which get processed in vivo into siRNA-like molecules capable of carrying out gene-specific silencing. Another type of siRNA expression vector encodes the sense and antisense siRNA strands under control of separate pol III promoters (Miyagishi and Taira, 2002, Nature Biotechnol. 20:497-500). The siRNA strands from this vector, like the shRNAs of the other vectors, have 5′ thymidine termination signals. Silencing efficacy by both types of expression vectors was comparable to that induced by transiently transfecting siRNA.

5.7.4 Gene Therapy

In a specific embodiment, nucleic acids of the invention (e.g., clk-2 antisense nucleic acids, clk-2 dsRNA, clk-2 ribozymes, or nucleic acids that encode a clk-2 intrabody) are administered to treat, prevent or manage a disorder by way of gene therapy. Gene therapy refers to therapy performed by the administration to a subject of an expressed or expressible nucleic acid. In this embodiment of the invention, the nucleic acids mediate a prophylactic or therapeutic effect.

Any of the methods for gene therapy available in the art can be used according to the present invention. Exemplary methods are described below.

For general reviews of the methods of gene therapy, see Goldspiel et al., 1993, Clinical Pharmacy 12:488; Wu and Wu, 1991, Biotherapy 3:87; Tolstoshev, 1993, Ann. Rev. Pharmacol. Toxicol. 32:573; Mulligan, 1993, Science 260:926-932; and Morgan and Anderson, 1993, Ann. Rev. Biochem. 62:191; May, 1993, TIBTECH 11:155. Methods commonly known in the art of recombinant DNA technology which can be used are described in Ausubel et al. (eds.), Current Protocols in Molecular Biology, John Wiley & Sons, NY (1993); and Kriegler, Gene Transfer and Expression, A Laboratory Manual, Stockton Press, NY (1990).

In a preferred aspect, a composition of the invention comprises a nucleic acid of the invention (e.g.,encode an antisense or intrabody molecule), said nucleic acid being part of an expression vector that expresses the nucleic acid in a suitable host. In particular, such nucleic acids have promoters, preferably heterologous promoters, said promoter being inducible or constitutive, and, optionally, tissue-specific. In another particular embodiment, nucleic acid molecules used comprise nucleic acid molecules of the invention flanked by regions that promote homologous recombination at a desired site in the genome, thus providing for intrachromosomal expression of the nucleic acids of the invention (Koller and Smithies, 1989, PNAS 86:8932; Zijlstra et al., 1989, Nature 342:435).

Delivery of the nucleic acids into a subject may be either direct, in which case the subject is directly exposed to the nucleic acid or nucleic acid-carrying vectors, or indirect, in which case, cells are first transformed with the nucleic acids in vitro, then transplanted into the subject. These two approaches are known, respectively, as in vivo or ex vivo gene therapy. In a specific embodiment, the nucleic acid sequences are directly administered in vivo. This can be accomplished by any of numerous methods known in the art, e.g., by constructing them as part of an appropriate nucleic acid expression vector and administering it so that they become intracellular, e.g., by infection using defective or attenuated retrovirals or other viral vectors (see e.g., U.S. Pat. No. 4,980,286), or by direct injection of naked DNA, or by use of microparticle bombardment (e.g., a gene gun; Biolistic, Dupont), or coating with lipids or cell-surface receptors or transfecting agents, encapsulation in liposomes, microparticles, or microcapsules, or by administering them in linkage to a peptide, e.g., through a thioester bond, which is known to enter the cell (e.g., a membrane permeable sequence) and/or nucleus, by administering it in linkage to a ligand subject to receptor-mediated endocytosis (see, e.g., Wu and Wu, 1987, J. Biol. Chem. 262:4429) (which can be used to target cell types specifically expressing the receptors), etc. In another embodiment, nucleic acid-ligand complexes can be formed in which the ligand comprises a fusogenic viral peptide to disrupt endosomes, allowing the nucleic acid to avoid lysosomal degradation. In yet another embodiment, the nucleic acid can be targeted in vivo for cell specific uptake and expression, by targeting a specific receptor (see, e.g., International Publication Nos. WO 92/06180; WO 92/22635; WO92/203 16; WO93/14188, WO 93/20221). Alternatively, the nucleic acid can be introduced intracellularly and incorporated within host cell DNA for expression, by homologous recombination (Koller and Smithies, 1989, PNAS 86:8932; and Zijlstra et al., 1989, Nature 342:435).

In a specific embodiment, viral vectors that contain the nucleic acid sequences of the invention are used. For example, a retroviral vector can be used (see Miller et al., 1993, Meth. Enzymol. 217:581). These retroviral vectors contain the components necessary for the correct packaging of the viral genome and integration into the host cell DNA. The nucleic acid sequences to be used in gene therapy are cloned into one or more vectors, which facilitates delivery of the nucleic acid into a subject. More detail about retroviral vectors can be found in Boesen et al., 1994, Biotherapy 6:291-302, which describes the use of a retroviral vector to deliver the mdr 1 gene to hematopoietic stem cells in order to make the stem cells more resistant to chemotherapy. Other references illustrating the use of retroviral vectors in gene therapy are: Clowes et al., 1994, J. Clin. Invest. 93:644-651; Klein et al., 1994, Blood 83:1467-1473; Salmons and Gunzberg, 1993, Human Gene Therapy 4:129-141; and Grossman and Wilson, 1993, Curr. Opin. in Genelics Devel. 3:110-114.

Adenoviruses are other viral vectors that can be used in gene therapy. Adenoviruses are especially attractive vehicles for delivering genes to respiratory epithelia. Adenoviruses naturally infect respiratory epithelia where they cause a mild disease. Adenoviruses have the advantage of being capable of infecting non-dividing cells. Kozarsky and Wilson, 1993, Current Opinion in Genetics Development 3:499 present a review of adenovirus-based gene therapy. Bout et al., 1994, Human Gene Therapy 5:3-10 demonstrated the use of adenovirus vectors to transfer genes to the respiratory epithelia of rhesus monkeys. Other instances of the use of adenoviruses in gene therapy can be found in Rosenfeld et al., 1991, Science 252:431; Rosenfeld et al., 1992, Cell 68:143; Mastrangeli et al., 1993, J. Clin. Invest. 91:225; International Publication No. WO94/12649; and Wang et al., 1995, Gene Therapy 2:775. In a preferred embodiment, adenovirus vectors are used. Adeno-associated virus (AAV) has also been proposed for use in gene therapy (Walsh et al., 1993, Proc. Soc. Exp. Biol. Med. 204:289-300; and U.S. Pat. No. 5,436,146).

Numerous techniques are known in the art for the introduction of foreign genes into cells (see, e.g., Loeffler and Behr, 1993, Meth. Enzymol. 217:599; Cohen et al., 1993, Meth. Enzymol. 217:618) and may be used in accordance with the present invention, provided that the necessary developmental and physiological functions of the recipient cells are not disrupted. The technique should provide for the stable transfer of the nucleic acid to the cell, so that the nucleic acid is expressible by the cell and preferably heritable and expressible by its cell progeny.

5.8 Prophylactic/Therapeutic Methods

The invention provides methods for treating, preventing, or managing a disorder in a subject/patient in whom the disorder is associated with the abnormal or undesirable activity of clk-2 of the invention. The subject/patient can be a mammal such as a non-primate (e.g., cows, pigs, horses, cats, dogs, rats, etc.) or a primate (e.g., monkey and a human). In a preferred embodiment, the subject is a human. By administrating to a subject a therapeutically or prophylactically effective amount of one or more agents of the invention, the activity of a polypeptide of the invention can be modulated to a desirable level.

Agents that can be used include but are not limited to: clk-2 proteins and analogs and derivatives (including fragments) thereof (e.g., as described hereinabove); antibodies thereto; nucleic acids encoding the clk-2 proteins, analogs, or derivatives; clk-2 antisense nucleic acids, and clk-2 agonists and antagonists. Disorders characterized by deregulated cellular growth, and particularly disorders in which decreased apoptosis is part of the pathology, e.g. cancers, are treated or prevented by administration of an agent that promotes clk-2. Such disorders can also be associated with cells showing an increase in telomere length. Disorders in which cellular regeneration or maintenance are deficient or desired, and particularly disorders in which the pathology depends completely or partially on apoptosis or increased apoptosis induced by oxidative stress are treated by administration of an agent that antagonizes (inhibits) clk-2 function. The above is described in detail in the subsections below.

As used herein, the term “prevent” refers to the prevention of the recurrence or onset of a disorder in a subject resulting from the administration of a prophylactic or therapeutic agent. As used herein, the term “manage” refers to the beneficial effects that a subject derives from a prophylactic or therapeutic agent, which does not result in a cure of the disorder. In certain embodiments, a subject is administered one or more prophylactic or therapeutic agents to “manage” a disorder so as to prevent the progression or worsening of the disorder.

As used herein, the term “prophylactic agent” refers to any agent that can be used in the prevention or prevention of the recurrence of a disorder. In certain embodiments, the term “prophylactic agent” refers to an agent of the invention. The term “prophylactic agent” can also refer to an agent used to prevent or prevent the recurrence of a disorder that is not based on the polypeptide of the invention. As used herein, a “prophylactically effective amount” refers to that amount of the prophylactic agent sufficient to result in the prevention of the recurrence of a disorder in a patient, including but not limited to, those predisposed to a disorder (e.g., those genetically predisposed) or those previously afflicted with the disorder. A prophylactically effective amount may also refer to the amount of the prophylactic agent that provides a prophylactic benefit in the prevention of a disorder. Further, a prophylactically effective amount with respect to a prophylactic agent of the invention means that amount of prophylactic agent alone, or in combination with other agents, that provides a prophylactic benefit in the prevention of a disorder. Used in connection with an amount of an agent of the invention, the term can encompass an amount that improves overall prophylaxis or enhances the prophylactic efficacy of or synergies with another prophylactic agent.

As used herein, the term “therapeutic agent” refers to any agent that can be used in the treatment or management of a disorder. The term “therapeutic agent” can also refer to an agent used in the treatment or management of a disorder that is not based on the polypeptide of the invention. As used herein, a “therapeutically effective amount” refers to that amount of the therapeutic agent sufficient to treat or manage a disorder, preferably, the amount sufficient to eliminate, modify, or control symptoms associated with such a disorder. A therapeutically effective amount may refer to the amount of therapeutic agent sufficient to delay or minimize the onset of the disorder. A therapeutically effective amount may also refer to the amount of the therapeutic agent that provides a therapeutic benefit in the treatment or management of a disorder. Further, a therapeutically effective amount with respect to a therapeutic agent of the invention means that amount of therapeutic agent alone, or in combination with other therapies, that provides a therapeutic benefit in the treatment or management of a disorder. Used in connection with an amount of an agent of the invention, the term can encompass an amount that improves overall therapy, reduces or avoids unwanted effects, or enhances the therapeutic efficacy of or synergies with another therapeutic agent.

The methods and compositions of the invention comprise the administration of one or more agents of the invention to subjects/patients suffering from or expected to suffer from a disorder associated the abnormal or undesirable activity of clk-2. Such abnormal or undesirable activity can be the result of a genetic predisposition, or exposure to environmental factors (e.g., a carcinogen) that increases the risk of developing the disorder. Such subjects may or may not have been previously treated for the disorder. The methods and compositions of the invention may be used as a first line or second line treatment. Thus, the subject may be undergoing other therapies that are not based on the polypeptides of the invention. The methods and compositions can be used in combination with another therapies. The methods and compositions of the invention can be used before any adverse effects or intolerance of these other therapies occurs.

Generally, administration of products of a species origin or species reactivity (in the case of antibodies) that is the same species as that of the patient is preferred. Thus, in a preferred embodiment, a human clk-2 protein, derivative, or analog, or nucleic acid, or an antibody to a human clk-2 protein, is therapeutically or prophylactically administered to a human patient.

Diseases and disorders characterized by deregulated cellular growth that are typically associated with decreased apoptosis or increased telomere length can be treated or prevented by administration of an agent that promotes (i.e., increases or supplies) clk-2 function. Preferably, the agents are targeted to cells that show decreased apoptosis, or cells in which apoptosis is to be promoted. Examples of such an agent include but are not limited to clk-2 proteins, derivatives, or fragments that are functionally active, particularly those that are active in cellular growth inhibition (e.g., as demonstrated in in vitro assays or in animal models), and nucleic acids encoding a clk-2 protein or functionally active derivative or fragment thereof (e.g., for use in gene therapy). Other agents that can be used, e.g., clk-2 agonists, can be identified using in vitro assays or animal models, examples of which are described infra.

In specific embodiments, agents that promote clk-2 function are administered therapeutically or prophylactically: (1) in diseases or disorders involving an absence or decreased (relative to normal or desired) level of clk-2 protein or function, for example, in patients where clk-2 protein is lacking, genetically defective, biologically inactive, underactive, or underexpressed; or (2) in diseases or disorders wherein in vitro (or in vivo) assays indicate the utility of clk-2 agonist administration e.g., by testing apoptosis functions. The absence or decreased level in clk-2 protein or function can be readily detected, e.g., by obtaining a patient tissue sample (e.g., from biopsy tissue) and assaying it in vitro for RNA or protein levels, structure and/or activity of the expressed clk-2 RNA or protein. Many methods standard in the art can be thus employed, including but not limited to phosphoprotein assays, immunoassays to detect and/or visualize clk-2 protein (e.g., western blot, immunoprecipitation followed by sodium dodecyl sulfate polyacrylamide gel electrophoresis, immunocytochemistry, etc.) and/or hybridization assays to detect clk-2 expression by detecting and/or visualizing clk-2 mRNA (e.g., northern assays, dot blots, in situ hybridization, etc.), etc.

Diseases and disorders involving deregulated cellular growth that can be treated or prevented include but are not limited to proliferative disorders, neoplastic growth, and cancers, etc. For a review of such disorders, see Fishman et al., 1985, Medicine, 2d Ed., J. B. Lippincott Co., Philadelphia. As used herein, “cancer” refers to primary or metastatic cancers. Examples of these are detailed below.

In particular embodiments, methods of the invention can be used to treat and/or prevent metastasis from primary tumors. Examples of such cancers include the following: leukemias (e.g., acute leukemia, acute lymphocytic leukemia, acute myelocytic leukemias, myeloblastic, promyelocytic, myelomonocytic, monocytic, erythroleukemia leukemia, myelodysplastic syndrome, chronic myelocytic/granulocytic leukemia, chronic lymphocytic leukemia, hairy cell leukemia, polycythemia vera); lymphomas (e.g., Hodgkin's disease, non-Hodgkin's disease); multiple myelomas (e.g., smoldering multiple myeloma, nonsecretory myeloma, osteosclerotic myeloma, plasma cell leukemia, solitary plasmacytoma and extramedullary plasmacytoma, Waldenstrom's macroglobulinemia, monoclonal gammopathy of undetermined significance, benign monoclonal gammopathy, heavy chain disease); bone and connective tissue sarcomas (e.g., bone sarcoma, osteosarcoma, osteogenic sarcoma, chondrosarcoma, Ewing's sarcoma, malignant giant cell tumor, fibrosarcoma of bone, chordoma, periosteal sarcoma, soft-tissue sarcomas, angiosarcoma (hemangiosarcoma), fibrosarcoma, Kaposi's sarcoma, leiomyosarcoma, liposarcoma, lymphangiosarcoma, neurilemmoma, rhabdomyosarcoma, synovial sarcoma); brain tumors (e.g., glioma, astrocytoma, brain stem glioma, ependymoma, oligodendroglioma, nonglial tumor, acoustic neurinoma, craniopharyngioma, medulloblastoma, meningioma, pineocytoma, pineoblastoma, primary brain lymphoma); breast cancer (e.g., adenocarcinoma, lobular/small cell carcinoma, intraductal carcinoma, medullary breast cancer, mucinous breast cancer, tubular breast cancer, papillary breast cancer, Paget's disease, inflammatory breast cancer); adrenal cancers (e.g., pheochromocytom and adrenocortical carcinoma); thyroid cancers (e.g., papillary or follicular thyroid cancer, medullary thyroid cancer and anaplastic thyroid cancer); pancreatic cancers (e.g., insulinoma, gastrinoma, glucagonoma, vipoma, somatostatin-secreting tumor, carcinoid or islet cell tumor); pituitary cancers (e.g., Cushing's disease, prolactin-secreting tumor, acromegaly, and diabetes insipius); eye cancers (e.g., ocular melanoma such as iris melanoma, choroidal melanoma, and cilliary body melanoma, retinoblastoma); vaginal cancers (e.g., squamous cell carcinoma, adenocarcinoma, and melanoma); vulvar cancer (e.g., squamous cell carcinoma, melanoma, adenocarcinoma, basal cell carcinoma, sarcoma, Paget's disease); cervical cancers (e.g., squamous cell carcinoma, adenocarcinoma); uterine cancers (e.g., endometrial carcinoma and uterine sarcoma); ovarian cancers (e.g., ovarian epithelial carcinoma, borderline tumor, germ cell tumor, and stromal tumor); esophageal cancers (e.g., squamous cancer, adenocarcinoma, adenoid cyctic carcinoma, mucoepidermoid carcinoma, adenosquamous carcinoma, sarcoma, melanoma, plasmacytoma, verrucous carcinoma, and oat cell/small cell carcinoma); stomach cancers (e.g., adenocarcinoma, fungating/polypoid, ulcerating, superficial spreading, diffusely spreading, malignant lymphoma, liposarcoma, fibrosarcoma, carcinosarcoma); colon cancers; rectal cancers; liver cancers (e.g., hepatocellular carcinoma, hepatoblastoma); gallbladder cancers (e.g., adenocarcinoma); cholangiocarcinomas (e.g., pappillary, nodular, diffuse); lung cancers (e.g., non-small cell lung cancer, squamous cell carcinoma, epidermoid carcinoma, adenocarcinoma, large-cell carcinoma and small-cell lung cancer, bronchogenic carcinoma); testicular cancers (e.g., germinal tumor, seminoma, anaplastic, spermatocytic, nonseminoma, embryonal carcinoma, teratoma carcinoma, choriocarcinoma/yolk-sac tumor); prostate cancers (e.g., adenocarcinoma, leiomyosarcoma, rhabdomyosarcoma); oral cancers (e.g., squamous cell carcinoma, basal cancers, salivary gland cancers, mucoepidermoid carcinoma, adenoidcystic carcinoma); pharynx cancers (e.g., squamous cell cancer, verrucous); skin cancers (e.g., basal cell carcinoma, squamous cell carcinoma, melanoma, superficial spreading melanoma, nodular melanoma, lentigo malignant melanoma, acral lentiginous melanoma, xeroderma pigmentosum, keratoactanthoma); kidney cancers (e.g., renal cell carcinoma, adenocarcinoma, hypernephroma, fibrosarcoma, transitional cell cancer, Wilms' tumor); bladder cancers (e.g., transitional cell carcinoma, squamous cell cancer, adenocarcinoma, carcinosarcoma); myxosarcoma; endotheliosarcoma; lymphangioendotheliosarcoma; mesothelioma; synovioma; hemangioblastoma; cystadenocarcinoma; sebaceous gland carcinoma.

In a preferred embodiment, cancers associated with or caused by aberrations in apoptosis are treated by the methods and compositions of the invention. Such cancers may include but not be limited to follicular lymphomas; carcinomas with p53 mutations; hormone dependent tumors of the breast, prostate and ovary; precancerous lesions such as familial adenomatous polyposis, and myelodysplastic syndromes. The absence of, decrease in or resistance to apoptosis in cancer cells or abnormal cells can readily be determined by techniques known in the art. Preferably, agents that promote clk-2 function are targeted to specific populations of cells within an organ or tissue that exhibit (i) an absence of or decreased level of clk-2 function, and/or (ii) an absence of or decreased level of apoptosis. The action of such agents can be augmented by administering one or more other modalities that increase oxidative stress which can in turn stimulate apoptosis.

The agents of the invention that promote clk-2 activity can also be administered to treat premalignant conditions and to prevent progression to a neoplastic or malignant state, including but not limited to those tumors and cancers listed above. Such prophylactic or therapeutic use is indicated in conditions known or suspected of preceding progression to neoplasia or cancer, in particular, where non-neoplastic cell growth consisting of hyperplasia, metaplasia, or most particularly, dysplasia has occurred (for review of such abnormal growth conditions, see Robbins and Angell, 1976, Basic Pathology, 2d Ed., W. B. Saunders Co., Philadelphia, pp. 68-79). Alternatively or in addition to the presence of abnormal cell growth characterized as hyperplasia, metaplasia, or dysplasia, the presence of one or more characteristics of a transformed phenotype, or of a malignant phenotype, displayed in vivo or displayed in vitro by a cell sample from a patient, can indicate the desirability of prophylactic/therapeutic administration of an agent that promotes clk-2 function. As mentioned supra, such characteristics of a transformed phenotype include morphology changes, looser substratum attachment, loss of contact inhibition, loss of anchorage dependence, protease release, increased sugar transport, decreased serum requirement, expression of fetal antigens, etc.

In another embodiment of the invention, an agent that promotes clk-2 activity is used to treat or prevent dysproliferative disorders. Specific embodiments are directed to treatment or prevention of cirrhosis of the liver (a condition in which scarring has overtaken normal liver regeneration processes) and defective wound healing (where epithelial cells fail to provide a barrier due to decreased proliferation).

In another embodiment, autoimmune disorders are treated, managed, or prevented by methods and compositions of the present invention. Inhibition or failure of the apoptotic cell death mechanism may contribute to diseases of the immune system by allowing persistence of self-reactive B and T lymphocyte cells, thereby promoting autoimmune disorders (see e.g., Watanabe-Fukunaga et al., 1992, Nature 356:314-317). One or more agents of the invention that increase apoptosis are administered either alone or in combination with a non-clk-2-based therapeutic agent to a subject in need thereof.

In yet another specific embodiment, rapid or inappropriate aging disorders are treated, managed, or prevented by methods and compositions of the present invention. One or more agents of the invention that increase telomere length are administered either alone or in combination with a non-clk-2-based therapeutic agent to a subject in need thereof.

In a second embodiment, degenerative disorders are treated, managed, or prevented by methods and compositions of the present invention. In a more specific embodiment, the degenerative disorders are associated with increased apoptosis, especially apoptosis triggered by oxidative stress. Diseases and disorders in which growth or regeneration are desired, e.g., neurodegenerative diseases, are treated by administration of an agent that antagonizes (inhibits) clk-2 function. The diseases or disorders which can be treated include, but are not limited to, degenerative nervous system diseases including but not limited to Alzheimer's disease, Parkinson's disease, Huntington's Chorea, and amyotrophic lateral sclerosis. Such diseases or disorders can be treated by administering compounds that interfere with clk-2 activity (e.g., a dominant negative clk-2 derivative; antibodies to clk-2; anti-sense nucleic acids that encode clk-2; clk-2 ribozymes or chemical groups that bind an active site of clk-2).

In specific embodiments, agents that inhibit clk-2 function are administered therapeutically and prophylactically: (1) in diseases or disorders involving an increased (relative to normal or desired) level of clk-2 protein or function, for example, in patients where clk-2 protein is overactive or overexpressed; or (2) in diseases or disorders wherein in vitro (or in vivo) assays indicate the utility of clk-2 antagonist administration, e.g., by testing apoptosis functions. The increased levels in clk-2 protein or function can be readily detected, e.g., by quantifying protein and/or RNA, by obtaining a patient tissue sample (e.g., from biopsy tissue) and assaying it in vitro for RNA or protein levels, structure and/or activity of the expressed clk-2 RNA or protein. Many methods standard in the art can be thus employed, including but not limited to kinase assays, immunoassays to detect and/or visualize clk-2 protein (e.g., western blot, immunoprecipitation followed by sodium dodecyl sulfate polyacrylamide gel electrophoresis, immunocytochemistry, etc.) and/or hybridization assays to detect clk-2 expression by detecting and/or visualizing respectively clk-2 mRNA (e.g., northern assays, dot blots, in situ hybridization, etc.), etc.

5.8.1 Determination of Therapeutic/Prophylactic Utility

The protocols and compositions of the invention are preferably tested in vitro, and then in vivo, for the desired therapeutic or prophylactic activity, prior to use in humans. For example, in vitro assays which can be used to determine whether administration of a specific therapeutic protocol is indicated, include in vitro cell culture assays in which a patient tissue sample is grown in culture, and exposed to or otherwise administered a protocol, and the effect of such protocol upon the tissue sample is observed. Alternatively, instead of culturing cells from a patient, agents and methods may be screened using cells of a relevant cell line (e.g. tumor or malignant cell line in the case of cancer).

Agents for use in therapy can be tested in suitable animal model systems prior to testing in humans, including but not limited to in rats, mice, chicken, cows, monkeys, rabbits, hamsters, etc. The agents can then be used in the appropriate clinical trials.

Toxicity and efficacy of the prophylactic and/or therapeutic protocols of the instant invention can be determined by standard pharmaceutical procedures in cell cultures or experimental animals, e.g., for determining the LD₅₀ (the dose lethal to 50% of the population) and the ED₅₀ (the dose therapeutically effective in 50% of the population). The dose ratio between toxic and therapeutic effects is the therapeutic index and it can be expressed as the ratio LD₅₀/ED₅₀. Prophylactic and/or therapeutic agents that exhibit large therapeutic indices are preferred. While prophylactic and/or therapeutic agents that exhibit toxic side effects may be used, care should be taken to design a delivery system that targets such agents to the site of affected tissue in order to minimize potential damage to uninfected cells and, thereby, reduce side effects.

The data obtained from the cell culture assays and animal studies can be used in formulating a range of dosage of the prophylactic and/or therapeutic agents for use in humans. The dosage of such agents lies preferably within a range of circulating concentrations that include the ED₅₀ with little or no toxicity. The dosage may vary within this range depending upon the dosage form employed and the route of administration utilized. For any agent used in the method of the invention, the therapeutically effective dose can be estimated initially from cell culture assays. A dose may be formulated in animal models to achieve a circulating plasma concentration range that includes the IC₅₀ (i.e., the concentration of the test compound that achieves a half-maximal inhibition of symptoms) as determined in cell culture. Such information can be used to more accurately determine useful doses in humans. Levels in plasma may be measured, for example, by high performance liquid chromatography.

For example, for agents used to treat, manage, or prevent cancer, the anti-cancer activity of the therapies used in accordance with the present invention also can be determined by using various experimental animal models for the study of cancer such as the SCID mouse model or transgenic mice where a mouse clk-2 is replaced with the human clk-2, nude mice with human xenografts, animal models, such as hamsters, rabbits, etc. known in the art and described in Relevance of Tumor Models for Anticancer Drug Development (1999, eds. Fiebig and Burger); Contributions to Oncology (1999, Karger); The Nude Mouse in Oncology Research (1991, eds. Boven and Winograd); and Anticancer Drug Development Guide (1997 ed. Teicher).

5.8.2 Agent Targeting

The present invention encompasses the use of agents of the invention that are recombinantly fused or chemically conjugated (including both covalent and non-covalent conjugations) to a targeting moiety such as, but not limited to, antibodies or fragments thereof. For example, the agents of the invention may be fused or conjugated to a chimeric antibody, humanized antibody, Fab fragment, Fd fragment, Fv fragment, F(ab)₂ fragment, or portion thereof. Conjugated antibodies can be used to target agents of the invention to particular cell types associated with the disorder to be treated. Such targeting can improve the efficacy by increasing the concentration of targeted agent at the desired site. Also, toxicity or side effects of treatment can be minimized by reducing systemic exposure to the agent.

A conjugated agent's relative efficacy in comparison to the free agent can depend on a number of factors. For example, rate of uptake of the antibody-agent into the cell (e.g, by endocytosis), rate/efficiency of release of the agent from the antibody, rate of export of the agent from the cell, etc. can all effect the action of the agent. Antibodies used for targeted delivery of agents can be assayed for the ability to be endocytosed by the relevant cell type (i.e., the cell type associated with the disorder to be treated) by any method known in the art. Additionally, the type of linkage used to conjugate an agent to an antibody should be assayed by any method known in the art such that the agent action within the target cell is not impeded.

In another embodiment, antibodies can be fused or conjugated to liposomes, wherein the liposomes are used to encapsulate agents of the invention (see e.g., Park et al., 1997, Can. Lett. 118:153-160; Lopes de Menezes et al., 1998, Can. Res. 58:3320-30; Tseng et al., 1999, Int. J. Can. 80:723-30; Crosasso et al., 1997, J. Pharm. Sci. 86:832-9). In a preferred embodiment, the pharmokinetics and clearance of liposomes are improved by incorporating lipid derivatives of PEG into liposome formulations (see e.g., Allen et al., 1991, Biochem Biophys Acta 1068:133-41; Huwyler et al., 1997, J. Pharmacol. Exp. Ther. 282:1541-6).

In one embodiment, the disorder to be treated is a disorder associated with increased apoptosis, especially apoptosis due to oxidative stress. Agents of the invention that decrease clk-2 polypeptide activity or clk-2 gene expression can be conjugated to antibodies and targeted to the affected cell types. In a specific embodiment, the targeted cell type is a neuron and the disorder is a neurodegenerative disorder, especially Parkinson's Disease, Alzheimer's Disease, Huntington's Chorea, and amyotrophic lateral sclerosis. In another specific embodiment, the target cell type is a liver cell and the disorder is liver cirrhosis. In another specific embodiment, the target cell type is a kidney cell and the disorder is polycystic kidney disease.

In another embodiment, the disorder to be treated is a disorder associated with decreased telomere length. Agents of the invention that increase clk-2 polypeptide activity or clk-2 gene expression can be conjugated to antibodies and targeted to the affected cell types. In a specific embodiment, the disorder is rapid aging. In another specific embodiment, the target cell type is a liver cell and the disorder is liver cirrhosis. In another specific embodiment, the target cell type is an epithelial cell and the disorder is decreased wound healing.

In another embodiment, the disorder to be treated is a disorder associated with increased telomere length. Agents of the invention that decrease clk-2 polypeptide activity or clk-2 gene expression can be conjugated to antibodies and targeted to the affected cell types. In a specific embodiment, the targeted cell type is a cancer cell including metastasis, especially colorectal cancer, breast cancer, or skin cancer.

In another embodiment, the disorder to be treated is a disorder associated with decreased apoptosis. Agents of the invention that increase clk-2 polypeptide activity or elk-2 gene expression can be conjugated to antibodies and targeted to the affected cell types. In a specific embodiment, the targeted cell type is an autoimmune lymphocyte. In a specific embodiment, the targeted cell type is a cancer cell including metastasis, especially colorectal cancer, breast cancer, or skin cancer.

In a specific embodiment, the disorder to be treated is cancer. Agents of the invention that increase apoptosis, decrease telomere length, or slow/stop cell cycle progression can be fused or conjugated to an antibody that specifically targets to tumor cells. Such conjugated agents will be targeted to the desired site of action (see e.g., Trail & Bianchi, 1999, Curr. Opin. Immunol. 11:584-88; Panchal, 1998, Biochem. Pharmacol. 55:247-52). Examples of such monoclonal antibodies that immunospecifically bind tumor-associated antigens expressed at a higher density on malignant cells relative to normal cells can be found in the art, e.g., listed in Table 2.

TABLE 2

| cancer | antibody | antigen | reference |
|---|---|---|---|
| colorectal | MAb 17-1A | epithelial | Kufer et al, 1997, |
| | | | Cancer Immunol |
| | | | Immunother 45:193 |
| breast | antiHER2 MAb | HER2 | Pegram et al., 1998, |
| | | | J. Clin. Oncol. |
| | | | 16:2659 |
| breast | MAb CT-M-01 | polyepithelial | Hinman et al., 1993, |
| | | mucin | Can. Res. 53:3336 |
| carcinomas of | BR96 | Le^{y}-related | Trail et al., 1993, |
| lung, breast, | | tumor antigen | Science 261:212; |
| colon | | | Trail et al., 1995, |
| | | | Drug Dev Res |
| | | | 34:196 |
| carcinomas of | B3 | Le^{y}-related | Pai et al., 1996, |
| lung, breast, | | tumor antigen | Nat. Med. 2:350 |
| colon | | | |
| myeloid leukemia | CMA-676 | CD33 | Sievers et al., 1999, |
| | | | Blood 93:3678 |
| liver metastases | 14G2a | gangliside-GD₂ | Lode et al., 1998, |
| | | | Can Res 58:2925 |
| B-cell lymphoma | SIL | CD19 | Lopes de Menezes |
| | | | et al., 1998, Can. |
| | | | Res. 58:3320 |
| ovarian | 260F9 | | Pirker et al., 1985, |
| | | | J Clin Invest |
| | | | 76:1261 |
| ovarian | 454C11 | | Pirker et al., 1985, |
| | | | J Clin Invest |
| | | | 76:1261 |

In another specific embodiment, the disorder to be treated is an autoimmune disorder. Agents of the invention that increase apoptosis can be fused or conjugated to an antibody that specifically targets to autoimmune lymphocytes. Such conjugated agents will be targeted to the desired site of action. One of such monoclonal antibodies that immunospecifically binds autoimmune lymphocytes is an anti-idiotypic antibody. Such antibody recognizes as its antigen the antigen-binding portion of another antibody. An autoimmune antibody can be isolated and an anti-idiotypic antibody can be made that is specific for that particular autoimmune antibody using methods known in the art. Because antibody secreting cells (i.e., B cell lymphocytes) also express transmembrane forms of antibody, the anti-idiotypic antibody will target to cells secreting the autoimmune antibody.

Agents can be conjugated to antibodies by any method known in the art, including, but not limited to aldehyde/Schiff linkage, sulphydryl linkage, acid-labile linkage, cis-aconityl linkage, hydrazone linkage, enzymatically degradible linkage (see generally Garnett, 2002, Adv. Drug Deliv. Rev. 53:171-216). Additional techniques for conjugating therapeutic moieties to antibodies are well known, see, e.g., Arnon et al., “Monoclonal Antibodies For Immunotargeting Of Drugs In Cancer Therapy”, in Monoclonal Antibodies And Cancer Therapy, Reisfeld et al. (eds.), pp. 243-56 (Alan R. Liss, Inc. 1985); Hellstrom et al., “Antibodies For Drug Delivery”, in Controlled Drug Delivery (2nd Ed.), Robinson et al. (eds.), pp. 623-53 (Marcel Dekker, Inc. 1987); Thorpe, “Antibody Carriers Of Cytotoxic Agents In Cancer Therapy: A Review”, in Monoclonal Antibodies '84: Biological And Clinical Applications, Pinchera et al. (eds.), pp. 475-506 (1985), “Analysis, Results, And Future Prospective Of The Therapeutic Use Of Radiolabeled Antibody In Cancer Therapy”, in Monoclonal Antibodies For Cancer Detection And Therapy, Baldwin et al. (eds.), pp. 303-16 (Academic Press 1985), and Thorpe et al., 1982, Immunol. Rev. 62:119-58. Methods for fusing or conjugating antibodies to polypeptide agents are known in the art. See, e.g., U.S. Pat. Nos. 5,336,603, 5,622,929, 5,359,046, 5,349,053, 5,447,851, and 5,112,946; EP 307,434; EP 367,166; International Publication Nos. WO 96/04388 and WO 91/06570; Ashkenazi et al., 1991, PNAS 88: 10535-10539; Zheng et al., 1995, J. Immunol. 154:5590-5600; and Vil et al., 1992, PNAS 89:11337-11341. The fusion of an antibody to a agent does not necessarily need to be direct, but may occur through linker sequences. Such linker molecules are commonly known in the art and described in Denardo et al., 1998, Clin Cancer Res. 4:2483-90; Peterson et al., 1999, Bioconjug. Chem. 10:553; Zimmerman et al., 1999, Nucl. Med. Biol. 26:943-50; Garnett, 2002, Adv. Drug Deliv. Rev. 53:171-216.

In other embodiments, antibody properties can be altered as desired (e.g., antibodies or fragments thereof with higher affinities and lower dissociation rates) through the techniques of gene-shuffling, motif-shuffling, exon-shuffling, and/or codon-shuffling (collectively referred to as “DNA shuffling”). See, generally, U.S. Pat. Nos. 5,605,793; 5,811,238; 5,830,721; 5,834,252; and 5,837,458, and Patten et al., 1997, Curr. Opinion Biotechnol. 8:724-33; Harayama, 1998, Trends Biotechnol. 16:76; Hansson, et al., 1999, J. Mol. Biol. 287:265; and Lorenzo and Blasco, 1998, BioTechniques 24:308. Antibodies or fragments thereof, or the encoded antibodies or fragments thereof, may be altered by being subjected to random mutagenesis by error-prone PCR, random nucleotide insertion or other methods prior to recombination. One or more portions of a polynucleotide encoding an antibody or antibody fragment, which portions immunospecifically bind to an antigen expressed on a cell associated with a particular disorder may be recombined with one or more components, motifs, sections, parts, domains, fragments, etc. of one or more heterologous molecules.

In other embodiments, the antibodies or fragments thereof can be fused to marker sequences, such as a peptide, to facilitate purification. In preferred embodiments, the marker amino acid sequence is a hexa-histidine peptide, such as the tag provided in a pQE vector (QIAGEN, Inc., Chatsworth, Calif.,), among others, many of which are commercially available (see e.g., Gentz et al., 1989, PNAS 86:821) Other peptide tags useful for purification include, but are not limited to, the hemagglutinin (HA) tag, which corresponds to an epitope derived from the influenza hemagglutinin protein (Wilson et al., 1984, Cell 37:767) and the “flag” tag. Any purification method known in the art can be used (see e.g., International Publication WO 93/21232; EP 439,095; Naramura et al., 1994, Immunol. Lett. 39:91-99; U.S. Pat. No. 5,474,981; Gillies et al., 1992, PNAS 89:1428-1432; and Fell et al., 1991, J. Immunol. 146:2446-2452).

5.8.3 Combination Therapy with Other Prophylactic/Therapeutic Agents

In some embodiments, the invention provides methods for treating a disorder by administering one or more agents of the invention in combination with a prophylactic/therapeutic agent not based on clk-2. The present invention also relates to a method for increasing a patient's sensitivity to a therapeutic modality, comprising administering to a subject who is receiving, had received or will receive the therapeutic modality, a clk-2 agent of the invention (e.g., clk-2 nucleic acid, clk-2 polypeptide, clk-2 agonist, clk-2 antagonist, inhibitor of a clk-2 agonist, inhibitor of a clk-2 antagonist).

In one embodiment, the disorder is cancer and a clk-2 agent of the invention that increases apoptosis is administered in combination with a non-clk-2-based therapeutic. In a more specific embodiment, the clk-2 agent of the invention is administered to cancer patient to increase the tumor's susceptibility to apoptosis, which increases the tumor's sensitivity to subsequent challenge with a non-clk-2-based cancer therapeutic agent, especially one that causes apoptosis or oxidative stress. In another preferred embodiment, the non-clk-2 based therapeutic is a compound that slows or stops the cell cycle, such as those that target microtubule functions.

As used herein, the term “in combination” refers to the use of more than one prophylactic and/or therapeutic agents. The use of the term “in combination” does not restrict the order in which prophylactic and/or therapeutic agents are administered to a subject with a disorder. A first prophylactic or therapeutic agent can be administered prior to (e.g., 1 minute, 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks before), concomitantly with, or subsequent to (e.g., 1 minute, 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks) the administration of a second prophylactic or therapeutic agent to a subject which had, has, or is susceptible to a disorder. Any additional prophylactic or therapeutic agent can be administered in any order with the other additional prophylactic or therapeutic agents. In certain embodiments, an agent of the invention is one of the prophylactic and/or therapeutic agents administered. In certain embodiments, agent of the invention is administered in combination with a prophylactic and/or therapeutic agents that is not based on a polypeptide of the invention.

5.9 Pharmaceutical Compositions

The compositions of the invention include bulk drug compositions useful in the manufacture of pharmaceutical compositions (e.g., impure or non-sterile compositions) and parenteral pharmaceutical compositions (i.e., compositions that are suitable for administration to a subject or patient) which can be used in the preparation of unit dosage forms. Such compositions comprise a prophylactically or therapeutically effective amount of a prophylactic and/or therapeutic agent disclosed herein or a combination of those agents and a pharmaceutically acceptable carrier. Preferably, compositions of the invention comprise a prophylactically or therapeutically effective amount of one or more agents of the invention and a pharmaceutically acceptable carrier. In a further embodiment, the composition of the invention further comprises an additional prophylactic or therapeutic useful for treating, managing, or preventing the same disorder as the agent of the invention.

In a specific embodiment, the term “pharmaceutically acceptable” means approved by a regulatory agency of the Federal or a state government or listed in the U.S. Pharmacopeia or other generally recognized pharmacopeia for use in animals, and more particularly in humans. The term “carrier” refers to a diluent, adjuvant (e.g., Freund's adjuvant (complete and incomplete), excipient, or vehicle with which the therapeutic is administered. Such pharmaceutical carriers can be sterile liquids, such as water and oils, including those of petroleum, animal, vegetable or synthetic origin, such as peanut oil, soybean oil, mineral oil, sesame oil and the like. Water is a preferred carrier when the pharmaceutical composition is administered intravenously. Saline solutions and aqueous dextrose and glycerol solutions can also be employed as liquid carriers, particularly for injectable solutions. Suitable pharmaceutical excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. The composition, if desired, can also contain minor amounts of wetting or emulsifying agents, or pH buffering agents. These compositions can take the form of solutions, suspensions, emulsion, tablets, pills, capsules, powders, sustained-release formulations and the like.

Generally, the ingredients of compositions of the invention are supplied either separately or mixed together in unit dosage form, for example, as a dry lyophilized powder or water free concentrate in a hermetically sealed container such as an ampoule or sachette indicating the quantity of active agent. Where the composition is to be administered by infusion, it can be dispensed with an infusion bottle containing sterile pharmaceutical grade water or saline. Where the composition is administered by injection, an ampoule of sterile water for injection or saline can be provided so that the ingredients may be mixed prior to administration.

The compositions of the invention can be formulated as neutral or salt forms. Pharmaceutically acceptable salts include those formed with anions such as those derived from hydrochloric, phosphoric, acetic, oxalic, tartaric acids, etc., and those formed with cations such as those derived from sodium, potassium, ammonium, calcium, ferric hydroxides, isopropylamine, triethylamine, 2-ethylamino ethanol, histidine, procaine, etc.

5.9.1 Modes of Administration

Pharmaceutical compositions for use in accordance with the present invention may be formulated in conventional manner using one or more physiologically acceptable carriers or excipients. The formulation should suit the mode of administration. Various delivery systems are known and can be used to administer an agent of the invention or the combination of an agent of the invention and a prophylactic or therapeutic useful for treating, managing, or preventing the same disorder as the agent of the invention. Administration of the pharmaceutical compositions of the invention includes, but is not limited to, oral, inhalation, parenteral, intravenous, intramuscular, intraperitoneal, intraorbital, intraocular, intracapsular, intraspinal, intrastemal, intra-arterial, intradermal, subcutaneous, topical, depo injection, implantation, time-release mode, intracavitary, intranasal, intratumor, and controlled release, transmucosal, and rectal administration. The skilled artisan can appreciate the specific advantages and disadvantages to be considered in choosing a mode of administration.

Multiple modes of administration are encompassed by the invention. For example, a agent of the invention is administered by subcutaneous injection, whereas a combination therapeutic agent is administered by intravenous infusion.

Systemic administration can also be by transmucosal or transdermal means. For transmucosal or transdermal administration, penetrants appropriate to the barrier to be permeated are used in the formulation. Penetrants for transmucosal administration are generally known in the art, and include, for example, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art. Pharmaceutical compositions adapted for transdermal administration can be provided as discrete patches intended to remain in intimate contact with the epidermis for a prolonged period of time.

Pharmaceutical compositions adapted for topical administration to the eye include, for example, eye drops or injectable compositions. In these compositions, the active ingredient can be dissolved or suspended in a suitable carrier, which includes, for example, an aqueous solvent with or without carboxymethylcellulose. Pharmaceutical compositions adapted for topical administration in the mouth include, for example, lozenges, pastilles and mouthwashes.

Pharmaceutical compositions adapted for oral administration may be provided, for example, as capsules, tablets, powders, granules, solutions, syrups, suspensions (in aqueous or non-aqueous liquids), edible foams, whips, or emulsions. Tablets or hard gelatine capsules may comprise, for example, lactose, starch or derivatives thereof, magnesium stearate, sodium saccharine, cellulose, magnesium carbonate, stearic acid or salts thereof. Soft gelatin capsules may comprise, for example, vegetable oils, waxes, fats, semi-solid, or liquid polyols. Solutions and syrups may comprise, for example, water, polyols and sugars.

Pharmaceutically compatible binding agents, and/or adjuvant materials can be included as part of the composition. The tablets, pills, capsules, and troches can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

An active agent intended for oral administration may be coated with or admixed with a material (e.g., glyceryl monostearate or glyceryl distearate) that delays disintegration or affects absorption of the active agent in the gastrointestinal tract. Thus, for example, the sustained release of an active agent may be achieved over many hours and, if necessary, the active agent can be protected from being degraded within the gastrointestinal tract. Taking advantage of the various pH and enzymatic conditions along the gastrointestinal tract, pharmaceutical compositions for oral administration may be formulated to facilitate release of an active agent at a particular gastrointestinal location. Oral formulations preferably comprise 10% to 95% active ingredient by weight.

Pharmaceutical compositions adapted for nasal administration can comprise solid carriers such as powders (preferably having a particle size in the range of 20 to 500 microns). Powders can be administered in the manner in which snuff is taken, i.e., by rapid inhalation through the nose from a container of powder held close to the nose. Alternatively, compositions adopted for nasal administration may comprise liquid carriers such as, for example, nasal sprays or nasal drops. These compositions may comprise aqueous or oil solutions of the active ingredient. Compositions for administration by inhalation may be supplied in specially adapted devices including, but not limited to, pressurized aerosols, nebulizers, or insufflators, which can be constructed so as to provide predetermined dosages of the active ingredient.

Pharmaceutical compositions adapted for rectal administration can be prepared in the form of suppositories (e.g., with conventional suppository bases such as cocoa butter and other glycerides) or retention enemas for rectal delivery. Pharmaceutical compositions adapted for vaginal administration may be provided, for example, as pessaries, tampons, creams, gels, pastes, foams, or spray formulations.

In one embodiment, a pharmaceutical composition of the invention is delivered by a controlled-release system. Controlled release systems are discussed in the review by Langer (1990, Science 249:1527-1533). Any technique known to one of skill in the art can be used to produce sustained release formulations comprising one or more therapeutic agents of the invention. See, e.g., U.S. Pat. No. 4,526,938; International Publication Nos. WO 91/05548 and WO 96/20698; Ning et al., 1996, Radiotherapy & Oncology 39:179-189; Song et al., 1995, PDA Journal of Pharmaceutical Science & Technology 50:372-397; Cleek et al., 1997, Pro. Int'l. Symp. Control. Rel. Bioact. Mater. 24:853-854; and Lam et al., 1997, Proc. Int'l. Symp. Control Rel. Bioact. Mater. 24:759-760. For example, the pharmaceutical composition may be administered using intravenous infusion, an implantable osmotic pump, a transdermal patch, liposomes, or other modes of administration. In one embodiment, a pump may be used (See, e.g., Langer, 1990, Science 249:1527-33; Sefton, 1987, CRC Crit. Ref Biomed. Eng. 14:201; Buchwald et al., 1980, Surgery 88:507; Saudek et al., 1989, N. Eng. J. Med. 321:574). In another embodiment, the compound can be delivered in a vesicle, in particular a liposome (See, e.g., Langer, 1990, Science 249:1527-1533; Treat et al., 1989, in Liposomes in the Therapy of Infectious Disease and Cancer, Lopez-Berestein and Fidler (eds.) Liss, New York, pp. 353-65; Lopez-Berestein, ibid., pp. 317-27; International Patent Publication No. WO 91/04014; U.S. Pat. No. 4,704,355). In another embodiment, polymeric materials can be used (See, e.g., Medical Applications of Controlled Release, Langer and Wise (eds.) CRC Press: Boca Raton, Fla., 1974; Controlled Drug Bioavailability, Drug Product Design and Performance, Smolen and Ball (eds.) Wiley: New York (1984); Ranger and Peppas, 1953, J. Macromol. Sci. Rev. Macromol. Chem. 23:61; Levy et al., 1985, Science 228:190; During et al., 1989, Ann Neurol. 25:351; Howard et al., 1989, J. Neurosurg. 71:105).

In one embodiment, the active compounds, which comprise polynucleotides, polypeptides, antibodies, or other agents of the invention, are prepared with carriers that will protect the compound from rapid elimination from the body. Such carriers can be a controlled release formulation, which includes, but is not limited to, implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc. Liposomal suspensions (including liposomes targeted to infected cells with monoclonal antibodies to viral antigens) can also be used as pharmaceutically acceptable carriers. These can be prepared according to methods known to those skilled in the art, for example, as described in U.S. Pat. No. 4,522,811.

In a particular embodiment, polypeptides of the invention can be administered using a biodegradable polymer having reverse thermal gelatin properties (See, e.g., U.S. Pat. No. 5,702,717).

In yet another embodiment, a controlled release system can be placed in proximity of the target. For example, to treat cancer, a micropump may deliver controlled doses directly into the tumor region, thereby requiring only a fraction of the systemic dose (See, e.g., Goodson, 1984, in Medical Applications of Controlled Release, vol. 2, pp. 115-138).

In one embodiment, it may be desirable to administer a pharmaceutical composition of the invention locally to the area in need of treatment; this may be achieved, for example, by local infusion during surgery, topical application (e.g., in conjunction with a wound dressing after surgery), injection, by means of a catheter, by means of a suppository, or by means of an implant. An implant can be of a porous, non-porous, or gelatinous material, including membranes, such as sialastic membranes, or fibers.

Pharmaceutical compositions suitable for injectable use include sterile aqueous solutions, or dispersions, or sterile powders (for the extemporaneous preparation of sterile injectable solutions or dispersions). For intravenous administration, suitable carriers include physiological saline, bacteriostatic water, Cremophor ELTM (BASF; Parsippany, N.J.) or phosphate buffered saline (PBS). The carrier can be a solvent or dispersion medium comprising, for example, water, ethanol, polyol (for example, glycerol, propylene glycol, and liquid polyetheylene glycol, and the like), and suitable mixtures thereof. The proper fluidity can be maintained, for example, by the use of a coating such as lecithin, by the maintenance of the required particle size in the case of dispersion, or by the use of a surfactant. Prevention of the action of microorganisms can be achieved by various antibacterial and antifungal agents, such as for example, parabens, chlorobutanol, phenol, ascorbic acid, and thimerosal. It can be preferable to include in the composition isotonic agents, such as for example, sugars, polyalcohols (e.g., mannitol), sorbitol, and sodium chloride. Prolonged absorption of the injectable compositions can be brought about by including in the composition an agent which delays absorption, such as for example, aluminum monostearate and gelatin.

For administration by inhalation, the compounds are delivered in the form of an aerosol spray from a pressurized container or dispenser which comprises a suitable propellant, e.g., a gas such as carbon dioxide, or a nebulizer.

Oral or parenteral compositions can be formulated in dosage unit form for ease of administration and uniformity of dosage. Dosage unit form as used herein refers to physically discrete units suited as unitary dosages for the subject to be treated, such that each unit contains a predetermined quantity of active compound, which is calculated to produce the desired therapeutic effect, and a pharmaceutical carrier. The skilled artisan will appreciate that dosage unit forms are dependent on the unique characteristics of the active compound, the particular therapeutic effect to be achieved, and the limitations inherent in the art of compounding such an active compound for human administration.

The skilled artisan will appreciate that certain factors may influence the dose necessary to effectively treat a subject, which factors include, but are not limited to, previous treatment regimens, severity of the disorder, general health and/or age of the subject, and concurrent disorders. Moreover, treatment of a subject with a therapeutically effective amount of an agent of the invention can include a single treatment or, preferably, can include a series of treatments.

5.10 Kits

The invention also encompasses kits for detecting the presence and/or amount of a polypeptide or nucleic acid of the invention in a biological sample (a test sample). Such kits can be used to determine if a subject is suffering from or is at increased risk of developing a disorder associated with aberrant expression of a polypeptide or nucleic acid of the invention. The kits of the invention may comprise additionally instructions on the use of the components in the kit.

In an exemplary embodiment, a kit comprises one or more agents that bind to the nucleic acids of the invention (e.g., complementary nucleic acid fragment or probe) or polypeptide of the invention (e.g. antibody). For antibody-based kits, the kit can comprise, for example: (1) a first antibody (e.g., attached to a solid support) which binds to a polypeptide of the invention; and, optionally, (2) a second, different antibody which binds to the first antibody and is conjugated to a detectable agent. For oligonucleotide-based kits, the kit can comprise, for example: (1) an oligonucleotide, e.g., a detectably labeled oligonucleotide, which hybridizes to a nucleic acid encoding a polypeptide of the invention or (2) a pair of primers useful for amplifying a nucleic acid encoding a polypeptide of the invention. In a specific embodiment, clk-2 agents are included in the kit. Such kits can be used to indicate those subjects that are suffering from or have an increased risk of developing disorders such as cancer, neurodegenerative disorders, and autoimmune disorders.

The invention also provides a kit comprising one or more containers filled with a prophylactic/therapeutic agent of the invention. Additionally, one or more other prophylactic/therapeutic agents useful for the treatment of a disorder can also be included in the kit. In one embodiment, the kit comprises one or more clk-2 agents of the invention and is useful for treating, preventing, or managing a disorder associated with aberrant or undesirable clk-2 expression/activity.

### 6. EXAMPLES

The following examples demonstrate the phenotypes of clk-2 mutations, and the cloning and characterization of a clk-2 nucleic acid molecule from C. elegans. The exemplary C. elegans mutants described herein and other similar mutants can be used in various screening assays for identifying compounds that may interact with clk-2. The compounds identified in such assays can be further tested in assays using mammalian cells and human clk-2.

6.1 Materials and Methods

6.1.1 Nematode Strains and Genetic Analysis

Growth conditions were as described (Brenner, 1974, Genetics, 77:71-94), at 20° C. unless specified otherwise. Strains and/or alleles used: N2 (wild type), MQ125 clk-2(qm37) outcrossed 12 times, lin-13(n387), lin-39(n1760), mab-5(e1239), sma-3(e491), UBC-32(el89), UBC-36(e251), glp-4(bn2ts), fem-2(b245ts) and fem-3(q20ts). Genotypes used and scored for genetic mapping have been submitted to WormBase.

Developmental and behavioral phenotypes at 20° C. were scored as described (Wong et al., 1995, Genetics 139:1247-59). For temperature shifts experiments between 20 and 25° C., embryos or worms were transferred onto preincubated plates. Two- to four-cell stage embryos were dissected as described (Wong et al., 1995).

6.1.2 Plasmids and Transgenic Strains

Cosmid C₀₇H6 (Accession Number, AC006605). pMQ248 is a transcriptional fusion of the promoter region of the clk-2 operon (bases 37319 to 36932 of C₀₇H6), including only 23 bp of cux-7 sequence, to GFP in pPD95.77. pMQ254 is a similar transcriptional fusion, with a larger promoter region (bases 40010 to 36932 of C₀₇H6). pMQ251 is a translational fusion of clk-2 to GFP of vector pPD95.77. This fusion construct includes, in addition to the promoter region described for pMQ248 (i.e., bases 37319 to 36932), the entire coding region of clk-2 (bases 37319-36932) excluding its stop codon and only a small part of the coding region of cux-7 (bases 36955-36546, and 35077-34999, where bases 36545-35078 have been deleted). Also, the 3′ UTR of UBC-54 present in vector pPD95.77 has been replaced by the 3′ UTR of clk-2 (bases 31654-30501 of C₀₇H6). pMQ251 rescues the clk-2 phenotypes, including development and behavior at 20° C., and lethality at 25° C. The three constructs were microinjected into wild-type and clk-2(qm37) worms at a concentration of 100 ng/μl along with pRF4 125 ng/μl˜100 F₁ transgenic worms were examined, and ˜30 F₂ transgenics for each of approx. seven independent lines obtained with each construct, by epifluorescence. In addition, wild-type and clk-2(qm37) worms were microinjected with blunt linear fragments of these constructs and pRF4, at of 1 ng/μl, along with 100 ng/μl of PvuII-N2 genomic DNA (complex arrays). Approx. 50 F₁ transgenic worms were examined, and ˜30 F₂ transgenics for each of ˜10 independent lines obtained with each construct. A high level of expression is detected in all somatic tissues of F₁ animals, but by the F₂ generation, most transgenic worms express clk-2::GFP in the same tissues at very weak levels.

MQ691 clk-2(qm37); qmEx159 was generated by microinjection of pMQ246 at 50 ng/μl, pRF4 at 125 ng/μl, and salmon sperm DNA at 100 ng. pMQ246 contains bases 37319 to 31528 of C₀₇H6, excluding bases 36544 to 35077, cloned in pBluescript, thus comprising the promoter region of the clk-2 operon, part of cux-7 with a large internal deletion that interrupts its reading frame and the entire coding sequence of clk-2. A similar clone containing the qm37 mutation fails to rescue the clk-2 phenotypes.

6.1.3 Mutagenesis and Screening for clk-2(qm37) Suppressors

clk-2(qm37) mutants grown at 20° C. were mutagenized with 25 mM ethyl methane sulfonate (EMS) following the standard protocol (Sulston & Hodgkin, 1988, pp. 587-606, in The Nematode Caenorhabditis elegans, Wood, Ed., Cold Spring Harbor Laboratory Press, Cold Spring Harbor, N.Y.). Mutagenized worms were washed with M9 and spotted onto seeded plates. One to two hours later, groups of 5 young adults (P0s) were plated onto new 9 cm petri dishes and allowed to self-fertilize at 20° C. The P0s were removed after they had laid ˜150 eggs (30 eggs each). The number of F1 worms was counted on a number of plates to estimate the number of haploid genomes screened at each round. The F1s were allowed to develop at 20° C. until a majority had reached the L2-L3 stages. The plates were then shifted to 25° C. and the F1s completed larval development at 25° C. The F2 broods were examined 3-4 days later. Most plates contained scrawny and sterile F1 worms that produced few dead F2 embryos. Rare plates carried fertile worms that produced live F2 progeny. These plates were considered to carry clk-2(qm37) suppressors and were kept for further analysis.

In another protocol, the F1s were allowed to develop and self-fertilize at 20° C. until a majority of the F2s had reached the L2-L3 stages. The plates were then shifted to 25° C. and the F2s completed larval development at 25° C. The F3 broods were examined 3-4 days later. Most plates contained scrawny and sterile F2 worms that produced few dead F3 embryos. Rare plates carried fertile worms that produced live F3 progeny. These plates were considered to carry clk-2(qm37) suppressors and were kept for further analysis.

6.1.4 RT-PCR

RT-PCR experiments were performed from mixed stage N2 total RNA essentially as described (Ewbank et al., 1997, Science 275:980-3). No product could be amplified using primer pairs corresponding to the 3′ region of cux-7 and the 5′ region of clk-2, supporting that the operon encodes two genes that result in separate mRNAs.

6.1.5 RNA Interference

Transcription with T3 and T7 polymerases was performed on gel purified linearized clones yk447b4 (clk-2 cDNA) and yk215f6 (cux-7 cDNA) using RNA synthesis kit (Promega). Single-stranded RNAs were annealed and injected as described (Fire et al., 1998).

6.1.6 Northern Analysis

Worm populations were synchronized at different developmental stages as described by Wood (Wood, 1988). 10 μg of total RNA (Trizol extraction, Gibco) or 0.5 μg of polyA+ selected RNA (Qiagen) were fractionated by electrophoresis in denaturing conditions, transferred to Hybond-N+ membrane (Amersham), and hybridized as per standard methods with a labeled probe (Prime-It II, Stratagene) made from a 1.4 kb central fragment of the clk-2 cDNA. Identical results were obtained with two independent worm and RNA preparations.

6.1.7 Antibodies and Western Analysis

A PCR fragment encoding amino acids 401-877 was cloned into the pET16b expression vector (Novagen). Bacterially expressed His₁₀-tagged recombinant protein was purified by chromatography on Ni²⁺-NTA-Agarose, and injected into two rabbits to obtain polyclonal antibodies. The terminal bleed of each rabbit recognizes the bacterial antigen, in vitro translated clk-2, and a predominant band at the expected size of ˜100 kDa in worm extracts (prepared by grinding in NET buffer). This ˜100 kDa band is not detected by pre-immunization sera, and disappears upon pre-absorption of the antibody with bacterially purified clk-2, but not upon pre-absorption with other purified bacterially expressed proteins, including His₁₀-tagged. The same band is detected by blot-affinity purified antibodies. Also, the detected band is drastically reduced in clk-2(qm37) extracts as compared to wild type. An additional band at the expected size of ˜130 kDa is detected by these antisera and by an anti-GFP antibodies in worm extracts expressing PₕₛₚCLK-2::GFP. Western blotting was performed as per standard methods. The primary antibodies were rabbit anti-clk-2 antisera diluted 1:1000, and the secondary antibody was HRP-conjugated goat anti-rabbit IgG (Sigma) diluted 1:2000, detected using ECL detection kit (Amersham). Samples concentration was measured by BioRad Assay and equal loading was controlled by Coomassie Blue staining of an identical gel made from the same samples.

6.1.8 Telomeric Restriction Fragment Length Analysis

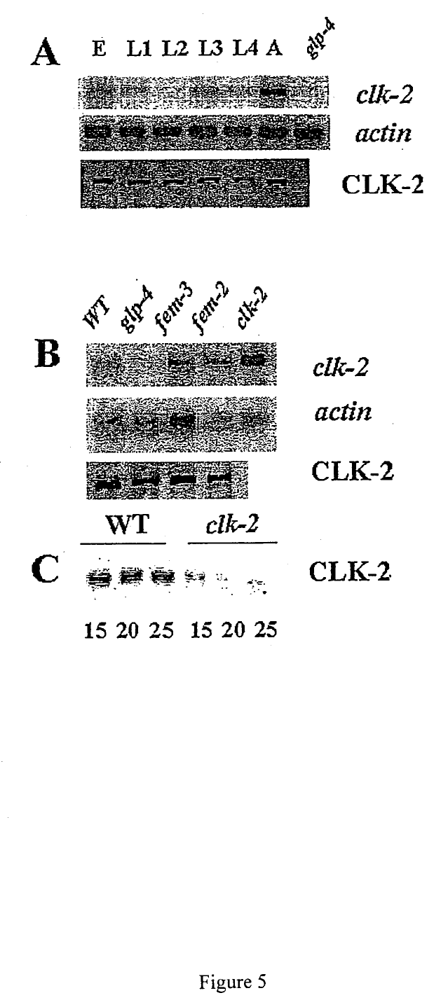

Worms were continuously grown for numerous generations at 20 and 25° C. before collection for DNA extraction. Worms were collected as mixed-stage populations for all strains examined. As clk-2(qm37) is lethal at 25° C., mixed stage worms from 20° C. were transferred to and grown at 25° C. for 3-4 days; controls thus treated were also analyzed. At least four independent preparations of worms were analyzed for each condition. Genomic DNA was phenol-chloroform purified and ethanol precipitated. 5 μg of DNA were HinfI digested, separated on a 0.6% agarose gel at 1 Vcm⁻¹, and transferred to Hybond+ under alkaline conditions. Southern blots were hybridized with two probes directed against telomeric repeats, and gave identical results: (1) a [γ³²-P]dATP end-labeled oligonucleotide TTAGGCTTAGGCTTAGGCTTAGGCTTAGGCTTAGGCTTAGGCTTAGG (SEQ ID NO:33), which was used for the blots presented in FIGS. 5A-C, and (2) a probe generated by direct incorporation of [α⁻³²P]dCTP during PCR amplification of telomeric repeats from plasmid cTel55X with primers T7 and SHP1617 (GAATAATGAGAATTTTCAGGC) (SEQ ID NO:34). We also used telomere-specific probes directed against the HinfI terminal restriction fragments, but hybridizing to non-telomeric sequence just adjacent to the terminal telomeric repeats, so that we could detect a particular telomeric terminal fragment. The terminal restriction fragments of the left telomere of chromosome IV (IVL) was detected with a gel purified 200 bp PCR product from plasmid cTel4X amplified with primers SHP1792 (ATTCCTTCTGTGTACTGTTGCC) (SEQ ID NO:35) and SHP1791 (GATATTGACGACCTAGATGACG) (SEQ ID NO:36) that was [α⁻³²P]dATP randomly labeled. The terminal restriction fragments of chromosome X (XL) was detected with a gel-purified 740 bp PCR product from plasmid cTel7X amplified with primers SHP 1797 (TCTGATTTTGACGATATTTCGC) (SEQ ID NO:37) and SHP1794 (AACTGACGGACTTGTGTCCC) (SEQ ID NO:38) that were [α⁻³²P]dATP randomly labeled.

6.2 Results

6.2.1 The clk Phenotype of clk-2 Mutants

Previously clk-2 mutants have been shown to display a phenotype similar to that of clk-1 mutants, including the maternal rescue effect, their slow development and behavior, and their increased life span (Hekimi, et al., 1995, Genetics 141:1351; Lakowski & Hekimi, 1996, Science 272: 1010). The defects of clk-2 mutants are further characterized, the results of which follow. From 15° C. to 20° C. the phenotype of clk-2 mutants is similar to that of clk-1 mutants. The average developmental, reproductive and behavioral rates are dramatically slower, and the mean and maximum life span longer, than those of the wild type as summarized in Table 3. In particular, the embryonic development of clk-2(qm37) mutants lasts 17.0±1.5 hours (n=97) at 20° C., while the wild type lasts 13.2±0.7 hours (n=80). The post-embryonic development of clk-2(qm37) mutants is also slower lasting 95.7±1.3 hours at 20° C. (n=73), while the wild-type worms take only 53.6±8.7 hours (n=184).

The defecation cycles are slowed down as well, occurring every 105.7±15.2 seconds in clk-2 mutants at 20° C. (n=10) and every 54.9±0.6 seconds in the wild type (n=70). The pharyngeal pumping rate is lower, 180.9±24.8 pumps per minute occurring in the clk-2 mutants at 20° C. (n=25), and 265.3±64.4 pumps per minute in the wild type (n=25).

TABLE 3
Phenotypic characterization of clk-2(qm37) animals at 20°

| | | | Maternally |
|---|---|---|---|
| | Wild | | rescued |
| | Type (N2) | clk-2(qm37) | clk-2(qm37) |
| Embryonic | 13.2 ± 0.7 | 17.0 ± 1.5 | 13.3 ± 1.6 |
| Development (hours) | n = 80 | n = 97 | n = 40 |
| Post-embryonic | 53.6 ± 8.7 | 95.7 ± 1.3 | 53.9 ± 12.4 |
| Development (hours) | n = 184 | n = 73 | n = 98 |
| Self-brood Size | 302.4 ± 30.5 | 83.4 | 113.9 ± 30.3 |
| (eggs) | n = 20 | n = 10 | n = 24 |
| Peak Egg-laying Rate | 5.3 | 1.3 | 3.6 ± 0.9 |
| (eggs per hour) | n = 10 | n = 10 | n = 24 |
| Defecation | 54.9 ± 0.6 | 105.7 ± 15.2 | 60.3 ± 9.0 |
| (seconds) | n = 70 | n = 10 | n = 8 |
| Pharyngeal Pumping | 265.3 ± 64.4 | 180.9 ± 24.8 | 245.2 ± 24.6 |
| (pumps per minute) | n = 25 | n = 25 | n = 11 |

In addition, the self-brood size at 20° C. have also been examined and it was found that the size is reduced in clk-2 mutants where it is 83.4 (n=10), while it is 302.4±30.5 in the wild type (n=20). The peak egg-laying rate is 1.3 (n=10) in clk-2 mutants at 20° C., and 5.3 (n=10) in the wild type. Life span of the mutants have also been examined. clk-2(qm37) mutants live longer than the wild type, living on average 22.4±7.4 days (n=100) at 20° C. and having a maximum life span of 40 days, which is longer that the average life span of 19.3±5.3 days (n=100) and maximum life span of 32 days of wild-type N2 worms.

The developmental and behavioral phenotypes are fully maternally rescued, that is to say that homozygous clk-2/clk-2 mutants derived from a clk-2(qm37)/+heterozygous mother display wild-type phenotypes. In fact, the embryonic development of homozygous mutants derived from a heterozygous mother takes only 13.3±1.6 hours (n=40) and their post-embryonic development lasts only 53.9±12.4 hours (n=98) at 20° C. Also maternally rescued are both defecation, which occurs every 60.3±9.1 seconds at 20° C. (n=8) and pharyngeal pumping, which occurs at a rate of 245.2±24.6 pumps per minute at 20° C. (n=11). However, the reproductive phenotypes are only partially rescued by a wild-type copy of the gene clk-2 in the mother. The self-brood size is 113.9±30.3 at 20° C. (n=24), and the peak egg-laying rate is 3.6±0.9 (n=24). This indicates that the wild-type clk-2 gene in the mother induces an epigenetic state that lasts for only one generation. Erasure of the epigenetic state in the germline prevents the animal from having a wild-type rate of reproduction. In addition, the life span of maternally rescued homozygous mutants is dramatically shortened vs. both the mutant and the wild-type life span. Indeed, homozygous mutants derived from a heterozygous mother live only 14.9±4.1 days on average (n=106) and have a maximum life span of 27 days at 20° C. Interestingly, wild-type siblings of maternally rescued clk-2 live slightly shorter than wild-type N2 worms, 17.3±4.1 days (n=206). This observation indicates that wild-type physiological rates imposed by a maternal epigenetic setting are deleterious to animals that are partially incapable of regulating their physiological rates in response to environmental conditions.

TABLE 4
Life span of mutants and double mutant
combinations at 20° C. indicated in day

| Genotype | Mean Life Span | Maximum Life Span |
|---|---|---|
| Wild type (N2) | 19.3 ± 5.3 | 32 |
| | n = 100 | |
| clk-2(qm37) | 22.4 ± 7.4 | 40 |
| | n = 100 | |
| Maternally rescued clk-2(qm37) | 14.9 ± 4.1 | 27 |
| | n = 106 | |
| Wild type (N2) | 18.4 ± 4.6 | 31 |
| clk-2(qm37) | 22.9 ± 7.3 | 45 |
| | n = 260 | |
| daf-16(m26) | 18.1 ± 2.6 | 25 |
| | n = 260 | |
| daf-16(m26) clk-2(qm37) | 21.7 ± 5.8 | 41 |
| | n = 260 | |
| daf-2(e1370) | 29.3 ± 10.3 | 51 |
| | n = 50 | |
| daf-2(e1370) clk-2(qm37) | 54.5 ± 21.4 | 101 |
| | n = 50 | |
| eat-2(ad465) | 30.0 ± 7.0 | 42 |
| | n = 34 | |
| eat-2(ad465) clk-2(qm37) | 26.6 ± 6.3 | 45 |
| | n = 50 | |

The life span increase produced by clk-2(qm37) is further characterized by comparing it to that produced by other aging genes as summarized in Table 4. Among the other genes that affect life span in worms, the best understood are the daf genes. Mutations in the eat genes prolong life span through caloric restriction by reducing the food intake of the animals, a process that also prolongs life span in vertebrates. Mutations in daf genes prolong life span by partial activation of the dauer formation pathway. The dauer stage is a dormant, long-lived, alternative developmental stage which is induced by adverse environmental conditions. The increased life span of all dauer formation mutants that have been tested is suppressed by loss of function mutations in daf-16.

In fact, it is found that while daf-16(m26) lives 18.1±2.6 days on average with a maximum life span of 25 days, the double mutants daf-16(m26) clk-2(qm37) lives an average life span of 21.7±5.8 days with a maximum life span of 41 days. Furthermore, although double mutants with two long-lived dauer formation mutations do not live longer than mutants carrying only one of the component mutations, daf-2(e1370) clk-2(qm37) double mutants live substantially longer than daf-2, almost three times longer than the wild type. The results show that while daf-2(e1370) lives 29.3±10.3 days on average with a maximum life span of 51 days, the double mutants daf-2(e1370) clk-2(qm37) lives an average life span of 54.5±21.4 days with a maximum life span of 101 days. In contrast to these observations, the effects of clk-2 and eat-2 are not additive. In fact, the double mutants live somewhat shorter than eat-2 mutants. The results show that eat-2(ad465) lives 30.0±7.0 days on average with a maximum life span of 42 days, and that the double mutants daf-2(e1370) clk-2(qm37) live 26.6±6.3 days on average with a maximum life span of 45 days. These observations are also consistent with the finding that daf2 eat-2 double mutants live longer than daf-2 or eat-2 mutants in isolation (Lakowski & Hekimi, 1996, Science 272:1010). Together, these results show that daf-2 and clk-2 prolong life span by distinct mechanisms but that clk-2 works in a way that resembles caloric restriction.

6.2.2 The Strict Maternal Effect of the clk-2(qm37) Mutation

In addition to the clk phenotype displayed by clk-2(qm37) mutants, they exhibit a temperature-sensitive embryonic lethal and sterile phenotypes at 25° C. It is known that qm37 is a temperature sensitive mutation and that the mutants lay dead embryos when they are transferred to 25° C. (Hekimi et al., 1995, Genetics 141:1351). These findings have now been extended, and the phenotype of clk-2 mutants at 25° C. has been examined after a number of temperature shift experiments at different stages of development, from permissive to restrictive temperature and vice versa.

At the permissive temperatures (15 to 20° C.), clk-2 embryos all develop normally and grow up to become long-lived adults. However, when hermaphrodites that have developed at a permissive temperature are transferred to 25° C. before egg-laying begins, they produce only progeny that dies during embryogenesis at various stages of development. When these hermaphrodites, that have been producing dead embryos at 25° C., are transferred back to 18° C., they lay only dead eggs at first, but start to lay live eggs that develop into adults after having been 5-6 hours at 18° C. When hermaphrodites that are kept at 18° C., and that lay only live eggs, are transferred to 25° C. it also takes 5-6 hours before they lay only dead eggs. Both conditions (laying live or dead progeny) are fully reversible upon temperature shift even when the animal's entire post-embryonic development was carried out at a single temperature (permissive or non-permissive). In addition, when larvae that developed at the perrnissive temperature are shifted to 25° C., some arrest development and others reach a sterile and sick adulthood. These phenotypes are fully reversible as well. Finally, all these lethality and sterility phenotypes displayed by clk-2(qm37) mutants at 25° C. can be fully maternally rescued: heterozygous animals produce only live progeny at any temperature.

The results also show that the embryonic lethality at 25° C. is a strict maternal phenotype. That is to say that despite qm37 behaving as a recessive mutation, a wild-type allele in the genome of the embryo is not sufficient for survival if the mother was clk-2/clk-2 homozygous mutant. When clk-2 hermaphrodites are mated to wild-type males at 25° C. they nonetheless produce only dead embryos. When shifted to 18° C. at various times after mating they produce live males, indicating that the mating was successful. The strictly maternal lethal action of clk-2 indicates a very early focus of action, before activation of the zygotic genome.

To establish how early clk-2 acts during the development of the worm, embryos at the 2-4 cell stage from wild-type N2 and clk-2 mutant hermaphrodites kept at either permissive (20° C.) or non-permissive (25° C.) temperature and transferred them to the other temperature (or not, as a control) were dissected. As summarized in Table 5, it was found that when development up to the 2-4 cell stage proceeded at the permissive temperature, almost all eggs hatched and carried out further embryonic and post-embryonic development at 20° C. {100% of dissected N2 eggs (n=35) hatched and 87% of dissected clk-2 eggs hatched (n=91)}or 25° C. {97% of dissected N2 eggs (n=36) hatched and 91% of dissected clk-2 eggs hatched (n=93)}. In contrast, when eggs had carried out development up to the 2-4 cell stage at 25° C. and were then transferred to 20° C., only very few clk-2 eggs hatched and succeeded in completing development at 20° C. {12% of dissected clk-2 eggs hatched (n=136)}. As a control, when N2 eggs had carried out development up to the 2-4 cell stage at 25° C. and were then transferred to 20° C., almost all hatched and succeeded in completing development at 20° C. {98%, n=45}, or at 25° C. {96%, n=45}. These results indicate that clk-2 is required for viability before the 2-4 cell stage. clk-2 is required in a narrow window between the very end of oogenesis and the initiation of embryonic development.

TABLE 5
Survival of eggs at the 2-4 cell stage,
dissected from mothers raised at 20 or 25° C.
and transferred or not to another temperature

| | % of eggs that hatch when | % of eggs that hatch when |
|---|---|---|
| Mothers | developing at 20° C. | developing at 25° C. |
| N2 at 20° C. | 100 | 97 |
| | n = 35 | n = 36 |
| clk-2 at 20° C. | 87 | 91 |
| | n = 91 | |
| N2 at 25° C. | 98 | 96 |
| | n = 45 | |
| clk-2 at 25° C. | 12* | n.d. |
| | n = 136 | |

Eggs that have reached the 2-4 cell stage at 20° C. are viable even when further development is carried out at 25° C., while eggs that reached the 2-4 cell stage at 25° C. die even when developing subsequently at 20° C.
*Only hatching is recorded in this table, but it should be noted that the 12% of clk-2 eggs transferred from 25 to 20° C. that succeed in hatching do not subsequently complete post-embryonic development, while the majority of the 91% clk-2 eggs that hatch when transferred from 20 to 25° C. reach adulthood.

Indeed, clk-2 hermaphrodites that have spent 26 hours of adulthood at 25° C., carry on average 9.9 developing eggs in the uterus (n=125), but produce on average 10.7 dead eggs (n=133) when shifted down to permissive temperature. This observation indicates that, upon transfer from the lethal temperature, only one oocyte or embryo dies on average in addition to those that have already formed an eggshell. This corresponds to the time at which fertilization, oocyte meiosis, pronuclear formation and eggshell formation occurs. Early embryonic development was observed using DIC microscopy but did not detect any obvious abnormality in the events which follow fertilization. The early embryos look invariably normal and healthy with cells and nuclei of normal size and shape. DNA was also visualized using Dapi in oocyte and early embryos and did not detect abnormal patterns of chromosome segregation or any other defects. Finally, meiosis per se is not affected as clk-2 homozygous males can sire abundant cross-progeny at 25° C. when mated to wild-type hermaphrodites.

6.2.3 clk-2 Positional Cloning, Gene Structure and Operon

The gene clk-2 was molecularly identified by positional cloning. The gene was localized on the genetic map within an interval of 0.84 cM on the left cluster of linkage group III of Caenorhabditis elegans, between the genetic markers sma-4 and mab-5 (Hekimi et al., 1995, Genetics 141:1351). A series of additional mapping experiments involving the genetic markers sma-3, unc-36, lin-13, and lin-39 by multi- and two-point crosses was used to refine this genetic position. The following multi-point results were obtained (the genotypes whose progeny was scored is given in brackets): dpy-17 14 clk-2 18 unc-32 (clk-2/dpy-17 unc-32); lon-1 47 clk-2 23 unc-36 (clk-2/lon-1 unc-36); sma-4 35 clk-2 3 mab-5 14 unc-36 (clk-2/sma-4 mab-5 unc-36); sma-3 18 clk-2 0 lin-13 10 unc-36 (sma-3 clk-2 unc-36/lin-13); clk-2 3 lin-13 49 unc-32 (lin-13/clk-2 unc-32); sma-3 40 lin-39 0 clk-2 33 unc-36 (sma-3 clk-2 unc-36/lin-39). In addition, a two-point cross was carried out (clk-2 unc-36/++) and 5/630 Uncs were found to develop quickly (p=0.4 cM). It was found that the deletion nDf2O does not delete clk-2 and that the duplication qDp3 does include clk-2. The gene clk-2 was thus spaced within an interval of 0.3 cM, between sma-3 (at −0.9 cM on LGIII) and lin-13 (at −0.6 cM on LGIII), and lying very close to the gene lin-39 (at −0.65 cM).

By aligning the genetic and physical maps, the physical region which likely would contain the clk-2 gene was predicted. Groups of cosmids from this region were tested for their ability to rescue the clk-2 mutant by DNA microinjection. clk-2 was rescued by a pool of 4 cosmids (H14A12, KO7D8, C34A5, C07H6). Individual injection of cosmids C07H6 and C34A5 also rescued the clk-2 phenotype, narrowing the physical position of clk-2 to within approximately 15 kb. Fragments of cosmid C07H6 (obtained by restriction digests from base pair 31,528 to base pair 36,545 of cosmid C07H6 [Accession: AC006605]) were then tested for rescue and a short region of approximately Skb was shown to fully rescue the phenotype, indicating that this 5 kb fragment contains the clk-2 gene.

The identity of the gene was further confirmed by phenocopying the clk-2 phenotype with RNA interference (RNAi) experiments, that is the injection of double stranded RNA corresponding to the coding mRNA sequence of a gene of interest to fully abolish the function of this gene. Double stranded RNA was produced by in vitro transcription from a cDNA (EST 447b4) that mapped to this region, and injected into wild-type as well as into clk-2(qm37) worms. All wild-type and clk-2 animals injected with clk-2 dsRNA initially produced embryos that hatched and developed into worms phenotypically resembling clk-2(qm37), that is, slow development, slow defecation and sterility. After 24 hours, the injected animals started laying only dead eggs. These results confirmed the identity of clk-2. The observation that RNAi-treated mothers produce dead eggs, a phenotype more severe than the weak embryonic lethality normally present in the clk-2(qm37) strain, indicated that qm37 is a partial loss-of-function mutation that displays the null phenotype only at 25° C. We further confirmed the identity of the gene by characterizing the molecular lesion underlying the clk-2 mutation. Genomic DNA from the clk-2(qm37) strain was isolated and the nucleotide sequence of the clk-2 region determined. The qm37 mutation is a G→A transition at in base 2321 of the cDNA.

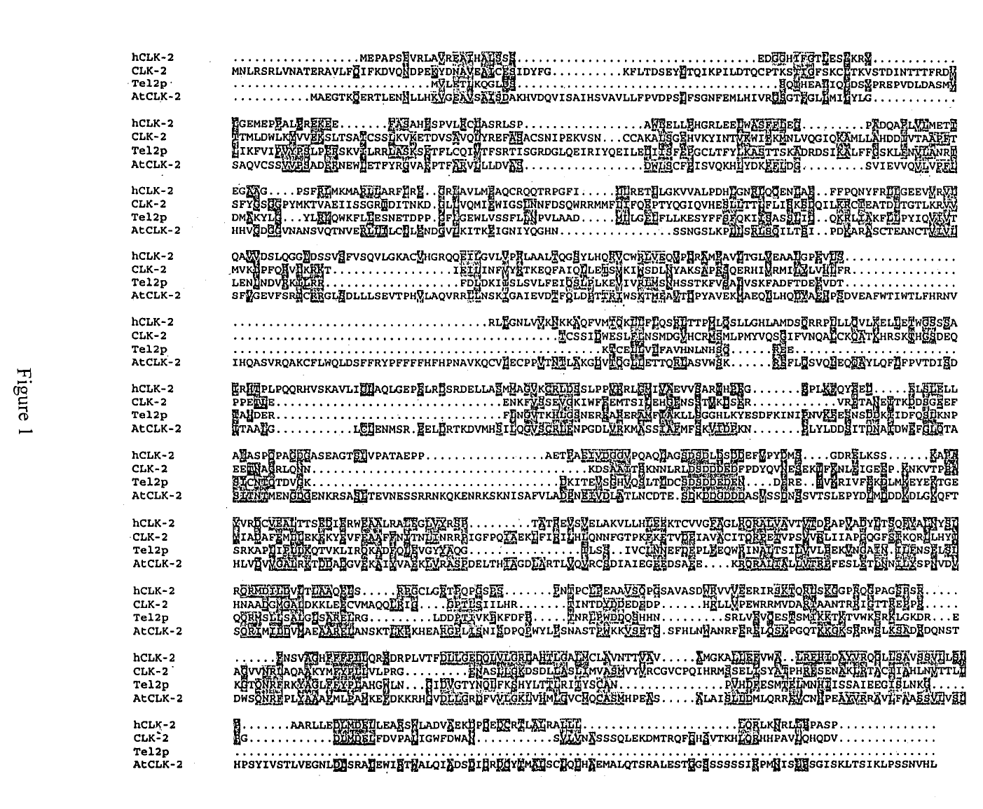

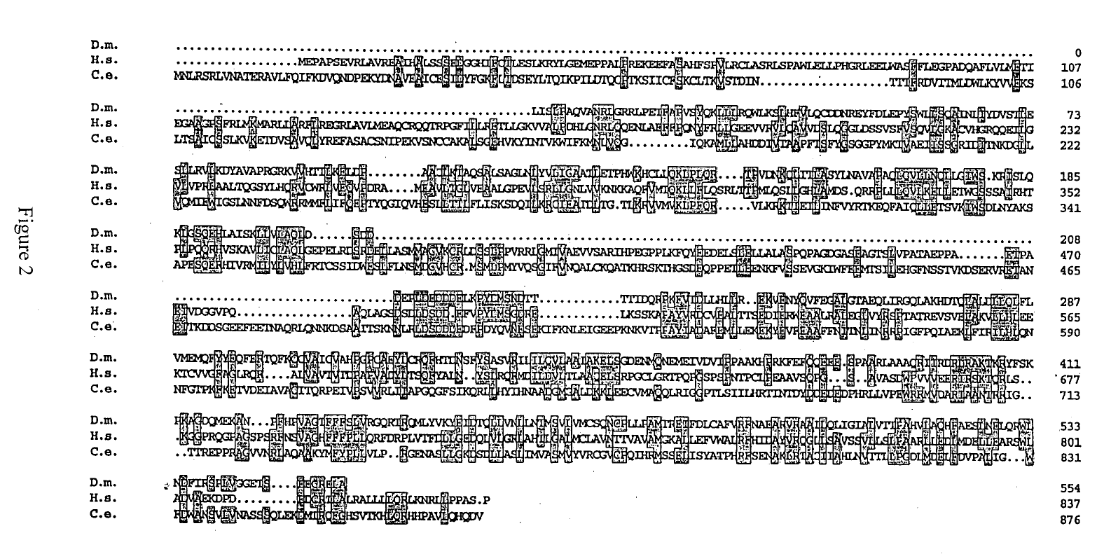

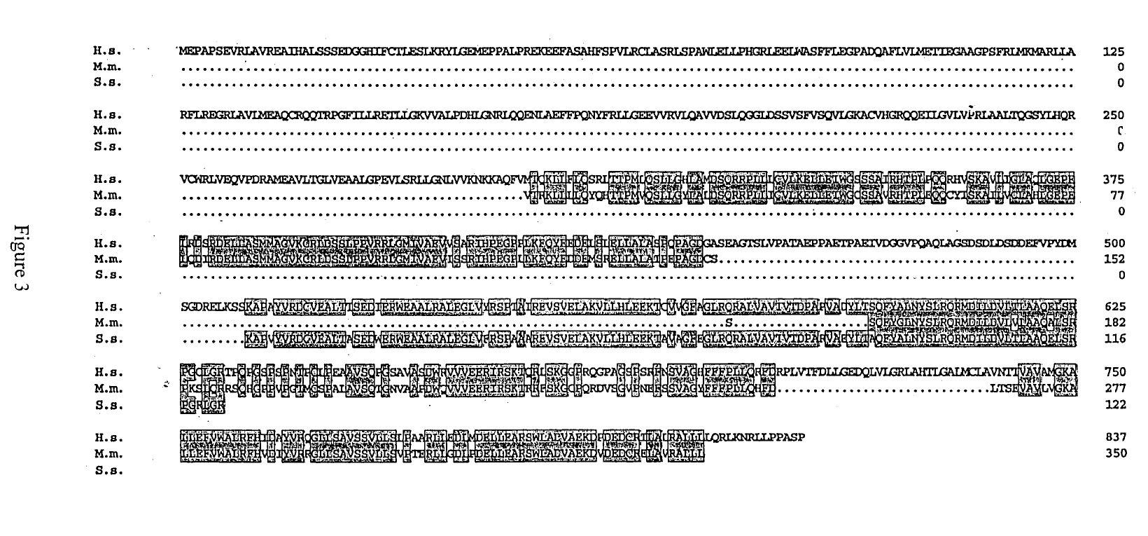

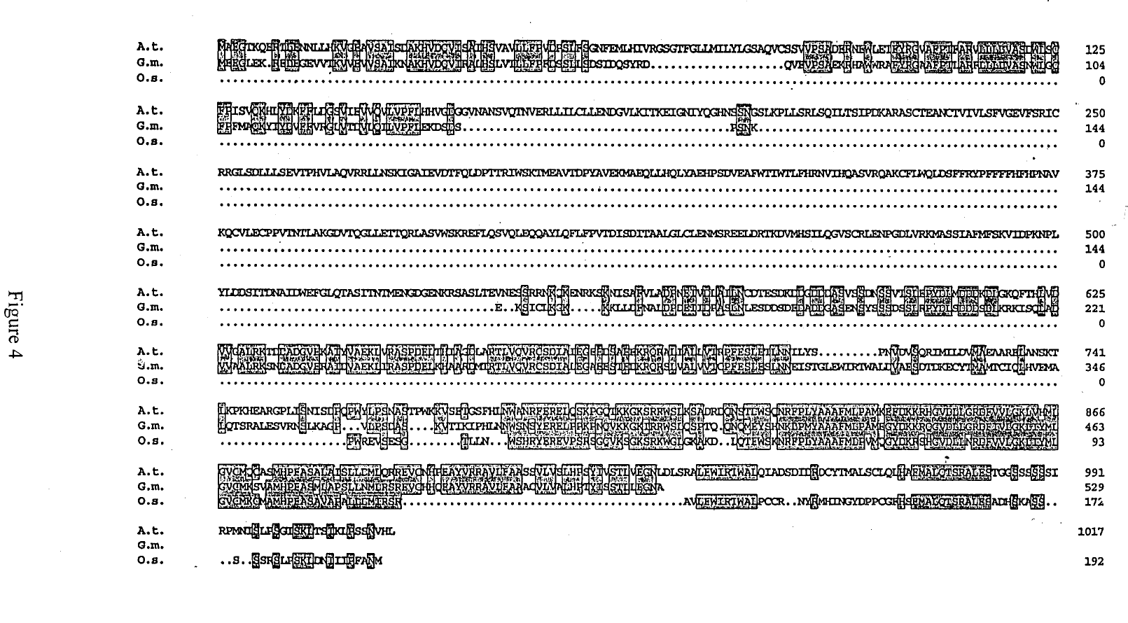

The structure of the gene was established experimentally by determining the nucleotide sequence of the EST yk447b4 cDNA, thus defining the actual intron/exon boundaries in vivo and allowing to predict the encoded protein. The gene clk-2 is SL2 transpliced. We have further established the gene structure by RT-PCR experiments, which not only showed that clk-2 is SL2 transpliced, but also that the gene just upstream to clk-2, which we called cex-7, is expressed and is SL1 transpliced. The transplicing by SL1 of a gene placed upstream, and by SL2 of a gene downstream constitutes a hallmark of genes which are in an operon, and are transcriptionally co-expressed. Therefore, clk-2 and cex-7 are transcriptionally co-expressed, and thus play functionally related roles. The cDNA (yk215f6) that corresponds to cex-7 was also sequenced. The gene cex-7 encodes a predicted protein of 481 amino acid residues in length (SEQ ID NO:39), that is similar to a human polypeptide of 550 amino acids (SEQ ID NO:40). clk-2 encodes a predicted protein of 877 amino acids and the clk-2(qm37) mutation is a cysteine to tyrosine substitution at residue 772 of the predicted protein. The expressed protein extracted from both mutant and wild-type worms at different temperatures was detected by western blot analysis. clk-2 is similar to unique predicted proteins in human (SEQ ID NO:3), Drosophila (SEQ ID NO: 13), rice (SEQ ID NO: 19), soybean (SEQ ID NO:26-30) and to Saccharomyces cerevisiae Tel2p (SEQ ID NO:32) and in other species (SEQ ID NO:7-12, 14, 17-19). The structural conservation among these proteins is illustrated by the alignment presented in FIGS. 1, 2, 3 and 4. No homologue of Tel2p had previously been recognized because aligning multiple sequences is necessary to reveal the homology. Tel2p has been shown to bind yeast telomeric DNA in a sequence-specific manner (Kota & Runge, 1999, Chromosoma 108:278; Kota & Runge, 1998, Nucleic Acids Research 26:1528) and to affect the length of telomeres.

6.2.4 Expression Pattern of clk-2

The spatial and temporal expression pattern of the gene clk-2 was determined by analyzing transcript and protein levels (FIG. 5) and by examining transgenic worms carrying reporter fusions. Panel A of FIG. 5 illustrates northern and western analyses of clk-2 at all developmental stages. The level of clk-2 mRNA appears uniform throughout pre-adult development (E, embryos; L1-L4, larval stages; A, adult; glp-4, adult glp-4(bn2ts) mutants at 25° C.). The low level of clk-2 expression in L4 larvae and in glp-4 mutants that lack a germline at 25° C. suggest that most clk-2 RNA in adults is located in gametes. In contrast to the finding with mRNA, the level of clk-2 protein is similar at all stages including adults (lower panel of A). Panel B of FIG. 5, clk-2 mRNA and protein levels (lower panel) in mutant backgrounds (glp-4(bn2ts), fem-3(q2ots), which produces only sperm at 25° C., and fem-2(b245ts), which produces only oocytes at 25° C.). The mRNA and protein levels of clk-2 expression are similar to the wild type in fem-3 and elevated in fem-2 mutants. glp-4 mutants have wild type protein levels but reduced mRNA levels. clk-2 mRNA appears strongly elevated in clk-2 mutants. Panel C of FIG. 5, clk-2 protein levels in wild type and clk-2 mutants at three temperatures. clk-2(qm37) is a missense (C772Y) and temperature-sensitive mutation. The level of clk-2 is greatly reduced in the mutant, but does not change as a function of temperature in either the wild type or the mutant. Worms were raised at 20° C. except when specified otherwise.

Populations of worms synchronized at different developmental stages were grown and total or polyA+ selected RNA was extracted from them. The highest level of clk-2 mRNA is detected in young adults. Several mutants were used to determine the origin of the transcript level in young adults. Since clk-2 mRNA level is highly reduced in glp-4(bn2ts) mutants that do not develop a germline at the non-permissive temperature, most of the RNA present in wild-type young adults is in the germline. Given the low abundance of RNA in L4 larvae which possess an already large germline but only a few male gametes, most of the clk-2 mRNA in wild-type adults is localized to meiotic gametes, in particular to oocytes.

To analyze the clk-2 protein level in different genetic backgrounds and in worms grown at different temperatures, clk-2 protein was immunodetected on western blots by using two different polyclonal antibodies, MG19 and MG20. These antibodies were obtained by injecting rabbits with a bacterially expressed polyhistidine fusion protein-His₁₀-protein. It was found that the content of clk-2 protein is uniform across developmental stages in wild type and in clk-2 animals. Furthermore, the concentration of clk-2 is not different from the wild type in glp-4 mutants which have no germline, nor in fem-3 and fem-2 mutants that contain only sperm and only oocytes, respectively. Taken together these results indicate that gametes specifically accumulate high levels of clk-2 mRNA, presumably as a store to be used by the embryo. Finally, we observed that in qm37 mutants, while the level of clk-2 mRNA appears slightly elevated, the level of clk-2 protein is greatly reduced.

Three reporter constructs of the clk-2 gene were constructed that comprised different upstream promoter regions and/or the coding region of the clk-2 gene fused to the green fluorescent protein. Two of the constructs are transcriptional fusions, one containing bases 36932 to 37319 and the other containing bases 36932 to 40010 of cosmid C07H6 [Accession: AC006605]. A third reporter construct (pMQ251) is a translational fusion that contains bases 30501 to 37319, except bases 35078 to 36545 which are part of the gene cex-7. These reporter genes were microinjected into wild type and clk-2(qm37) mutant worms, and analyzed numerous worms from several transgenic lines carrying these reporters. It was observed that the clk-2 promoter region directs expression in all somatic tissues, including hypodermis, muscles, neurons, excretory system, gut, pharynx, somatic gonad, vulva, and presumably all cells. No expression was visible in the germline, despite the use of both standard and complex array mixes. This is commonly the case for transgenes in C. elegans and does not indicate an absence of expression in the germline tissue. A full length fusion protein between clk-2 and GFP (encoded by the construct pMQ251) that complements the mutant phenotype for development, behavior and viability at 25° C., is localized exclusively into the cytoplasm, which is consistent with the absence of an obvious nuclear localization signal in the predicted protein. The pattern observed is not a consequence of overexpression as very small transgene concentrations have been used in complex arrays (Kelly et al., 1997, Genetics 146:227-238). However, although the nucleus appears dark in the fluorescent images, it still may contains very small amounts of the fusion protein. This analysis of expression indicates that clk-2 protein is indeed produced in the nematode, as shown by western analysis on total C. elegans extracts using anti-clk-2 antibodies.

Yeast Tel2p has been found to bind telomeric repeats in vitro, and thus is expected to be nuclear in vivo. However, it was found that clk-2::GFP is excluded from the nucleus. Subtelomeric silencing and telomere length regulation can also be affected by events in the cytosol. For example, Hst2p, a cytosolic NAD+-dependent deacetylase homologous to Sir2p, can modulate nucleolar and telomeric silencing in yeast (Perrod et al., 2001, EMBO J., 20:197-209), and the nonsense-mediated mRNA decay pathway appears to affect both telomeric silencing and telomere length regulation (Lew et al. 1998, Molecular and Cellular Biology, 18:6121-6130). Other proteins that affect telomere length, like tankyrase Smith et al., 1999, J. Cell Sci. 112:3649-56), are mostly extranuclear Chi & Lodish, 2000, J. Biol. Chem. 275:38437-44), with only a very small amount of protein localized to the telomeres (Smith et al., 1998, Science 282:1484-7).

6.2.5 Telomere Length

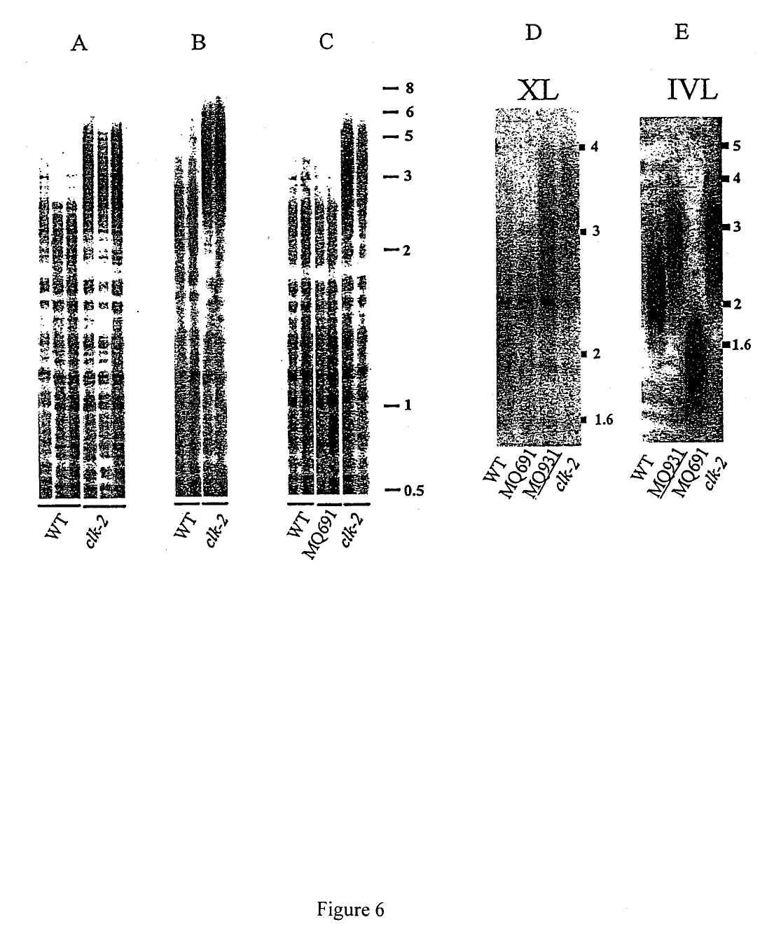

clk-2 is similar to predicted proteins in vertebrates and plants as well as to Saccharomyces cerevisiae Tel2p. Tel2p has been shown to bind yeast telomeric DNA in a sequence-specific manner, and to affect the length of telomeres. It is found that clk-2 also affected the length of telomeres in worms (FIG. 6). In worms, genomic DNA hybridization to telomeric probes after restriction digestion with HinfI reveals the end fragments of the chromosomes carrying the telomeres, which appear as smears, as well as fragments carrying tracts of telomeric repeats that are internal to the chromosome, which appear as discrete bands. Two lanes are shown for each genotype and each temperature, 18° C., 20° C., 25° C. (FIGS. 6A-C).

The length of telomeres in wild-type and clk-2 mutants was examined by Southern blotting at three temperatures, including the lethal temperature. For 18 and 20° C., worms were grown for numerous generations at each temperature before DNA extraction. Since clk-2(qm37) is lethal at 25° C., mixed stage worms from 20° C. were transferred to and grown at 25° C. for 3-4 days. Genomic DNA was prepared, HinfI digested and separated on a 0.6% agarose gel at 1.2Vcm⁻¹. Southern blots were hybridized with gamma ³²P dATP end-labeled TTAGGCTTAGGCTTAGGCTTAGGCTTAGGCTTAGGCTTAGGCTTAGG (SEQ ID NO:33) oligo-nucleotide. Use of a second type of probe, made by direct incorporation of alpha ³²P dATP during PCR amplification of telomeric repeats from the plasmid cTel55X with primers T7 and SHP1617 (GAATAATGAGAATTTTCAGGC) (SEQ ID NO:34), gave identical results. The extrachromosomal array in MQ691 clk-2(qm37); qmEx159 contains a clone with the entire coding sequence of clk-2 as well as the promoter of the operon but excluding cux-7 (bases 37319 to 31528 of cosmid C07H6, except bases 36544 to 35077) and rescues clk-2 mutant phenotypes. In clk-2 mutants, telomeres are two to three times longer than in the wild type on average. However, the chromosomes are of wild-type length in strain MQ691, which carries an extrachromsomal array expressing wild-type clk-2 in a clk-2(qm37) chromosomal background indicating that the alteration of telomere length clk-2(qm37) mutants is indeed due to abnormal function of clk-2 in these mutants.

The length of terminal telomeric fragments in the animals of the strain MQ691, which carries an extrachromosomal array (qmEx159) containing functional wild-type clk-2 that rescues development and behavior at 25° C. in a clk-2(qm37) chromosomal background, was further analyzed. A similar clone containing the qm37 mutation fails to rescue the clk-2 phenotypes. In MQ691 animals, the length of terminal telomeric fragments appear very similar to the wild-type, and even shorter, indicating that the lengthened telomere phenotype of qm37 mutants is rescued by the expression of clk-2(+). The telomere length of non-transgenic animals of the strain MQ931, derived from MQ691, which have lost the extrachromosomal array and thus again lack clk-2(+) has been further examined. The terminal telomeric repeats in this strain are long again. Thus, the lengthened telomere phenotype of clk-2(qm37) can be rescued by clk-2(+) and reverses back to mutant length after the loss of the transgene.

In C. elegans, tracks of numerous TTAGGC telomeric repeats are present at the ends of the 6 chromosomes (Wicky et al., 1996, PNAS 93:8983-8). In addition, numerous interstitial blocks of perfect and degenerate telomeric repeats are located more internally to the chromosomes (C. elegans II. Edited by Riddel et al. Published by Plainview, N.Y.: Cold Spring Harbor Laboratory Press (1997), pp 56-59, Chapter 3). Analysis of genomic DNA after restriction digestion with a frequent cutter that does not cleave within the telomeric repeats (Hinfl), electrophoresis, and hybridization to telomeric probes, reveals the telomere-carrying end fragments of the chromosomes (Wicky et al., 1996, PNAS 93:8983-8). Telomeres, and thus the restriction fragments containing them, are heterogeneous in size and appear as smears. On the other hand, restriction fragments carrying tracts of internal telomeric repeats are of fixed size and appear as discrete bands in the 0.5-3 kb range (Ahmed & Hodgkin, 2000, J. Nature, 403:159-64; Wicky et al., 1996, PNAS 93:8983-8). The quality of visualization of the length of telomeres in C. elegans with a hybridization probe that detects telomeric repeats is marred by the numerous internal repeats that also hybridize to the probe. In particular, they can mask the detection of the telomeres of chromosomes that have small HinfI terminal telomeric fragments. To further describe the telomere phenotype of clk-2(qm37) mutants, the length of individual telomeres has been characterized (FIGS. 6D-E). The subtelomeric regions just adjacent to the terminal telomeric repeats share no sequence homology among the chromosomes (Wicky et al., 1996, PNAS 93:8983-8). Taking advantage of this sequence diversity, probes specific to particular telomeres were designed. The size of a given HinfI terminal fragment is related to the fixed distance between the most exterior HinfI site of the chromosome and the beginning of the telomeric repeats, and by the variable number of terminal telomeric repeats. Upon genomic DNA digestion with HinfI and Southern blotting with a probe specific to a particular telomere, the terminal fragments, which are heterogeneous in size, again appear as a smear. Detailed results obtained for two individual telomeres, XL and IVL, are illustrated in FIGS. 6D-E.

The length of the terminal fragment of the left telomere of chromosome X is ˜1 kb longer in qm37 than in the wild type, ranging from 2.4 to 4.2 kb and from 1.7 to 2.8 kb, respectively. This telomere is of wild-type length in MQ691, which carries the rescuing transgene, and lengthens again to the clk-2(qm37) values in the non-rescued MQ931 strain. The length of another terminal fragment (left telomere of chromome IV) is also ˜1 kb longer in qm37 than in the wild type, ranging from 2.2 to 3.9 kb and from 1.8 to 2.8 kb respectively. This telomere becomes shorter than the wild type in MQ691, ranging from 1.3 to 2 kb only. This telomere acquires the mutant length again after loss of the transgene in MQ931. Thus, the overexpression of clk-2 can shorten the tracks of telomeric repeats, but not at each telomere.

6.2.6 Isolation of clk-2(qm37) Suppressors of Embryonic Lethality

clk-2(qm37) mutant C. elegans were mutagenized with EMS and then screened for those worms that suppressed the embryonic lethality normally seen at 25° C. In total, about 200,000 haploid genomes were screened in the F2 generation and about 110,000 haploid genomes were screened in the F3 generation. Five suppressors were isolated in the F2 screens, and three were isolated in the F3 screens. Overall, the frequency of suppressor isolation was ˜1 per 40000 mutagenized genomes.

The 8 candidate suppressors were kept growing at 25° C. for a number of generations. Outcrosses of the suppressed strains with dpy-17(e 164) clk-2(qm37) III indicated that all 8 suppressor mutations were dominant and linked to LGIII. Males for each of the suppressors were generated by heat shock, and mated into dpy-17(e 164) clk-2(qm37) hermaphrodites at 20° C. For each outcross, ˜12 L4 non-Dpy F1 cross progeny were singled and transferred to 25° C. The F1s were invariably fertile and produced live broods, indicating that the suppressors were dominant. Among the F2 progeny, ˜16 Dpy F2 hermaphrodites were singled at 25° C. All of them were sterile, indicating that the suppressor mutations were linked to LGIII. Also, ˜16 L4 non-Dpy F2 hermaphrodites were singled at 25° C. Some of them produced entirely wild-type broods, and corresponded to the suppressor mutation in the homozygous state.

Two of the suppressor mutations (sup) were mapped to a 0.5 cM interval of LGIII between sma-3 and unc-36 by three-factor mapping with sma-3(e491) unc-36(e251)/clk-2(qm37) sup. The genomic sequence corresponding to the clk-2 gene was sequenced for these two suppressors. A mutation in clk-2 was identified in both cases. As the other 6 suppressors were also dominant and linked to LGIII, the genomic sequence of the clk-2 gene was directly sequenced for these suppressors. All 8 suppressor mutations were intragenic and were G→A or C→T transitions typical of mutations generated by EMS. Each strain was outcrossed 3-5 times with dpy-17(e164) clk-2(qm37).

The eight independent intragenic suppressors of clk-2(qm37) fell into three classes. Six suppressor mutations were identical, resulting in an alanine to threonine amino acid substitution at residue 828 of clk-2, and belong to class A828T. Another suppressor mutation affected the same codon, but substituted the alanine for a valine (class A828V). The last suppressor mutation affected codon 859, changing a serine for an asparagine residue (class S859N).

All 3 classes of suppressors completely suppressed the embryonic lethality and sterility at 25° C., the slow developmental rates at 20° C., and the slow defecation cycles at 15° C. and 20° C., otherwise displayed by clk-2(qm37) mutants (data not shown). At 25° C. and 26.5° C., A828T suppression resulted in more efficient clk-2 function compared to A828V and S859N. In fact, A828T suppressors defecated at wild-type rates, while A828V and S859N suppressors had defecation cycles that were considerably slower than the wild type at 25° C. (each defecation cycle was on average 47.1 sec for the wild type, 49.7 sec for A828T, 64.9 sec for A828V and 60.0 sec for S859N). Also, when L1 larvae were transferred from 20° C. to 26.5° C., suppressors developed into large adults. At 26.5° C., A828T class suppressors had wild-type fertility but A828V and S859N classes of suppressors were sterile. At 27° C., none of the suppressors fully restored clk-2 function, although all suppressors were less severely affected than clk-2(qm37) mutants. When L1 larvae were transferred from 20° C. to 27° C., the suppressors developed into large sterile adults while wild-type worms were fertile and clk-2(qm37) mutants were arrested as L3 and L4 larvae.

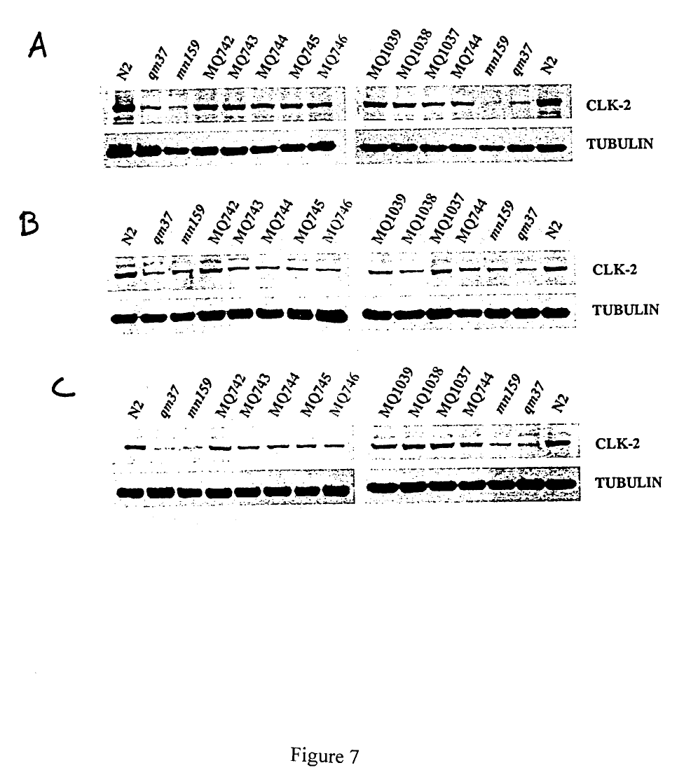

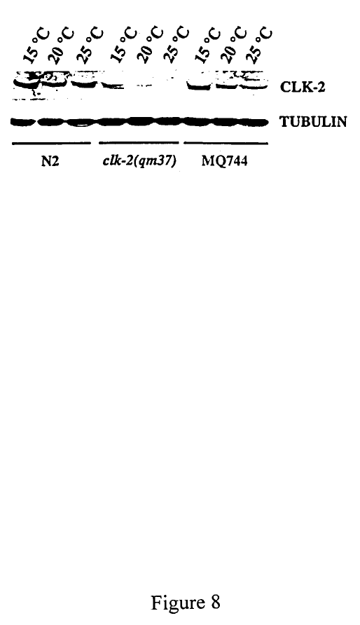

Level of the clk-2 protein was determined by immunoblot analysis of mixed-stage populations of all of the suppressors at 15° C., 20° C., and 25° C. The level of clk-2 protein was higher in the suppressors than in clk-2(qm37) mutants but lower than in the wild-type at all temperatures (FIG. 7). In particular, while clk-2(qm37) protein is undetectable at 25° C., a strong protein level was detected in the suppressors at this temperature. Thus, the clk-2(qm37-A828T), clk-2(qm37-A828V), and clk-2(qm37-S859N) proteins appear to be more stable than the clk-2(qm37) protein. The level of clk-2 protein in the A828T suppressors at 15, 20, and 25° C. was determined by immunoblot analysis. An inverse relationship was found between temperature and protein level (FIG. 8). Taken together, these observations indicate that the clk-2(qm37) suppressors cause accumulation of larger amounts of partially functional protein.

6.3 The Role of clk-2

Telomere function has been found to affect replicative life span in yeast and in vertebrate cells. It also has also been shown to affect the immortality of the germline in C. elegans. However, an involvement of telomere function in determining the life span of multicellular organisms has not been established prior to this work. Here we have shown that the maternal-effect clk-2 gene of C. elegans regulates telomere length, and prolongs life span by a mechanism that is distinct from the regulation of dauer formation but resembles caloric restriction, and encodes a protein that is similar to the yeast telomere binding protein Tel2p.

The timing of the lethal action of C. elegans clk-2(qm37) indicates a function for clk-2 during the events that immediately follow fertilization, including oocyte meiosis, pronuclei formation and karyogamy, and this would be consistent with the known importance of telomeres in meiosis. However, the examination of the morphology of chromosomes in oocytes and early embryos did not reveal any abnormalities. Similarly, although telomere function appears linked to double strand break repair and chromosome stability, including in worms, clk-2 mutants appear only moderately sensitive to ionizing radiation and do not display signs of chromosome instability. In fact, the response of clk-2(qm37) mutants to gamma-radiation was examined and found that among the progeny of irradiated animals, the proportion of dead eggs and larvae was about 10 times higher than among the progeny of irradiated wild-type animals. There is also no report of a function of Tel2p in the response to ionizing radiation in yeast.

The null phenotype of tel2 is lethal but a hypomorphic mutation of tel2 results in short telomeres and slow growth (Runge & Zakian, 1996, Molec. Cell. Biol. 16:3094). Tel2p has been shown to be involved in telomere position effect (TPE) and thus contributes to silencing of sub-telomeric regions (Runge & Zakian, 1996, Molec. Cell. Biol. 16:3094) one of the best studied examples of epigenesis. Mutations in other genes, such as tel1, that also result in telomere shortening do not result in abnormal TPE, indicating that the TPE defect in tel2 mutants is not a simple consequence of short telomeres. Furthermore, the rapid death and abnormal cellular morphology of cells fully lacking Tel2p suggests that Tel2p, like Rap1p and the Sir proteins, also functions at non-telomeric sites (Zakian, 1996, Ann. Rev. Genet. 30:141). In light of this, the absolute requirement for maternal clk-2 in embryogenesis suggests a function for clk-2 in silencing genes that are needed during some part of the worm's life cycle but that are deleterious when expressed during early development. The study of the mes genes which are required for the specification of the germline in C. elegans and can confer maternal-effect sterile phenotype has shown that mechanisms of silencing are part of the normal development of worms. Indeed, some of the mes genes have been found to encode proteins that resemble Polycomb group proteins and appear generally to be involved in the regulation of chromatin structure.

Mutations in clk-1 and clk-2(qm37) at the permissive temperature confer a similar clk-2 phenotype and in particular an increase of life span of similar magnitude (Lakowski & Hekimi, 1996, Science 272:1010) and show similar pattern of interactions with other aging genes (Lakowski & Hekimi, 1998, PNAS 95:13091). clk-1 is a mitochondrial protein of unknown function (Felkai et al., 1999, EMBO J. 18:1783). In an attempt to explain many puzzling features of the clk-1 phenotype, including the maternal effect, we have suggested that the action of clk-1 is to indirectly, but specifically, regulate nuclear gene expression (Branicky et al., 2000, Bioessays 22:48). clk-2 can be one of the molecules that implements changes in gene expression in response to alteration of clk-1 activity. clk-1 clk-2 double mutants have a phenotype that is more severe than either of the single mutants (Lakowski & Hekimi, 1996, Science 272:1010). However, the phenotype of a double mutants containing the null allele clk-1(qm30) is not more severe than a double mutant containing the much weaker allele clk-1(e2519), in contrast to the situation with clk-3, for which double mutants with clk-1(qm30) are much more severe than with clk-1 (e2519) (Lakowski & Hekimi, 1996, Science 272:1010). These observations indicate that at least part of the activity of clk-1 requires clk-2. Furthermore, clk-1 clk-2 double mutant embryos resemble clk-1 mutant in that the interphases of the embryonic cell cycles are slowed down, but mitoses appear unaltered. This indicates that clk-2 as well as clk-1 is involved in determining the rate of cellular multiplication, and thus affects mechanisms which are known to lead to cancer when deregulated.

Telomere function has also been implicated in the replicative life span of yeast, where Sir proteins mediate silencing at the telomeres and the HM loci. When displaced from the telomeres by mutation or by shortage of telomeric DNA, part of the Sir complex can move to the nucleolus where its action appears to prolong replicative life span. These and other studies indicate that telomeres are a reserve compartment for silencing factors and participate in regulating silencing in other parts of the genome. It has been suggested that the effect on cellular senescence of expressing telomerase in cultured human cells might be mediated by an effect on silencing rather than by preventing chromosome erosion. According to the Applicants, clk-2 must be involved in determining cellular senescence (in vertebrates), and also in aging and diseases linked to cellular senescence such as cancer.

### 7. EXAMPLES

The following examples demonstrate the effect of overexpression and underexpression of human clk-2 in cell lines, including the hypersensitivity to apoptosis in cells overexpressing human clk-2. The phenotypes associated with these cell lines can be used for screening for agents that can interact with and/or modulate the activity of human clk-2 protein.

7.1 Materials and Methods

7.1.1 Cell Culture

All cells were grown in high glucose DMEM supplemented with 10% fetal bovine serum (plus non essential medium amino acids for HT-1080 and SK-HEP-1) at 37° C. in an atmosphere of 5% CO₂ and 95% air. See Table 6 for a description of the origin of the cells.

7.1.2 Construction of the Plasmid pLXSH-hclk-2 and Establishment of a Stable Cell Line Overexpressing hclk-2

A cDNA clone hk02952 (insert size 4337 bp), containing the full-length hclk-2 cDNA sequence, as well as parts of intronic sequences (1929-2171, 2288-2456, 2812-3434) was obtained from Kazusa DNA Research Institute, Japan. Using this cDNA as a template, two fragments that exclude the intron sequences, Δhclk-2-A (from 256 bp to 1929 bp) and Δhclk-2-B (from 1929 bp to 3434 bp) were generated by PCR, and cloned into a pcDNA3.1/V5/His/TOPO vector (Invitrogen) to produce pcDNA3.1-Δhclk-2-A and pcDNA3.1-Δhclk-2-B. A BamHI-EcoRV fragment from pcDNA3.1-Δhclk-2-A(−) was subcloned into the BamHI-HpaI site of pLXSH (Miller and Buttimore, 1986,Mol Cell Biol 6, 2895-2902) to produce pLXSH-Δhclk-2-A. A BamHI fragment from pcDNA3.1-Δhclk-2-A(−) was inserted into the BamHI site of pLXSH-Δhclk-2-A to produce pLXSH-hclk-2.

Stable virus-producing cell lines were generated using procedures described previously (Miller et al., 1993, Methods Enzymol 217, 581-599). Briefly, the retroviral constructs were used to transfect GP+E86 ecotropic packaging cells (Markowitz et al., 1988, J. Virol. 62, 1120-1124), and viruses thus produced were used to infect the amphotropic packaging cell line PA317. Selection was performed 48 hr after infection in 400 U/ml hygromycin B and continued until colonies were visible. The colonies were pooled and expanded to establish the virus-producing cell lines.

Target cells (see Table 6) were transduced with the retrovirus as described (Lochmuller et al., 1999, Exp Cell Res 248:186-93) and selected in hygromycin at the concentrations indicated.

TABLE 6
Cell lines transduced with the retrovirus

| Name (ATCC No.) | Tissue derivation | Hygromycin U/ml |
|---|---|---|
| C2C12 (CRL-1772) | Mouse myoblast | 400 |
| Rat1-R12 (CRL-2210) | Rat fibroblast | 200 |
| A549 (CCL-18S) | Human lung carcinoma | 900 |
| SK-N-AS | Human neuroblastoma | 400 |
| SK-HEP-1 | Human liver adenocarcinoma | 400 |
| HT-1080 | Human fibrosarcoma | 400 |
| 293 | Human kidney carcinoma | 400 |
| MCH58 | Human fibroblast | 100 |

7.1.3 Construction of Plasmid pTRE₂-hclk-2 and Establishment of a Double Stable Tet-Off HT-1080 Line with Inducible hclk-2

The hclk-2 full-length cDNA sequence containing engineered NotlI and EcoR V sites was generated by PCR and inserted into the NotI-EcoR V site of pTRE₂ (Clontech, Palo Alto) to produce plasmid pTRE₂-hclk-2. The plasmid DNA was transfected into premade Tet-off HT-1080 cells (Clontech) using the superfect reagent (Qiagen).Cells were selected in 400 U/ml hygromycin 48 hr after infection and selection was continued until colonies were visible. 30 colonies were picked and immunoblot analysis using polyclonal anti-hclk-2 antibodies (see section 7.1.6 below) showed that 5 clones (nos. 3, 6, 11, 19 and 21) overexpressed hclk-2 in an inducible manner. Cells were grown in the presence of doxycyclin (1 μg/ml) to turn off hclk-2 expression, and in the absence of doxycyclin to turn on hclk-2 expression. Immunoblot analysis showed that the level of expression of hclk-2 in the Tet-off cell line (clone 21) was dependent on the dosage of doxycycline.

7.1.4 Construction of Plasmid pcDNA3.1-OCT-hclk-2 and Transient Overexpression of OCT: hclk-2 Fusion Protein in HT-1080 Cells

Mega primer PCR was used to generate a cDNA fragment encoding the OCT mitochondrial leader and hclk-2 fusion protein. The PCR product was subcloned into pcDNA3.1/V₅/TOPO to produce plasmid pcDNA3.1-OCT-hclk-2. The plasmid DNA was transfected into HT-1080 cells using the superfect reagent (Qiagen).

7.1.5 Knocking Down the Expression of hclk-2 by Sequence Specific siRNA

siRNA oligos were synthesized by Dharmacon Research (Lafayette, Colorado). siRNA duplex selection and transfection were performed as described (Elbashir et al., 2001, Nature 411, 494-498). The sequence of the siRNA (nucleotide residues 330-348 of SEQ ID NO:4) for targeting endogenous hclk-2 was as follows: sense siRNA-5′GCGGUAUCUCGGUGAGAUGdT3′, antisense siRNA-5′CAUCUCACCGAGAUACCGCdT3′. For each well of a 6-well plate, 240 pmol of siRNA duplex was used. Cells were exposed to the siRNA treatment on day 1, and they were passaged 1:4 on day 4.

7.1.6 Preparation of Polyclonal Antibodies Directed Against hclk-2 Protein

Two separate antigens were used to develop anti-hclk-2 polyclonal antibodies. The first antigen was generated as follows. A PCR fragment corresponding to bases 1516-1929 of the hclk-2 clone hk02952, encoding amino acids 414 to 551 of hclk-2, was cloned into the pGEX-3×expression vector (Pharmacia). A soluble GST-hclk-2(414-551) protein of the expected size (˜46 kDa) was expressed in DH10b bacteria and purified by affinity-chromatography on a GST slurry. This recombinant protein was injected into two rabbits (2779 and 2780) to obtain polyclonal antibodies. To generate the second antigen, a PCR fragment corresponding to bases 279-1519 of the hclk-2 clone hk02952, encoding amino acids 2 to 415 of hclk-2, was cloned into the pGEX-3×expression vector. An insoluble GST-hclk-2(2-415) protein of the expected size (˜78 kDa) was expressed in DH10b bacteria, and was purified from bacterial inclusion bodies. This recombinant protein was injected into two rabbits (2838 and 2839) to obtain polyclonal antibodies.

All four sera specifically react to hclk-2 by the following criteria. The terminal bleed of each rabbit recognizes the corresponding bacterial antigen, in vitro translated hclk-2, a band at the expected size of ˜100 kDa in cell extracts, and a strong band of the same size in cells overexpressing hclk-2 (see Table 6). This 100 kDa band is not detected by any of the pre-immune sera. Moreover, this band disappears upon preabsorption of the antibody with the corresponding purified GST-hclk-2 protein, but not upon preabsorption with other unrelated bacterially expressed proteins, including GST fusions. Also, the intensity of this band is drastically reduced in hclk-2-siRNA treated cells as compared to controls. The serum from rabbit 2780 gave the strongest reaction and was used throughout this study.

7.1.7 Immunostaining

Cells were transiently transfected with a plasmid and 24 hr later, they were seeded on coverslips. Forty-eight hours later, the coverslips were fixed in 4% paraformaldehyde/PBS for 10 min, permeabilized in acetone for 3 min, then incubated at room temperature for 1 hr with rabbit polyclonal anti-hclk-2 (1:100-1000), followed by biotinylated goat anti-rabbit or mouse IgG (1:5000) for 1 hr. Finally, the cells were incubated with fluorescein-conjugated streptavidin (10 μg/ml) for 30 min and viewed under a Leitz fluorescence microscope.

To visualize mitochondria living cells on coverslips were first incubated for 30 min in DMEM medium containing Mitotracker Red CMXRos (Molecular Probes), followed by incubation in fresh DMEM for 3×10 min. The coverslips were then fixed in 4% paraformaldehyde/PBS and stained for hclk-2. Similar results were obtained with 2780 and 2838 sera.

7.1.8 Immunoblot Analysis

Cultured cells were trypsinized and pelleted, then resuspended in 5× volumes of extraction buffer [500 mM NaCl, 20 mM Tris 8.0, 1% NP-40, 1 mM DTT and protease inhibitors (Roche Diagnostics, Mannheim)]. The resuspended cells were submitted to 5 freeze-thaw cycles (frozen in liquid nitrogen and thawed at 37° C.). Cell debris were removed by centrifugation and the quantity of protein was measured (BioRad protein assay).

50 μg of protein were separated on 7.5% or 12% polyacrylamide gels and transferred to nitrocellulose. The membranes were preincubated in blocking solution (TBST+5% non-fat milk) at room temperature for 1 hr, then incubated with the primary antibody at 4° C. overnight at the following concentrations: rabbit anti-hclk-2 antibody (1:500 to 1:1000), mouse anti-tubulin antibody (1:10000, Sigma), rabbit anti-actin antibody (1:500, Sigma), mouse anti-cytochrome C (1-2 μg/ml, Molecular Probes) and mouse anti-p300 (2 μg/ml, Upstate Biotechnology). After 3×15 min TBS-T washes, the membranes were incubated in blocking solution at room temperature for 2 hr. The membranes were then incubated with donkey anti-rabbit IgG secondary antibody (1:3000, Jackson Immunoresearch Laboratories), or goat anti-mouse IgG (1:10000, Pierce) at room temperature for 1 hr, followed by 3×15 min TBS-T washes. Finally, the signal was detected by chemoluminescence (Amersham).

7.1.9 Growth Rate/Death Assays

Cells were seeded in 6-well dishes at 1×10⁵/well. At the times indicated, they were trypsinized and counted with a haemocytometer to determine growth rate.

To determine the occurrence of cell death, cells were seeded at 1×10⁵ in 6-well dishes. The next day the cells were treated by γ-ray (20 Gy) and counted 72 hr later. A series of different apoptosis-inducing agents were also investigated and the cells were analyzed at various times following treatment (see Table 7). Cell viability was measured by the trypan blue exclusion method.

TABLE 7
Cell death assay

| | Working | | Time of |
|---|---|---|---|
| Treatment | concentration | | treatment (hr) |
| Etoposide | 100 | μM | 24 |
| Sodium azide | 15 | μM | 48 |
| Menadione | 12 | μM | 24 |
| Anisomycin | 2 | μM | 16 |
| t-Butyl hydroperoxide | 40 | μM | 48 |
| Staurosporine | 2 | μM | 24 |
| All-trans-retinoic acid | 4 | μM | 96 |
| Hydrogen peroxide | 0.5 | mM | 24 |
| Juglone | 0.5 | μM | 24 |
| Hydroxyurea | 0.6 | mM | 96 |
| Tunicamycin | 5 | μg/ml | 24 |

7.1.10 Measuring the Length of Telomeres

Genomic DNA from cultured cells was recovered by phenol-chloroform extraction and ethanol precipitation. 10 μg DNA was digested by HinfI and RsaI (10 U/μg DNA) at 37° C. overnight. The completely digested DNA was separated on 0.7% agarose gel at 23 V for 24 hr and transferred by capillary transfer to a positively-charged nylon membrane (Amersham) overnight. The telomere specific sequence (5′-TTAGGGTTAGGGTTAGGG-3′) (SEQ ID NO:41) was used as a probe to detect telomeric repeats. The membrane was incubated in pre-hybridization solution (5×SSC, 5′Denhardt's, 0.1% SDS) for 1 hr at 50° C., followed by an overnight incubation in hybridization solution (5×SSC, 0.1% SDS and 5′-³²P-end labeled probe) at 37° C. The membrane was then washed in 3×SSC, 0.1% SDS at 42° C. for 3×10 min and exposed at room temperature overnight.

7.1.11 Preparation of Subcellular Fractions

Subcellular fractionation was performed as described (Krajewski et al., 1993, Can. Res. 53:4701-14). From 1-10×10⁷ cells were washed twice with ice-cold PBS and resuspended in buffer (0.25 M sucrose, 10 mM Tris-HCl pH 7.5, 1 mM EDTA, protease inhibitors (Roche Diagnostics, Mannheim) at a concentration of 2×10⁷cells/ml. Cells were homogenized on ice (10-20 strokes at 1000 rpm, Potter-Elvehjem) until 95% of the cells were lysed based on trypan blue dye uptake. The samples were transferred to 1.5 ml Eppendorf centrifuge tubes (1 ml/tube) and centrifuged at 500 g for 5 min to pellet the nuclei. The nuclear pellet was then resuspended in 0.5-2 ml of 1.6 M sucrose containing 50 mMTris-HCl pH 7.5, 25 mM KCl, 5 mM MgCl₂. After underlayering with 1-2 ml of 2.0-2.3 M sucrose containing the same buffer and centrifugation at 150,000 g for 60 min, the resulting nuclear pellets were resuspended in 0.1-0.3 ml of 1% Triton X-100-containing buffer (0.15 M NaCl, 10 mM Tris (pH 7.4), 5 mM EDTA, 1%Triton X-100). The supernatant resulting from the initial low-speed centrifugation was subjected to centrifugation at 10,000 g for 15 min at 4° C. to obtain the heavy-membrane (HM) fraction (a pellet that should include, mitochondria, lysosomes, Golgi, and rough endoplasmic reticulum). The supernatant was centrifuged for 60 min at 15,000 g to obtain the light-membrane (LM) fraction (a pellet that should include the smooth and rough endoplasmic reticulum) and the cytosolic fraction (supernatant). The HM and LM fractions were resuspended in 1%Triton-containing lysis buffer. An equal amount of protein (50 μg) from each fraction was analyzed by immunoblot.

7.2 Results

7.2.1 Growth Stimulation by Overexpression of hclk-2 in SK-HEP-1 Cells

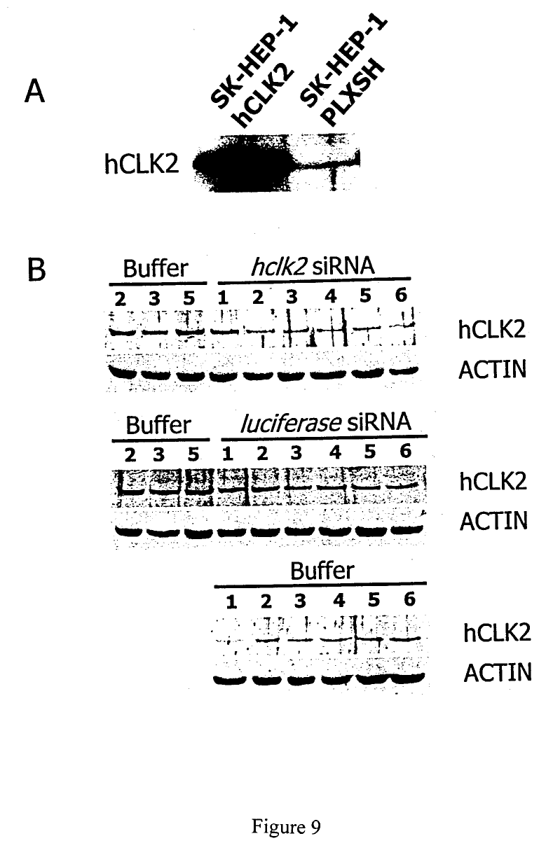

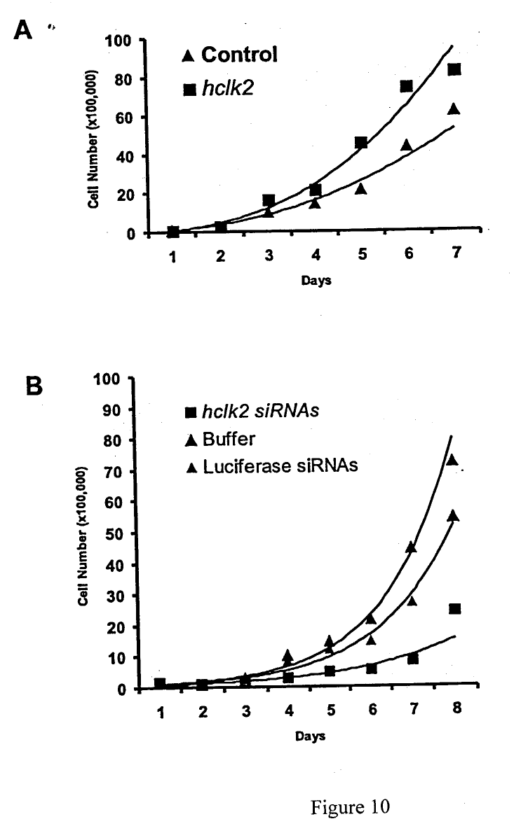

To study the broad pleiotropy of clk-2 mutations, the function of the human clk-2 homologue (hclk-2) in SK-HEP-1 human hepatoma cells was investigated. To achieve high levels of hclk-2 expression in cultured cells, a retroviral vector expressing hclk-2 was used to infect a panel of cell lines (see section 7.1.2) and stable cell lines infected with the vector expressing hclk-2 or the empty vector control were established. A high level of hclk-2 expression was detected in all the established cell lines (FIG. 9A). In every case, the cells expressing hclk-2 did not show any morphological alterations compared to controls. However, it was observed that the growth rate of SK-HEP-1 (Heffelfinger et al., 1992; In Vitro Cell Dev. Biol. 28A: 136-42) cells overexpressing hclk-2 (FIG. 10A) was increased over the control line (FIG. 10B), indicating that growth rate is sensitive to the level of hclk-2. SK-HEP-1 cells were used for all subsequent characterization of the function of hclk-2.

7.2.3 Reducing the Level of hclk-2 Expression Causes Reversible Growth Arrest

To investigate the consequences of a loss of function of hclk-2, the small interfering RNA (siRNA) technique (Elbashir et al., 2001; Nature 411:494-8) was used. SK-HEP-1 cells were treated with either 1) hclk-2-specific siRNA, 2) siRNA for luciferase, a gene that is not normally found in human cells, or 3) the same volume of siRNA annealing buffer. The level of hclk-2 and the cell number were determined daily for 7 days following siRNA treatment (FIGS. 9B and 10B). The immunoblots demonstrate that when the cells were treated with hclk-2-specific siRNA the level of hclk-2 was significantly decreased by day 2 and remained low until at least day 6. As expected, neither luciferase siRNA nor siRNA annealing buffer alone, resulted in a decrease of the expression of hclk-2. In addition, the expression of actin was not affected by hclk-2-specific siRNA, luciferase siRNA, or siRNA annealing buffer alone (FIG. 10B).

hclk-2 siRNA treatment dramatically slowed cellular growth rate, in contrast to treatment with luciferase siRNA, which had only a minor effect (FIG. 10B). The effect on growth rate lasted until day 7, after which time the cells appeared to recover from the treatment and resumed growth. No increase in cell death or other obvious changes were observed, indicating that the arrest was not the consequence of major damage to the cells.

Treated cells were also sorted by FACS according to DNA content. The arrested cells treated with hclk-2 siRNA did not appear to be arrested in any particular phase of the cell cycle.

It was found that knocking down hclk-2 levels with siRNA treatment almost completely arrests the cell cycle, and that overexpressing hclk-2 shortens cell doubling time. This finding indicates that the activity of hclk-2 is necessary for cell cycle progression and that the level of hclk-2 is limiting for cell cycle progression, at least in SK-HEP-1 cells. As the cells do not appear to arrest in any particular phase of the cycle, hclk-2 is likely not associated with any of the particular mechanisms that allow cells to pass from one phase to the next, such as DNA damage checkpoints. In worms, the partial loss of function of clk-2 leads to a failure to arrest the cell cycle in response to radiation and hydroxyurea injury. This cannot be compared directly to the results just described with hclk-2 because it is not known in what aspects of the function of clk-2 that have been lost, and that have been retained, in the two temperature-sensitive point mutants that have been characterized in C. elegans.

7.2.4 Overexpression of hclk-2 Produces Hypersensitivity to Apoptosis Triggered by Oxidative Stress or DNA Replication Block

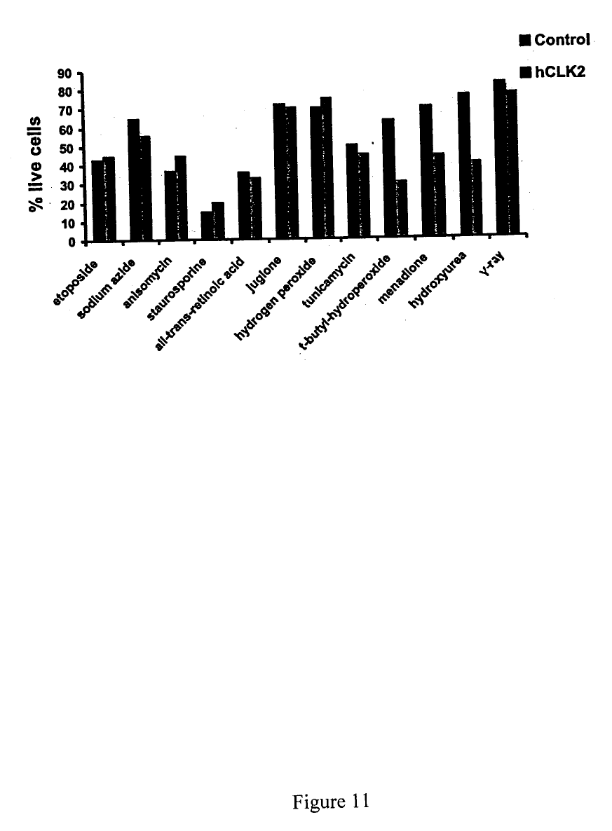

Prompted by the finding in the germline of C. elegans, where clk-2 mutations affect the response to ionizing radiations and to a DNA replication block induced by hydroxyurea (HU), the response of SK-HEP-1 cells overexpressing hclk-2 to 10 different agents capable of inducing apoptotic cell death, as well as to HU and γ-rays were investigated. The cells overexpressing hclk-2 did not show any general increase in sensitivity to apoptotic stimuli but were specifically hypersensitive to two methods of increasing oxidative stress: menadione treatment, which leads to intracellular overproduction of superoxide (Jamieson et al., 1994, Microbiology 140:3277-3283), and t-butyl hydroperoxide treatment, which leads to the production of the highly toxic hydroxyl radical (Sano et al., 1994, J. Toxicol. Environ. Health 43:339-350) (FIG. 11). The cells were also hypersensitive to the DNA synthesis inhibitor HU (FIG. 11). To verify that the cell death observed was indeed apoptotic, we stained the cells using the TUNEL method (Desjardins and MacManus, 1995, Exp. Cell. Res. 216:380-387), which consists of in situ labeling of the 3′-OH ends of the cleaved DNA typical of apoptotic cells. A significant increase in TUNEL-positive staining was observed in the nuclei of the cells treated with the compounds that produced increased cell death compared to controls After ionizing irradiation (IR) treatment, a sharp increase in apoptosis is observed in the meiotic phase of the germline of wild-type worms (Gartner et al., 2000, Mol. Cell 5:435-43; Ahmed et al., 2001, Curr. Biol. 11:1934-44). This response, however, is mostly abolished in clk-2 mutants. No corresponding increased sensitivity to irradiation in SK-HEP-1 cells overexpressing hclk-2 was found. However, a substantial increase in sensitivity to compounds that induce apoptosis by increasing oxidative stress was observed. This is interesting as the major mechanisms by which irradiation damages biological macromolecules is through the generation of reactive oxygen species.

Hydroxyurea (HU) prevents normal DNA replication (Yarbro, 1992, Semin Oncol 19:1-10) and treatment with this compound arrests the mitotic cell cycle in the germline of wild-type worms but not in clk-2 mutants (Ahmed et al., 2001, Curr. Biol. 11:1934-44). hclk-2 overexpressing cells are hypersensitive to HU and undergo apoptotic death in response to treatment with this compound. It should be noted, however, that HU is also an oxidating agent and that its effect in this system might be similar to that of other compounds that generate reactive oxygen species.

As there is no evidence to suggest that an increased sensitivity of the overexpressing cells to agents or treatments can damage DNA directly, such as etoposide (an inhibitor of topoisomerase) and IR, it is possible that the failure to respond appropriately to IR and HU in worms does not reveal a specific defect in a DNA-damage checkpoint but is the result of a decreased sensitivity to oxidative stress and/or a failure to respond appropriately to oxidative injury.

7.2.5 Overexpression of hclk-2 Gradually Lengthens Telomeres

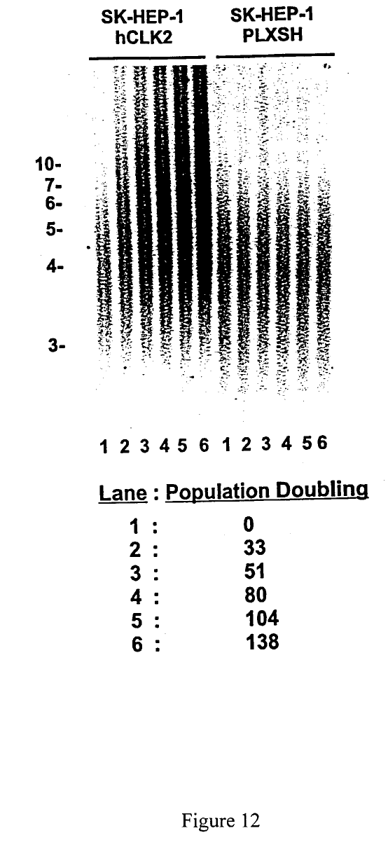

To investigate whether hclk-2 affects telomere length in human cells, as it does in S. cerevisiae and in C. elegans, the telomere length of SK-HEP-1 cells overexpressing hclk-2 and of SK-HEP-1 control cells was determined by Southern blot analysis. The telomere length at regular intervals during prolonged culturing was examined (138 population doublings, FIG. 12). The telomere length of the cells overexpressing hclk-2 gradually grew longer at an average rate of 15 bp/population doubling, while it remained absolutely stable in the control cells (FIG. 12). Ongoing culturing of the cells will indicate in the future whether the telomeres eventually stabilize at some maximum length.

In worms, the partial loss-of-function clk-2(qm37) mutation produces an overall lengthening of telomeres. However, in yeast, the tel2-1 mutation produces a gradual decrease in telomere length. In the SK-HEP-1 hepatoma cells, overexpression of hclk-2 clearly increases telomere length suggesting that a loss-of-function of the gene hclk-2 would shorten telomeres, as it is the case in the yeast tel2-1 mutant, but contrary to the clk-2(qm37) mutants in the worm. However, when telomere length is examined in worms, the DNA is extracted from whole worms at a variety of developmental stages. Overall, a lengthening of telomeres is observed, but, if the telomeres of a minor cell type were affected differently, this would probably not be detected. On the other hand, the SK-HEP-1 hepatoma cells represent a single cell type. One view, therefore, is that the Tel2p/CLK-2/hclk-2 protein is involved in a network of processes that ultimately impinge on telomere length, and that this network's reaction to perturbation might depend on the organism or cell type. Note that the telomere lengthening is very gradual, which suggests that telomerase is the ultimate effector of length changes, and not other mechanisms such as alternative lengthening of telomeres (ALT) (Henson et al., 2002).

7.2.6 hclk-2 is Present in Most Compartments of the Cell

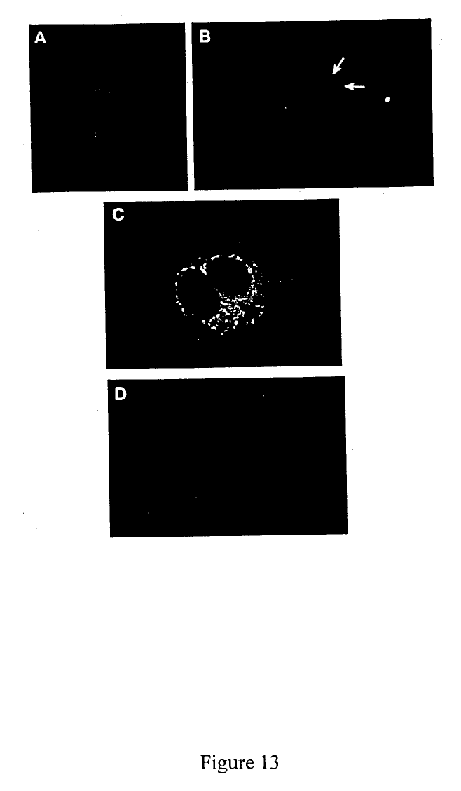

To determine the subcellular localization of hclk-2, immunocytochemistry was used to detect native and overexpressed hclk-2 in SK-HEP-1 cells. The level of native hclk-2 appeared to be too low to be detectable by this method with our antisera. However, in cells overexpressing hclk-2, the signal appeared to be everywhere in the cell, filling both the cytoplasm and the nucleus (FIG. 13A). The same distribution was also observed in another overexpressing cell line HT-1080 (FIG. 13B).

To clarify whether this ubiquitous distribution of hclk-2 was a non-specific result of overexpression, we expressed hclk-2 in the HT-1080 cell line (Rasheed et al., 1974, Cancer 33:1027-33) under an inducible promoter. Expression in these cells produced the same ubiquitous expression at all levels of induction, over a >20-fold range. Further, the distribution of a mitochondrially targeted hclk-2 fusion protein was examined. When hclk-2 fused to the ornithine transcarbamylase (OCT) mitochondrial targeting sequence (Argan & Shore, 1985, Biochem Biophys Res Commun 131:289-98) was expressed, the OCT-hclk-2 fusion was observed to be localized to the mitochondria (FIGS. 13C, 13D), indicating that the ubiquitous distribution of hclk-2 is not inherent to transgenic overexpression.

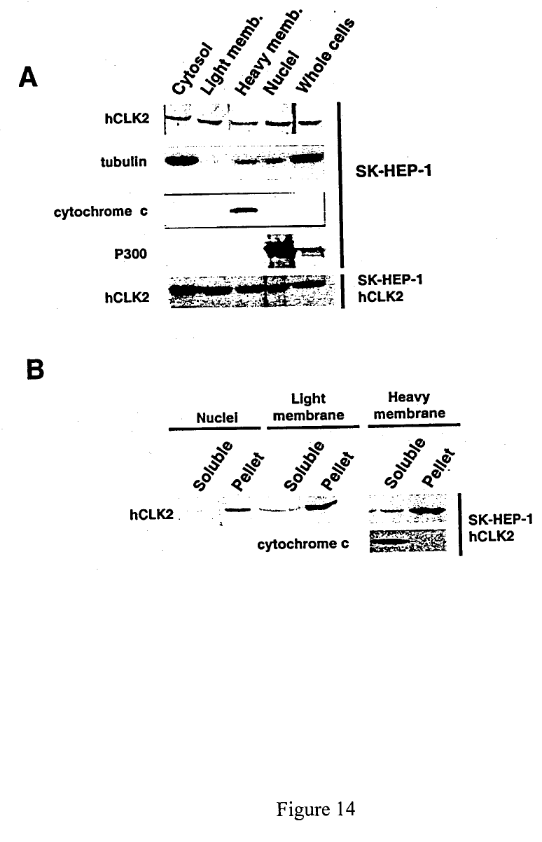

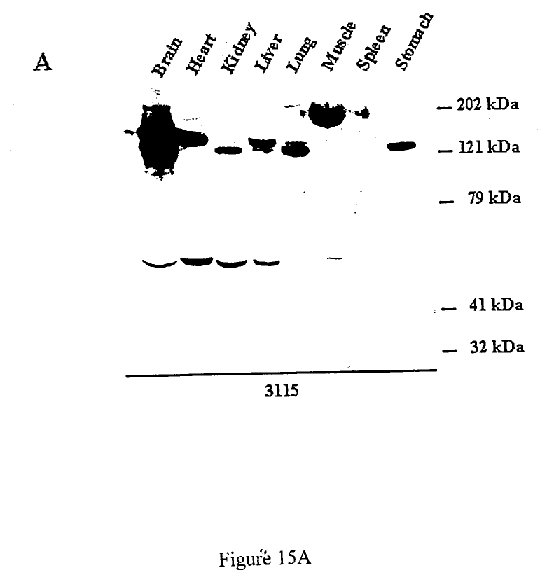

As an independent test of subcellular distribution of hclk-2, subcellular fractionation and immunoblot analysis was carried out. It was found that both native and overexpressed hclk-2 in SK-HEP-1 cells were present in all subcellular fractions, including in the nuclear, heavy-membrane (which includes mitochondria), light-membrane and cytosolic fractions (FIG. 14A). Surprisingly, the level of hclk-2 in each fraction was very similar for both native and overexpressed hclk-2. As an identical amount of protein is loaded on the gel for each fraction, this demonstrates that the concentration of hclk-2 relative to other proteins is very similar in all compartments.

Furthermore, hclk-2 is present both as a soluble and a membrane-associated form. Many proteins can have multiple cellular locations. For example, Bcl-2 is localized in the outer mitochondrial membrane, the nuclear envelope, and in the endoplasmic reticulum membrane. Also, yeast major adenylate kinase (Adk1p/Aky2p) is both mitochondrial and cytosolic. Moreover, some proteins can shuttle between different locations depending on signaling events. For example, catenin and associated proteins can be cytoskeletal, cytoplasmic, or nuclear. However, there appears to be no previous example of a protein present in such many different cellular compartments at the same time and in similar amounts.

At least one form of the hclk-2 protein is clearly soluble since it is present in the cytosolic fraction. To characterize hclk-2 in the membrane fractions, alkaline sodium carbonate was used to treat the nuclear, heavy membrane and the light-membrane fractions from overexpressing SK-HEP-1 cells. Interestingly, most of hclk-2 cannot be extracted by sodium carbonate and is detected in the pellets (FIG. 14B), indicating that hclk-2 is relatively tightly associated with the membrane in all three types of subcellular fractions. As is also evident from FIG. 14B, there is no substantial difference in molecular size between soluble and membrane-associated hclk-2. Note that although the immunocytochemical analysis indicated that hclk-2 is found in the nucleoplasm (FIG. 13), this analysis is not quantitative. From fractionation studies conducted thus far, it can be concluded that most nuclear hclk-2 is associated with the nuclear membrane, consistent with the pattern that was observed in the immunofluorescence studies (FIG. 13B).

7.3 Discussion

The study of mutants of tel2, the S. cerevisiae homologue of hclk-2, have implicated the gene product Tel2p in the regulation of telomere length and sub-telomeric silencing, as well as in an undetermined function necessary for cell viability. In worms, clk-2 mutations have been shown to affect numerous processes, including organismal features such as organized embryonic development, developmental rate, behavioral rates and reproduction, as well as cellular features such as the apoptotic death and mitotic arrest responses to irradiation and DNA replication block as reviewed in Benard, 2002, Mech. Ageing Dev 123: 869-880.

Overexpression of the hclk-2 protein decreases the population doubling time (FIG. 10A), and that knocking down the expression of hclk-2 with small interfering RNAs (siRNA) produces reversible growth arrest (FIG. 10B). Overexpression of hclk-2 also results in an increased apoptotic response to oxidative stress and hydroxyurea (HU) treatment but not to other treatments that induce apoptosis (FIG. 11). Finally, overexpression of hclk-2 gradually, but dramatically, increases telomere length (FIG. 12). These findings indicate that clk-2 and its homologues affect the same set of cellular processes in yeast, worms and humans, and suggest the possibility that it also affects in humans a comparable set of organismal processes that are affected in worms, such as life span, cell cycle control, apoptosis and telomere length regulations.

The inventor believes that hclk-2 participates in a form of membrane homeostasis. The exact composition of each membrane leaflet determines structural properties of membranes, as well as the function of membrane proteins. Both soluble and membrane-associated hclk-2 could bind a membrane lipid, aid its integration into, and regulate its abundance in the membrane. Membrane lipids are small and relatively abundant compared to proteins, which would help to explain the relatively large pool of soluble hclk-2, which would bind the non-membrane pool of the lipid.

### 8. EXAMPLES

The following examples demonstrate the expression pattern and phosphorylation of the mouse clk-2.

8.1 Materials and Methods

8.1.1 Preparation of Polyclonal Antibodies Directed Against mclk-2 Protein

An antigen was prepared for the development of polyclonal antibodies directed against mouse clk-2. Antigen pCB74 was a fusion between GST and amino acids 418-692 of the mclk-2 protein with a predicted molecular weight of ˜58 kDa. Expression of a ˜58 kD protein was obtained after induction with 1 mM IPTG for 3 hours at 37° C. after purification from bacterial inclusion bodies. Two rabbits were injected with the purified protein. The bleeds of both rabbits were tested against 50 μg of cell extracts or mouse organ extracts.

8.1.2 Tissue Extractions

Tissue was homogenized in 5 volumes of extraction buffer (500 mM NaCl, 20 mM Tris-Base pH 8.0, 1% NP-40, 1 mM DTT and protease inhibitors) using the PowerGen homogenizer (10-20 up-down strokes at power 2; Fisher Scientific). To extract the proteins, the homogenates were then subjected to 5 successive cycles of freezing (in liquid nitrogen) and thawing (in a 37° C. water bath).

8.2 Results

8.2.1 Immunodetection of mclk-2 in Mouse Tissues

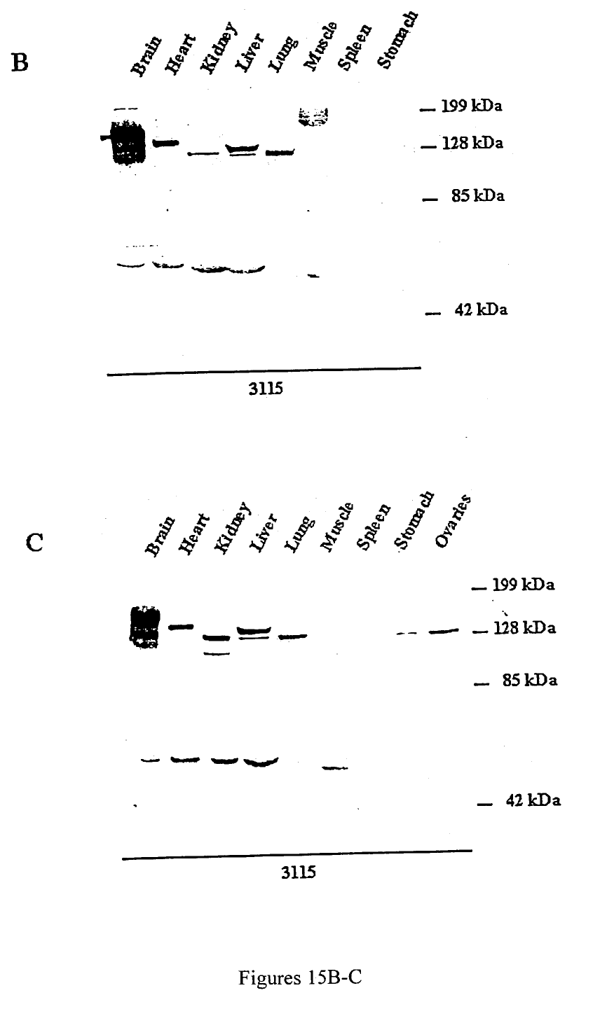

mclk-2 expression in mouse tissues was assessed by extracting proteins from different tissues of a number of adult mice. The anti-mclk-2 antibody 3115 recognizes two bands in almost all the examined mouse tissues: one main band of about 130 kDa, which is the expected size for mclk-2 and one band of approximately 60 kDa. The former band migrates slightly higher in the heart and the liver than in the other tissues. In the muscle, a strong band of ˜200 kDa is also detected (FIGS. 15A-C).

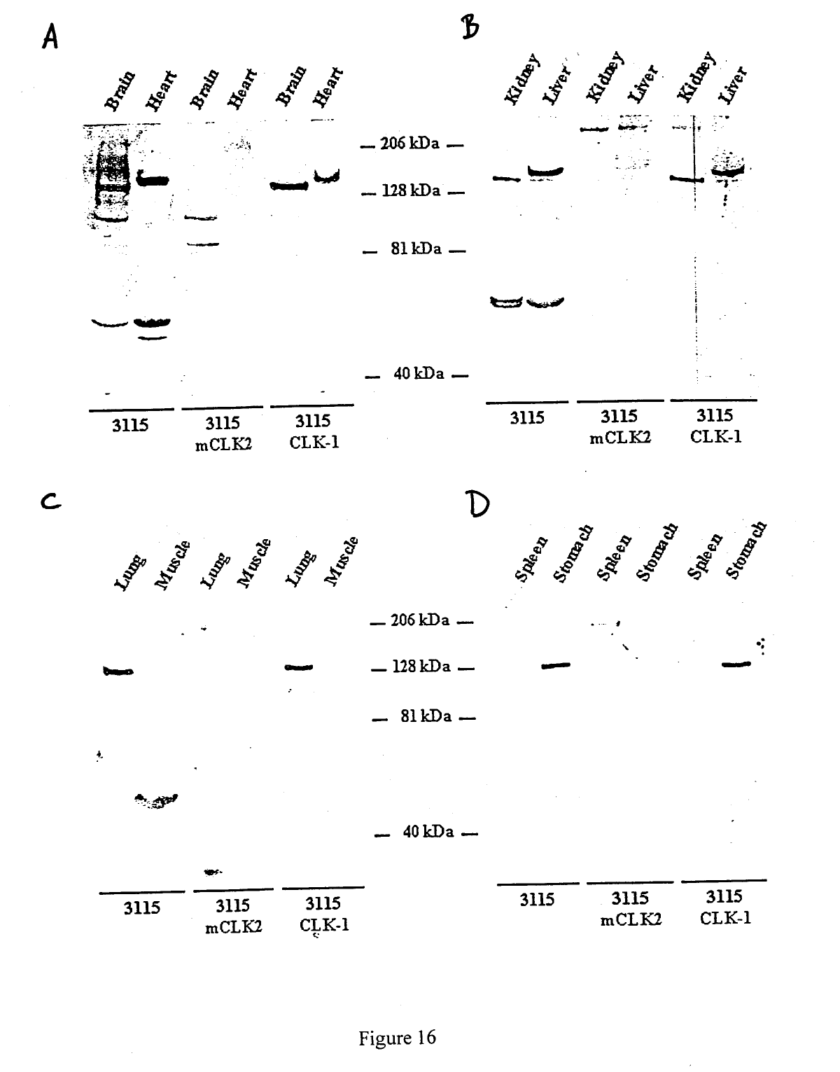

Competition experiments were carried out in order to confirm that the 130 kDa band corresponds to mclk-2. The anti-mclk-2 polyclonal antibody 3115 was pre-absorbed with the GST-clk-2 fusion protein (pCB74) used as antigen to generate the polyclonal antibody or with an unrelated GST-fusion protein prior to immunoblotting protein extracts of various mouse tissues. The results of the competition experiment are given in FIGS. 16A-D. The 130 kDa band disappears only upon preabsorption with the GST-clk-2 fusion protein, indicating that this band corresponds to mclk-2. The ˜60 kDa band disappears after pre-absorption of the antibody with either GST fusion protein, and therefore it is not specific to mclk-2. Thus, the mouse clk-2 protein is broadly expressed in a variety of tissues of the mouse.

The bleeds of rabbits 3115 and 3130 were tested against mouse fibroblast cells GPE-86 and against GPE-86 cells overexpressing human clk-2. The anti-mclk-2 antibodies recognize a strong band of an expected size (˜100 kDa) in GPE-86 cells overexpressing the hclk-2. A faint band, corresponding to the endogenous mclk-2, is also detected by antibody 3115 in GPE-86 cells. Thus, anti-mclk-2 polyclonal antibodies 3115 and 3130 recognize both the mouse and hclk-2 protein, and can thus be used in experiments with hclk-2 and in developing therapeutic/prophylactic agents for humans.

8.2.2 mclk-2 is a Phosphoprotein

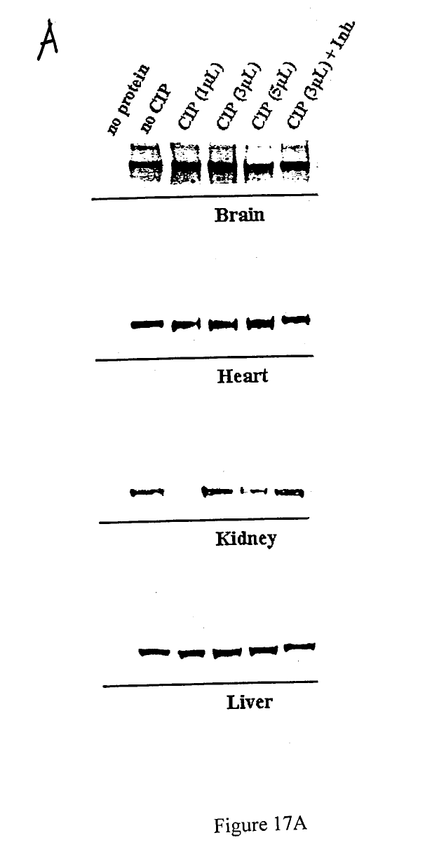

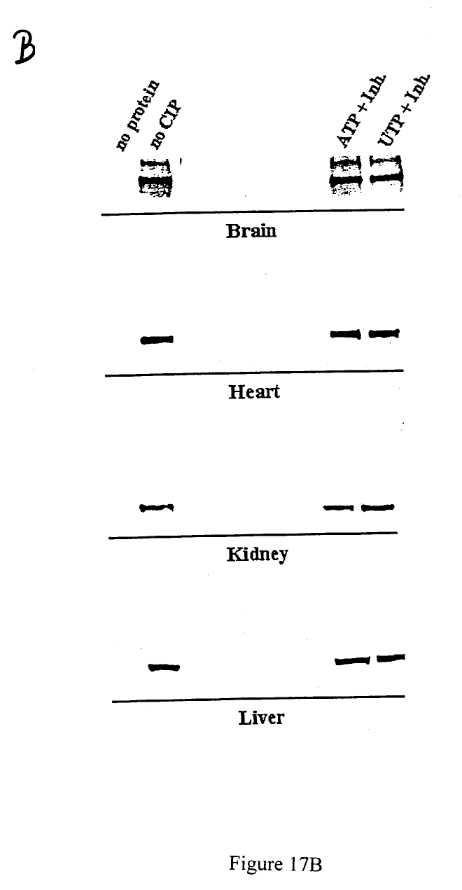

mclk-2 is post-translationally modified by phosphorylation. 50 μg of brain, heart, kidney or liver extracts was incubated at 37° C. for 30 min, with calf intestinal phosphatase (CIP), a non specific phosphatase, which dephosphorylates serine, threonine and tyrosine residues. After treatment with CIP, the band of the heart migrates lower (FIG. 17A). Control reactions include the non-treated extracts and extracts incubated with the phosphatase together with phosphatase inhibitors such as sodium fluoride, sodium glycerolphosphate and sodium orthovanadate at a final concentration of 5 mM.

A phosphorylation assay was conducted to see if kinase activity was present in the extracts that could phosphorylate mclk-2. Extracts were incubated with ATP. No shift in mclk-2 mobility was detected (FIG. 17B). Negative control reactions included UTP instead of ATP.

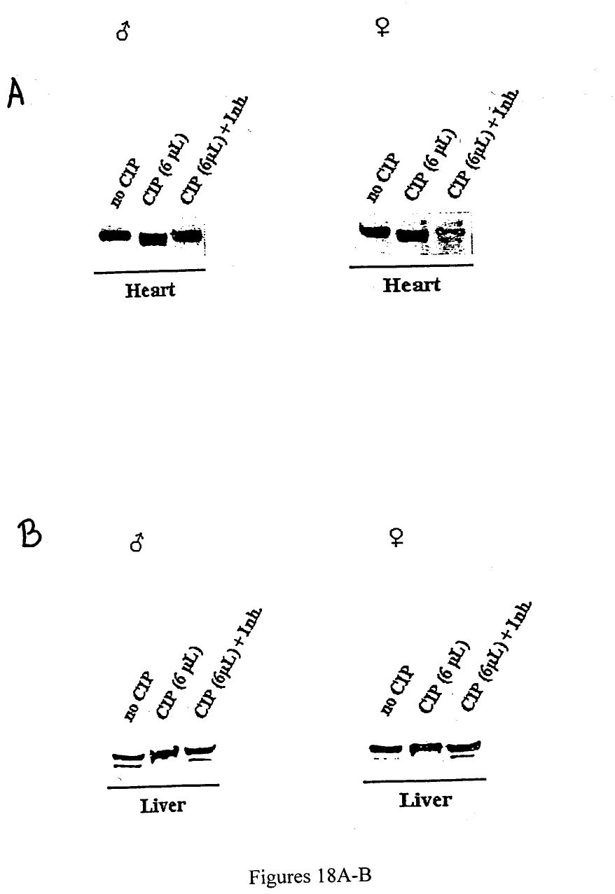

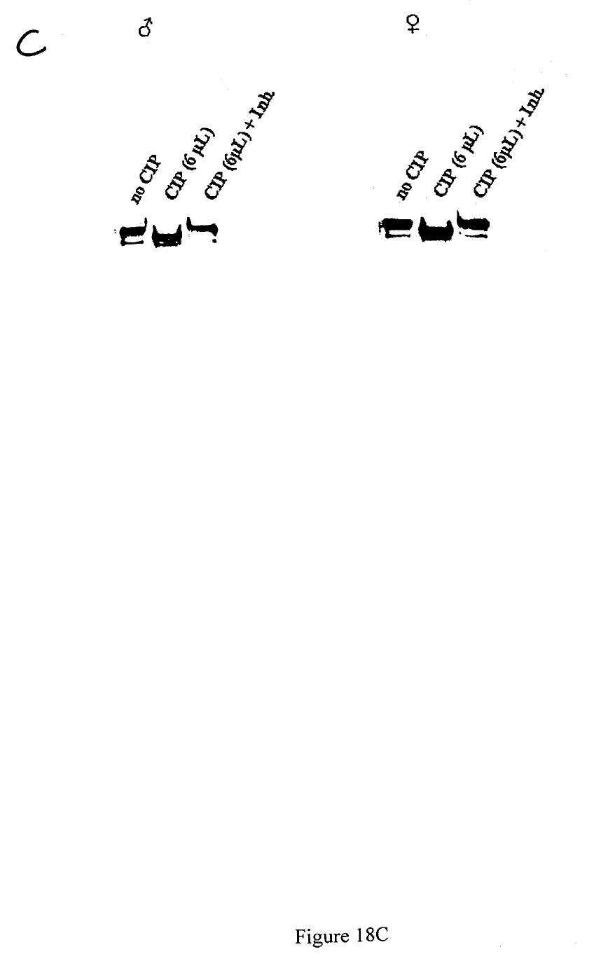

Further dephosphorylation experiments showed that mclk-2 can be more extensively dephosphorylated. Treatment with greater amounts of CIP and for a longer period of time (incubation for 30 min with 3 μl of CIP followed by another incubation of 30 min with an additional 3 μl of CIP), showed that mclk-2 in the heart and liver can be dephosphorylated further (FIGS. 18A-B). In addition, liver extracts were denatured for 5 min at 85° C. in 1% SDS and 100 mM DTT, prior to treatment with CIP. Both upper and the lower bands migrated significantly lower with this treatment (FIG. 18C). Given the magnitude of the effect observed on the denatured extracts, the denaturation of the extract prior to CIP treatment results in more phosphate groups. being exposed as substrates for the activity of the phosphatase. In conclusion, mclk-2 is a phosphoprotein which may be phosphorylated and dephosphorylated by kinases and phosphatases, respectively.

9. References Cited

All references cited herein are incorporated herein by reference in their entirety and for all purposes to the same extent as if each individual publication or patent or patent application was specifically and individually indicated to be incorporated by reference in its entirety for all purposes.

Many modifications and variations of this invention can be made without departing from its spirit and scope, as will be apparent to those skilled in the art. The specific embodiments described herein are offered by way of example only, and the invention is to be limited only by the terms of the appended claims, along with the full scope of equivalents to which such claims are entitled.

What is claimed is:

1. An isolated nucleic acid molecule selected from the group consisting of:
a) a nucleic acid molecule comprising a nucleotide sequence which is at least 90% identical to the nucleotide sequence of any of SEQ ID NOs: 1, 4, 5, 6, 7, 15, 16, 20, 21, 22, 23, 24, or a complement thereof;
b) a nucleic acid molecule that encodes a naturally occurring allelic variant of a polypeptide comprising the amino acid sequence of any of SEQ IDNOs:2, 3, 8, 9, 10, 11, 12, 13, 14, 17, 18, 19, 25, 26, 27, 28, 29, 30, 31, 32; and
c) a nucleic acid molecule that hybridizes with a nucleic acid probe consisting of the nucleotide sequence of any of SEQ ID NOs: 1, 4, 5, 6, 7, 15, 16, 20, 21, 22, 23, 24, or a complement thereof under the following conditions: hybridization in 6×sodium chloride/sodium citrate (SSC) at about 45° C. followed by one or more washes in 0.2×SSC, 0.1% SDS at 50-65° C.,
wherein said isolated nucleic acid does not comprise any of SEQ ID NOs: 1, 4, 5, 6, 7, 15, 16, 20, 21, 22, 23, 24.

2. The isolated nucleic acid molecule of claim 1 wherein said naturally occurring allelic variant occurs in humans.

3. The isolated nucleic acid molecule of claim 1 that is at least 90% identical to the nucleotide sequence of any of SEQ ID NOs: 1, 4, 5, 6, 7, 15, 16, 20, 21, 22, 23, 24 or a complement thereof, and hybridizes with a nucleic acid probe consisting of the nucleotide sequence of any of SEQ ID NOs: 1, 4, 5, 6, 7, 15, 16, 20, 21, 22, 23, 24, or a complement thereof under the following conditions: hybridization in 6×SSC at about 45° C. followed by one or more washes in 0.2×SSC, 0.1% SDS at 50-65° C.

4. The nucleic acid molecule of claim 1 further comprising nucleic acid equences encoding a heterologous polypeptide.

5. The nucleic acid molecule of claim 4 wherein the heterologous polypeptide is green fluorescent protein (GFP).

6. The nucleic acid molecule of claim 4 wherein the heterologous polypeptide targets localization to a cellular compartment.

7. The nucleic acid molecule of claim 6 wherein the cellular compartment is the mitochondria.

8. The nucleic acid molecule of claim 7 wherein the heterologous polypeptide is ornithine transcarbamylase.

9. A vector comprising a nucleic acid sequence of claim 1.

10. The vector of claim 9 that is an expression vector.

11. A host cell which comprises the vector of claim 9 or 10.

12. A host cell comprising a heterologous regulatory sequence that causes expression of a nucleic acid of claim 1.

13. The host cell of claim 11 or 12 which is a mammalian cell.

14. An isolated polypeptide selected from the group consisting of:
a) a naturally occurring allelic variant of a polypeptide comprising the amino acid sequence of any of SEQ ID NOs:2, 3, 8, 9, 10, 11, 12, 13, 14, 17, 18, 19, 25, 26, 27, 28, 29, 30, 31, 32, wherein the polypeptide is encoded by a nucleic acid molecule which hybridizes with a nucleic acid molecule consisting of the nucleotide sequence of any of SEQ ID NOs:1, 4, 5, 6, 7, 15, 16, 20, 21, 22, 23, 24, or a complement thereof under the following conditions: hybridization in 6×sodium chloride/sodium citrate (SSC) at about 45° C. followed by one or more washes in 0.2×SSC, 0.1% SDS at 50-65° C.;
b) a polypeptide that is encoded by a nucleic acid molecule comprising a nucleotide sequence which is at least 90% identical to a nucleic acid consisting of the nucleotide sequence of any of SEQ ID NOs: 1, 4, 5, 6, 7, 15, 16, 20, 21, 22, 23, 24 or a complement thereof; and
c) a polypeptide that is at least 90% identical to the amino acid sequence of any of SEQ ID NOs:2, 3, 8, 9, 10, 11, 12, 13, 14, 17, 18, 19, 25, 26, 27, 28, 29, 30, 31, 32;
wherein said isolated polypeptide does not comprise any of SEQ ID NOs:2, 3, 8, 9, 10, 11, 12, 13, 14, 17, 18, 19, 25, 26, 27, 28, 29, 30, 31, 32.

15. The isolated nucleic acid molecule of claim 14 wherein said naturally occurring allelic variant occurs in humans.

16. The polypeptide of claim 14 wherein the amino acid sequence of the polypeptide further comprises a heterologous amino acid sequence.

17. The polypeptide of claim 16 wherein the heterologous amino acid sequence encode green fluorescent protein (GFP).

18. A polyclonal antibody which immunospecifically binds the polypeptide of claim 14 but not a polypeptide consisting of any of SEQ ID NOs:2, 3, 8, 9, 10, 11, 12, 13, 14, 17, 18, 19, 25, 26, 27, 28, 29, 30, 31, 32.

19. A monoclonal antibody which immunospecifically binds the polypeptide of claim 14 but not a polypeptide consisting of any of SEQ ID NOs:2, 3, 8, 9, 10, 11, 12, 13, 14, 17, 18, 19, 25, 26, 27, 28, 29, 30, 31, 32.

20. The monoclonal antibody of claim 19 which is humanized.

21. A method for identifying a compound that specifically binds a clk-2 polypeptide comprising:
a) contacting the polypeptide with a compound under conditions and for a sufficient period of time that allows binding between the polypeptide and the compound; and
b) detecting binding of the compound to the polypeptide.

22. The method of claim 21 wherein said detecting comprises electrophoresis, immunoblotting, size exclusion chromatography, mass spectrometry, affinity chromatography, scintillation proximity assay, nuclear magnetic resonance spectroscopy, or fluorescence resonance energy transfer.

23. A method for identifying a compound which modulates the activity of a clk-2 polypeptide comprising:
a) contacting a cell expressing a clk-2 polypeptide with a compound under conditions and for a sufficient period of time for the compound to enter the cell; and
b) determining the activity of the clk-2 polypeptide in the cell;
wherein a difference in the activity of the clk-2 polypeptide as compared to the activity of the clk-2 polypeptide in the absence of the compound indicates that the compound modulates the activity of the clk-2 polypeptide.

24. The method of claim 23 wherein the level of clk-2 activity is determined by measuring telomere length in a cell, and wherein an increase in telomere length indicates an increase in the activity of the clk-2 polypeptide or a decrease in telomere length indicates a decrease in the activity of the clk-2 polypeptide.

25. The method of claim 23 wherein the level of clk-2 activity is determined by measuring the life span of the cell, and wherein an increase in the life span of the cell indicates a decrease in the activity of the clk-2 polypeptide or a decrease in the life span of the cell indicates an increase in the activity of the clk-2 polypeptide.

26. The method of claim 23 wherein the level of clk-2 activity is determined by measuring the rate of cell growth, and wherein an increase in the rate of cell growth indicates an increase in the activity of the clk-2 polypeptide or a decrease in rate of cell growth indicates a decrease in the activity of the clk-2 polypeptide.

27. The method of claim 23 further comprising treating the cell with hydroxyurea prior to determining the activity of the clk-2 polypeptide in the cell, wherein the level of clk-2 activity is determined by measuring apoptosis in the cell, and wherein an increase in apoptosis indicates an increase in the activity of the clk-2 polypeptide or a decrease in apoptosis indicates a decrease in the activity of the clk-2 polypeptide.

28. The method of claim 23 further comprising exposing the cell to oxidative stress prior to determining the activity of the clk-2 polypeptide in the cell, wherein the level of clk-2 activity is determined by measuring apoptosis in the cell, and wherein an increase in apoptosis indicates an increase in the activity of the clk-2 polypeptide or a decrease in apoptosis indicates a decrease in the activity of the clk-2 polypeptide.

29. The method of claim 28 wherein the cell is exposed to oxidative stress by treatment with menadione or t-butyl hydroperoxide.

30. A method for identifying a compound which modulates the activity of a clk-2 polypeptide comprising:
a) contacting a cell or organism with a compound, wherein the cell or organism exhibits at least one phenotype that is altered as a result of its expression of a mutant clk-2 polypeptide, when compared to a wild type cell or organism; and
b) determining the phenotype of said contacted cell or organism,
wherein a difference in the phenotype of said contacted cell or organism as compared to the phenotype of a cell or organism expressing the mutant clk-2 polypeptide not contacted with the compound indicates that the compound modulates the activity of a clk-2 polypeptide.

31. The method of claim 30 wherein the phenotype of the contacted cell or organism expressing a mutant clk-2 polypeptide is or approaches that of the phenotype of a cell or organism expressing a wild type clk-2 polypeptide.

32. The method of claim 30 wherein said cell expresses a loss-of-function mutant clk-2 polypeptide and said altered phenotype is selected from the group consisting of decreased telomere length, increased length of cell life, decreased cell growth rate, and decreased apoptosis in response to oxidative stress.

33. The method of claim 30 wherein said cell expresses a gain-of-function mutant clk-2 polypeptide and said altered phenotype is selected from the group consisting of increased telomere length, decreased length of cell life, increased cell growth rate, and increased apoptosis in response to oxidative stress.

34. The method of claim 30 wherein said organism is a Caenorhabditis elegans nematode.

35. The method of claim 30 wherein said organism is a Caenorhabditis elegans nematode and said mutant clk-2 polypeptide is a mouse clk-2 polypeptide or a variant thereof, or a human clk-2 polypeptide or variant thereof.

36. The method of claim 30 wherein said organism is a Caenorhabditis elegans nematode, said mutant clk-2 polypeptide is a loss-of-function mutant and said altered phenotype is selected from the group consisting of increased telomere length, increased length of life, decreased cell growth rate, slower embryonic development, slower post-embryonic development, slower defecation cycles, lower pharyngeal pumping rate, smaller self-brood size, and lower peak egg-laying rate.

37. The method of claim 30 wherein said organism is a Caenorhabditis elegans nematode, and said mutant clk-2 polypeptide is encoded by clk-2(qm37).

38. The method of claim 30 wherein said organism is a Caenorhabditis elegans nematode, said mutant clk-2 polypeptide is a gain-of-function mutant and said altered phenotype is selected from the group consisting of decreased telomere length, decreased length of life, increased cell growth rate, faster embryonic development, faster post-embryonic development, faster defecation cycles, higher pharyngeal pumping rate, larger self-brood size, and higher peak egg-laying rate.

39. A method of identifying a compound that modulates clk-2 expression comprising:
a) contacting a recombinant cell with a compound, said recombinant cell comprising a reporter gene operably associated with a regulatory sequence of a clk-2 gene, such that expression of the reporter gene is regulated by the regulatory sequence; and
b) determining the level of expression of said reporter gene in said contacted recombinant cell,
wherein a difference in the expression level of said reporter gene in said contacted recombinant cell as compared to the expression level of said reporter gene in said recombinant cell not contacted with the compound indicates that the compound modulates clk-2 expression.

40. The method of claim 39 wherein said recombinant cell is a mammalian cell.

41. The method of claim 39 wherein a recombinant Caenorhabditis elegans nematode comprises said recombinant cell.

42. A method of identifying a compound that modulates the expression of a clk-2 nucleic acid or polypeptide comprising:
a) contacting a cell with a compound, and
b) determining the level of expression of the clk-2 nucleic acid or polypeptide in said contacted recombinant cell,
wherein a difference in the expression level of the clk-2 nucleic acid or polypeptide in said contacted recombinant cell as compared to the expression level of the clk-2 nucleic acid or polypeptide in said recombinant cell not contacted with the compound indicates that the compound modulates clk-2 expression.

43. The method of claim 42 wherein said cell is a mammalian cell.

44. The method of claim 42 wherein a Caenorhabditis elegans nematode comprises said cell.

45. A method for identifying an agent that modulates the phosphorylation level of a clk-2 polypeptide comprising:
a) contacting a reaction mixture with a compound, said mixture comprising clk-2 and at least one polypeptide capable of phosphorylating or dephosphorylating clk-2; and
b) determining the phosphorylation level of clk-2 in said mixture, wherein a difference in the phosphorylation level of clk-2 as compared to the phosphorylation level of clk-2 in a mixture not contacted with said compound indicates that the compound modulates the phosphorylation level of a clk-2 polypeptide.

46. A transgenic non-human animal comprising cells that contain a transgenic regulatory sequence such that a progeny of said transgenic non-human animal inherits said transgene wherein said regulatory sequence controls the expression of a clk-2 protein.

47. The transgenic animal of claim 46 wherein said clk-2 protein is expressed from a transgenic clk-2 nucleic acid.

48. A transgenic non-human animal comprising cells that contain a transgenic nucleic acid encoding a polypeptide of claim 14 such that a progeny of said transgenic non-human animal inherits said transgenic nucleic acid.

49. The transgenic animal of claim 46 or 48 wherein said animal is a Caenorhabditis elegans nematode.

50. The transgenic animal of claim 46 or 48 wherein said animal is a mouse.

51. The transgenic animal of claim 47 or 48 wherein said transgenic nucleic acid is from a species other than that of said transgenic animal.

52. The transgenic animal of claim 51 wherein said transgenic nucleic acid is human.

53. A non-human transgenic animal, wherein the animal carries a disruption in an endogenous clk-2 gene such that said animal exhibits an altered phenotype relative to a wild type animal.

54. The transgenic animal of claim 53 wherein said altered phenotype is an increased life span.

55. The transgenic animal of claim 53 wherein said animal is a Caenorhabditis elegans nematode and said altered phenotype is an increased telomere length.

56. The transgenic animal of claim 53 wherein said animal is a mouse and said altered phenotype is a decreased telomere length.

57. A method of treating or preventing a disorder associated with excess clk-2 polypeptide activity in a subject comprising administering to a subject in which such treatment or prevention is desired an effective amount of a compound that decreases clk-2 polypeptide activity or clk-2 gene expression.

58. The method of claim 57 wherein the disorder is characterized by the presence of cells exhibiting increased telomere length and/or increased apoptosis.

59. The method of claim 58 wherein the disorder associated with increased telomere length is cancer.

60. The method of claim 59 wherein said apoptosis is caused by oxidative stress.

61. The method of claim 58 wherein said disorder associated with increased apoptosis is a neurodegenerative disorder.

62. The method of claim 61 wherein said neurodegenerative disorder is Parkinson's Disease or Alzheimer's Disease, Huntington's Chorea, or amyotrophic lateral sclerosis.

63. A method of treating or preventing a disorder associated with deficient clk-2 polypeptide activity in a subject comprising administering to a subject in which such treatment or prevention is desired an effective amount of a compound that increases clk-2 polypeptide activity or clk-2 gene expression.

64. The method of claim 63, wherein the disorder is characterized by the presence of cells exhibiting decreased telomere length and/or decreased apoptosis.

65. The method of claim 64 wherein the disorder associated with decreased telomere length is accelerated aging.

66. The method of claim 64 wherein the disorder associated with decreased apoptosis is cancer.

67. The method of claim 66 wherein said cancer is colorectal cancer, breast cancer, or skin cancer.

68. The method of claim 64 wherein the disorder associated with decreased apoptosis is an autoimmune disorder.

69. The method of claim 66 or 68 wherein said compound that increases clk-2 polypeptide activity or clk-2 gene expression is administered in combination with an apoptosis-causing therapeutically effective agent.

70. The method of any of claims 57 or 63 wherein said compound is conjugated to an antibody that immunospecifically binds a cell associated with the disorder.

71. A method for extending the life of a cell comprising (i) increasing expression of a clk-2 nucleic acid or polypeptide, (ii) introducing into the cell and expressing a clk-2 nucleic acid, (iii) introducing into the cell a clk-2 polypeptide, or (iv) contacting the cell with a compound that increases clk-2 expression or activity.

72. A method for extending the life of a multicellular animal or plant comprising (i) increasing expression of a clk-2 nucleic acid or polypeptide, (ii) introducing into the animal or plant and expressing a clk-2 nucleic acid, (iii) introducing into the animal or plant a clk-2 polypeptide, or (iv) contacting the animal or plant with a compound that increases clk-2 expression or activity.

73. A method of accelerating the growth of a multicellular animal or plant comprising (i) increasing expression of a clk-2 nucleic acid or polypeptide (ii) introducing into the animal or plant and expressing a clk-2 nucleic acid, (iii) introducing into the animal or plant a clk-2 polypeptide, or (iv) contacting the animal or plant with a compound that increases clk-2 expression or activity.

74. A method of decreasing the growth of a tissue or organ comprising (i) decreasing expression of a clk-2 nucleic acid or polypeptide (ii) introducing into the tissue or organ and expressing a clk-2 double stranded interfering RNA, (iii) introducing into the tissue or organ a compound that interferes with the activity of the clk-2 polypeptide, or (iv) contacting the tissue or organ with a compound that decreases clk-2 expression or activity.
